SEQUENCE LISTING (46 sequences)

SEQ ID NO: 1 - protein, 119 aa, Artificial Sequence [sequence omitted]

SEQ ID NO: 2 - protein, 111 aa, Artificial Sequence [sequence omitted]

SEQ ID NO: 3 - protein, 5 aa, Artificial Sequence
  source: /note=“Description of Artificial Sequence Synthetic peptide”
HYVMA

SEQ ID NO: 4 - protein, 17 aa, Artificial Sequence
  source: /note=“Description of Artificial Sequence Synthetic peptide”
SISSSGGWTLYADSVKG

SEQ ID NO: 5 - protein, 10 aa, Artificial Sequence
  source: /note=“Description of Artificial Sequence Synthetic peptide”
GLKMATIFDY

SEQ ID NO: 6 - protein, 14 aa, Artificial Sequence
  source: /note=“Description of Artificial Sequence Synthetic peptide”
TGTSSDVGSYNVVS

SEQ ID NO: 7 - protein, 7 aa, Artificial Sequence
  source: /note=“Description of Artificial Sequence Synthetic peptide”
EVSQRPS

SEQ ID NO: 8 - protein, 11 aa, Artificial Sequence
  source: /note=“Description of Artificial Sequence Synthetic peptide”
CSYAGSSIFVI

SEQ ID NO: 9 - protein, 118 aa, Artificial Sequence
  source: /note=“Description of Artificial Sequence Synthetic polypeptide”
EVQLLESGGGLVQPGGSLRLSCAASGFTFSAYNMRWVRQAPGKGLEWVSVIYPSGGATRYADSVKGRFTISRDNSKNTLYLQMNSLRAEDTAVYYCARGYYYYGMDVWGQGTLVTVSS

SEQ ID NO: 10 - protein, 110 aa, Artificial Sequence
  source: /note=“Description of Artificial Sequence Synthetic polypeptide”
QSVLTQPPSASGTPGQRVTISCSGSDSNIGRNYIYWYQQFPGTAPKLLIYRNNQRPSGVPDRISGSKSGTSASLAISGLRSEDEAEYHCGTWDDSLSGPVFGGGTKLTVL

SEQ ID NO: 11 - protein, 5 aa, Artificial Sequence
  source: /note=“Description of Artificial Sequence Synthetic peptide”
AYNMR

SEQ ID NO: 12 - protein, 17 aa, Artificial Sequence
  source: /note=“Description of Artificial Sequence Synthetic peptide”
VIYPSGGATRYADSVKG

SEQ ID NO: 13 - protein, 9 aa, Artificial Sequence
  source: /note=“Description of Artificial Sequence Synthetic peptide”
GYYYYGMDV

SEQ ID NO: 14 - protein, 13 aa, Artificial Sequence
  source: /note=“Description of Artificial Sequence Synthetic peptide”
SGSDSNIGRNYIY

SEQ ID NO: 15 - protein, 7 aa, Artificial Sequence
  source: /note=“Description of Artificial Sequence Synthetic peptide”
RNNQRPS

SEQ ID NO: 16 - protein, 11 aa, Artificial Sequence
  source: /note=“Description of Artificial Sequence Synthetic peptide”
GTWDDSLSGPV

SEQ ID NO: 17 - protein, 122 aa, Artificial Sequence
  source: /note=“Description of Artificial Sequence Synthetic polypeptide”
EVQLLESGGGLVQPGGSLRLSCAASGFTFSAYGMGWVRQAPGKGLEWVSYISPSGGHTKYADSVKGRFTISRDNSKNTLYLQMNSLRAEDTAVYYCAKVLETGLLVDAFDIWGQGTMVTVSS

SEQ ID NO: 18 - protein, 106 aa, Artificial Sequence
  source: /note=“Description of Artificial Sequence Synthetic polypeptide”
QYELTQPPSVSVYPGQTASITCSGDQLGSKFVSWYQQRPGQSPVLVMYKDKRRPSEIPERFSGSNSGNTATLTISGTQAIDEADYYCQAWDSSTYVFGTGTKVTVL

SEQ ID NO: 19 - protein, 5 aa, Artificial Sequence
  source: /note=“Description of Artificial Sequence Synthetic peptide”
AYGMG

SEQ ID NO: 20 - protein, 17 aa, Artificial Sequence
  source: /note=“Description of Artificial Sequence Synthetic peptide”
YISPSGGHTKYADSVKG

SEQ ID NO: 21 - protein, 13 aa, Artificial Sequence
  source: /note=“Description of Artificial Sequence Synthetic peptide”
VLETGLLVDAFDI

SEQ ID NO: 22 - protein, 11 aa, Artificial Sequence
  source: /note=“Description of Artificial Sequence Synthetic peptide”
SGDQLGSKFVS

SEQ ID NO: 23 - protein, 8 aa, Artificial Sequence
  source: /note=“Description of Artificial Sequence Synthetic peptide”
YKDKRRPS

SEQ ID NO: 24 - protein, 9 aa, Artificial Sequence
  source: /note=“Description of Artificial Sequence Synthetic peptide”
QAWDSSTYV

SEQ ID NO: 25 - protein, 118 aa, Artificial Sequence
  source: /note=“Description of Artificial Sequence Synthetic polypeptide”
EVQLLESGGGLVQPGGSLRLSCAASGFTFSWYGMGWVRQAPGKGLEWVSYISPSGGITVYADSVKGRFTISRDNSKNTLYLQMNSLRAEDTAVYYCARLNYYYGLDVWGQGTTVTVSS

SEQ ID NO: 26 - protein, 108 aa, Artificial Sequence
  source: /note=“Description of Artificial Sequence Synthetic polypeptide”
QDIQMTQSPSSLSASVGDRITITCQASQDIGDSLNWYQQKPGKAPRLLIYDASNLETGVPPRFSGSGSGTDFTFTFRSLQPEDIATYFCQQSANAPFTFGPGTKVDIK

SEQ ID NO: 27 - protein, 5 aa, Artificial Sequence
  source: /note=“Description of Artificial Sequence Synthetic peptide”
WYGMG

SEQ ID NO: 28 - protein, 17 aa, Artificial Sequence
  source: /note=“Description of Artificial Sequence Synthetic peptide”
YISPSGGITVYADSVKG

SEQ ID NO: 29 - protein, 9 aa, Artificial Sequence
  source: /note=“Description of Artificial Sequence Synthetic peptide”
LNYYYGLDV

SEQ ID NO: 30 - protein, 11 aa, Artificial Sequence
  source: /note=“Description of Artificial Sequence Synthetic peptide”
QASQDIGDSLN

SEQ ID NO: 31 - protein, 7 aa, Artificial Sequence
  source: /note=“Description of Artificial Sequence Synthetic peptide”
DASNLET

SEQ ID NO: 32 - protein, 9 aa, Artificial Sequence
  source: /note=“Description of Artificial Sequence Synthetic peptide”
QQSANAPFT

SEQ ID NO: 33 - protein, 119 aa, Artificial Sequence
  source: /note=“Description of Artificial Sequence Synthetic polypeptide”
EVQLLESGGGLVQPGGSLRLSCAASGFTFSRYGMWWVRQAPGKGLEWVSYIGSSGGPTYYVDSVKGRFTISRDNSKNTLYLQMNSLRAEDTAVYYCAGGRGTPYYFDSWGQGTLVTVSS

SEQ ID NO: 34 - protein, 110 aa, Artificial Sequence
  source: /note=“Description of Artificial Sequence Synthetic polypeptide”
QYELTQPASVSGSPGQSITISCTGTSSDIGRWNIVSWYQQHPGKAPKLMIYDVSNRPSGVSNRFSGSKSGNTASLTISGLQAEDEADYYCSSYTSSSTWVFGGGTKLTVL

SEQ ID NO: 35 - protein, 5 aa, Artificial Sequence
  source: /note=“Description of Artificial Sequence Synthetic peptide”
RYGMW

SEQ ID NO: 36 - protein, 17 aa, Artificial Sequence
  source: /note=“Description of Artificial Sequence Synthetic peptide”
YIGSSGGPTYYVDSVKG

SEQ ID NO: 37 - protein, 10 aa, Artificial Sequence
  source: /note=“Description of Artificial Sequence Synthetic peptide”
GRGTPYYFDS

SEQ ID NO: 38 - protein, 14 aa, Artificial Sequence
  source: /note=“Description of Artificial Sequence Synthetic peptide”
TGTSSDIGRWNIVS

SEQ ID NO: 39 - protein, 7 aa, Artificial Sequence
  source: /note=“Description of Artificial Sequence Synthetic peptide”
DVSNRPS

SEQ ID NO: 40 - protein, 10 aa, Artificial Sequence
  source: /note=“Description of Artificial Sequence Synthetic peptide”
SSYTSSSTWV

SEQ ID NO: 41 - protein, 1323 aa, Artificial Sequence
  source: /note=“Description of Artificial Sequence Synthetic polypeptide”
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 42 - protein, 445 aa, Artificial Sequence
  source: /note=“Description of Artificial Sequence Synthetic polypeptide”
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 43 - protein, 217 aa, Artificial Sequence
  source: /note=“Description of Artificial Sequence Synthetic polypeptide”
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 44 - protein, 1095 aa, Homo sapiens
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSASTGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGAASDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLAAALQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEWLGVWGQGTLVTVSSGGGGSSGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLG

SEQ ID NO: 45 - DNA, 357 bp, Artificial Sequence
  source: /note=“Description of Artificial Sequence Synthetic polynucleotide”
gaggtgcagctgctggagagcggcggagggctggtccagccaggcggcagcctgaggctgtcctgcgccgccagcggcttcaccttcagccactacgtgatggcctgggtgcggcaggccccaggcaagggcctggaatgggtgtccagcatcagcagcagcggcggctggaccctgtacgccgacagcgtgaagggcaggttcaccatcagcagggacaacagcaagaacaccctgtacctgcagatgaacagcctgagggccgaggacaccgccgtgtactactgcaccaggggcctgaagatggccaccatcttcgactactggggccagggcaccctggtgaccgtgagcagc

SEQ ID NO: 46 - DNA, 333 bp, Artificial Sequence
  source: /note=“Description of Artificial Sequence Synthetic polynucleotide”
cagtccgccctgacccagcccgccagcgtgagcggcagcccaggccagagcatcaccatcagctgcaccggcaccagcagcgacgtgggcagctacaacgtggtgtcctggtatcagcagcaccccggcaaggcccccaagctgatcatctacgaggtgtcccagaggcccagcggcgtgagcaacaggttcagcggcagcaagagcggcaacaccgccagcctgaccatcagcggcctgcagaccgaggacgaggccgactactactgctgcagctacgccggcagcagcatcttcgtgatcttcggcggagggaccaaggtgaccgtccta

(19) United States
(12) Patent Application Publication
(10) Pub. No.: US 2015/0231238 A1
(43) Pub. Date: Aug. 20, 2015

(54) OVERCOMING RESISTANCE TO ERBB PATHWAY INHIBITORS

(71) Applicants: Gabriela Garcia, Roslindale, MA (US); William Kubasek, Belmont, MA (US); Maria Johanna Lahdenranta, Cambridge, MA (US); Gavin MacBeath, Wakefield, MA (US); Charlotte McDonagh, Winchester, MA (US); Victor Moyo, Rinyoes, NJ (US); Matthew David Onsum, San Francisco, CA (US); Birgit Schoeberl, Cambridge, MA (US); Mark Sevecka, Cambridge, MA (US); Marisa Wainszelbaum, Boston, MA (US); Bo Zhang, Lynnfield, MA (US)

(72) Inventors: Gabriela Garcia, Roslindale, MA (US); William Kubasek, Belmont, MA (US); Maria Johanna Lahdenranta, Cambridge, MA (US); Gavin MacBeath, Wakefield, MA (US); Charlotte McDonagh, Winchester, MA (US); Victor Moyo, Rinyoes, NJ (US); Matthew David Onsum, San Francisco, CA (US); Birgit Schoeberl, Cambridge, MA (US); Mark Sevecka, Cambridge, MA (US); Marisa Wainszelbaum, Boston, MA (US); Bo Zhang, Lynnfield, MA (US)

(73) Assignee: MERRIMACK PHARMACEUTICALS, INC., Cambridge, MA (US)

(21) Appl. No.: 14/004,848

(22) PCT Filed: Mar. 15, 2012
(86) PCT No.: PCT/US2012/029292

§ 371 Date: Apr. 16, 2015

Related U.S. Application Data
(60) Provisional application No. 61/452,976, filed on Mar. 15, 2011.
(60) Provisional application No. 61/452,974, filed on Mar. 15, 2011.

(51) Int. Cl. A61K 39/395; A61K 45/06; A61K 31/138; A61K 31/337

(52) U.S. Cl. CPC A61K 39/39558; A61K 31/337; A61K 45/06; A61K 31/138; A61K 2039/507

(57) ABSTRACT

Provided are methods for overcoming resistance to an ErbB pathway inhibitor, such as an EGFR inhibitor or a HER2 inhibitor. The resistance may be acquired resistance to an EGFR inhibitor, such as acquired resistance to gefitinib. In the methods provided, a subject exhibiting resistance to an ErbB pathway inhibitor is selected and both an ErbB 3 inhibitor and a second ErbB pathway inhibitor are administered to the subject, such as an EGFR inhibitor or a HER2 inhibitor. Also provided are methods for inhibiting the growth of a tumor comprising a T790M EGFR mutation by contacting the tumor with an ErbB3 inhibitor and an EGFR inhibitor. Compositions for overcoming resistance to an ErbB pathway inhibitor, comprising both an ErbB 3 inhibitor and a second ErbB pathway inhibitor, such as an EGFR inhibitor or a HER2 inhibitor, are also provided.

### BACKGROUND

The ERBB family of receptor tyrosine kinases include EGFR (HER1), HER2 (ErbB2), ERBB3 (HER3), and ERBB4 (HER4). Over the past ten years, it has become evident that many epithelial cancers require EGFR or HER2 signaling for their growth and survival. Agents targeting EGFR have become widely used for the treatment of cancer such as lung cancer, colorectal cancer, head and neck cancer and, typically in combination with gemcitabine, pancreatic cancer. For example, small molecule tyrosine kinase inhibitors (TKIs) that downregulate the EGFR signaling pathway have been developed, such as gefitinib (Iressa®), which is indicated for the treatment of non-small cell lung cancer, erlotinib (Tarceva®), which is indicated for the treatment of non-small cell lung cancer and pancreatic cancer, and lapatinib (Tykerb®), which is indicated for the treatment of HER2-positive breast cancer. Furthermore, antibodies specific for EGFR have been developed, such as the humanized monoclonal antibody cetuximab (Erbitux®), which is indicated for the treatment of colorectal cancer and head and neck cancer. Agents targeting HER2 also have become widely used for the treatment of cancer such as breast cancer. An example of an agent targeting HER2 is the humanized anti-HER2 monoclonal antibody trastuzumab (Herceptin®), which is used around the world for the treatment of HER2 overexpressing breast cancer.

Recent studies have found that cancers that are sensitive to EGFR inhibitors are unique in that phosphoinositide 3-kinase (PI3K) signaling is under the sole control of EGFR. For the EGFR inhibitors to be effective, they must cause downregulation of the PI3K/AKT pathway (Bianco, R. et al. (2003) Oncogene 22:2812-2822). While patients with EGFR-driven cancers often initially respond well to EGFR-targeted therapies, over time many patients that were initially responsive suffer from recurrence and develop tumors refractory to the original treatment. Furthermore, certain EGFR-positive cancers exhibit a predisposition to resistance to EGFR-targeted therapies. One way in which such resistance has been observed to develop is through mutation of EGFR. The “T790M EGFR mutation” comprises and is identified by a change of a threonine, which is present at position 790 of wild-type EGFR, to a methionine. This mutation, which locates to the kinase domain of EGFR, has been described, e.g., by Kobayashi, S. et al. (2005) N. Engl. J. Med. 352:786-792; and Pao, W. et al. (2005) PLoS Med. 2:225-235.

Persistent activation of the PI3K signaling pathway through ErbB3 also has been associated with gefitinib resistance (Engelman et al. (2005), supra; Engelman et al. (2007) supra).

Resistance to HER2 inhibitors, such as trastuzumab and lapatinib, also has been reported. Similar to EGFR inhibitor resistance, persistent activation of the PI3K/AKT signaling pathway is at least one of the mechanisms reported for the acquired resistance to HER2 inhibitors.

Studies have identified ErbB3, which is a kinase-dead member of the ErbB family, as an activator of PI3K/AKT signaling in EGFR dependent cancers (Engelman, J. A. et al. (2005) Proc. Natl. Acad. Sci. USA 102:3788-3793; Engelman, J. A. et al. (2007) Science 316:1039-1045; In these cells, ErbB3 is tyrosine phosphorylated in an EGFR-dependent manner and then directly binds PI3K. Upon inhibition of EGFR, ErbB3 phosphorylation is lost, it no longer binds PI3K and there is loss of PI3K/AKT signaling (Engelman, J. A. et al. (2005) Proc. Natl. Acad. Sci. USA 102:3788-3793; Engelman, J. A. et al. (2007) Science 316:1039-1045). Furthermore, downregulation of ErbB3 using short hairpin RNA (shRNA) leads to a decrease in AKT phosphorylation in EGFR dependent cancers (Engelman, J. A. et al. (2005) Proc. Natl. Acad. Sci. USA 102:3788-3793). Similarly, ErbB3 is a major activator of PI3K in HER2 amplified breast cancers. Trastuzumab treatment leads to loss of ErbB3 phosphorylation, dissociation between ErbB3 and PI3K and loss of AKT phosphorylation in these cancers. Thus, signaling through ErbB3 is thought to be a major mechanism of PI3K/AKT activation in both EGFR and HER2 driven cancers.

There are also examples of resistance that implicate EGFR, HER2 and MET in reactivating ErbB3 (Engelman, J. A. et al. (2006) J. Clin. Invest. 116:2695-2706; Engelman, J. A. et al. (2007) Science 316:1039-1045; Ritter, C. A. et al. (2007) Clin. Cancer Res. 13:4909-4919; Sergina, N. V. et al. (2007) Nature 445:437-441). In addition, heregulin-induced activation of HER2-ErbB3 heterodimers has also been associated with resistance to EGFR inhibitors (Zhou, B. B. et al. (2006) Cancer Cell 10:39-50).

In view of the foregoing, compositions and methods for overcoming resistance to ErbB pathway inhibitors, including EGFR inhibitors (such as TKIs and anti-EGFR antibodies) and HER2 inhibitors (such as TKIs and anti-HER2 antibodies), are being actively sought, as they promise to extend or restore the effectiveness of targeted cancer therapies.

### SUMMARY

Methods are provided for overcoming resistance to ErbB pathway inhibitors, as well as pharmaceutical compositions that can be used in the practice of such methods. The methods and compositions provided herein are based, at least in part, on the discovery by the inventors that use of an ErbB3 inhibitor in combination with an ErbB pathway inhibitor can overcome tumor resistance to an ErbB pathway inhibitor. For example, use of a bispecific anti-ErbB3, anti-ErbB2 antibody (or an anti-ErbB3 antibody) in combination with an anti-EGFR antibody has been demonstrated to overcome acquired resistance in vivo to the small molecule EGFR inhibitor gefitinib.

Accordingly, in one aspect, a method for overcoming or preventing resistance of a tumor to an ErbB pathway inhibitor in a subject is provided, the method comprising:

selecting a subject with a tumor exhibiting, or at risk for developing, resistance to an ErbB pathway inhibitor; and

administering to the subject (i) an ErbB3 inhibitor and (ii) an ErbB pathway inhibitor.

The ErbB pathway inhibitor administered to the subject does not need to be the same ErbB pathway inhibitor to which the subject is resistant, although typically, the ErbB pathway inhibitor administered to the subject will be directed against the same ErbB pathway as the ErbB pathway inhibitor to which the subject is resistant. For example, a subject who exhibits resistance to the EGFR pathway inhibitor gefitinib may be co-administered an ErbB3 inhibitor and an EGFR inhibitor, which EGFR inhibitor can be, for example, gefitinib or the anti-EGFR antibody cetuximab.

In certain embodiments, the resistance exhibited by the subject to the ErbB pathway inhibitor is acquired resistance. In one embodiment, the acquired resistance is acquired resistance to an EGFR inhibitor, such as acquired resistance wherein the EGFR in the tumor comprises tumor cells comprising an EGFR gene comprising a T790M EGFR mutation. In another embodiment the tumor comprises tumor cells comprising a KRAS gene comprising at least one KRAS mutation, e.g., a G12S, G12C, or G12V KRAS mutation or a Q61R KRAS mutation. In one embodiment, the acquired resistance is resistance to gefitinib. In another embodiment, the acquired resistance is acquired resistance to a HER2 inhibitor, such as acquired resistance to trastuzumab. In various embodiments, the resistance exhibited by the subject is associated with reactivation of PI3K/AKT signaling in tumor cells in the subject.

In one embodiment, the ErbB3 inhibitor administered to the subject is an anti-ErbB3 antibody. An exemplary anti-ErbB3 antibody is MM-121 (Ab #6), comprising V_{H} and V_{L} sequences as shown in SEQ ID NOs: 1 and 2, respectively. Alternately, the anti-ErbB3 antibody is an antibody that comprises the heavy and light chain CDRs of MM-121 (i.e., the anti-ErbB3 antibody comprises an antibody comprising V_{H} CDR1, 2 and 3 sequences as shown in SEQ ID NOs: 3-5, respectively, and V_{L} CDR1, 2 and 3 sequences as shown in SEQ ID NOs: 6-8, respectively). In another embodiment, the anti-ErbB3 antibody is an antibody having heavy and light chains comprising the amino acid sequences set forth in SEQ ID NOs 42 and 43, respectively. In other embodiments, the anti-ErbB3 antibody is Ab #3 (comprising V_{H} and V_{L} sequences as shown in SEQ ID NOs: 9 and 10, respectively), Ab #14 (comprising V_{H} and V_{L} sequences as shown in SEQ ID NOs: 17 and 18, respectively), Ab #17 (comprising V_{H} and V_{L} sequences as shown in SEQ ID NOs: 25 and 26, respectively) or Ab #19 (comprising V_{H} and V_{L} sequences as shown in SEQ ID NOs: 33 and 34, respectively). In still other embodiments, the anti-ErbB3 antibody is selected from mAb 1B4C3 or mAb 2D1D12 or humanized versions thereof, mAb 205.10 (e.g., mAb 205.10.1, mAb 205.10.2, or mAb 205.10.3) (Roche-Glycart), AMG-888 (U3-1287—U3 Pharma AG and Amgen), AV-203 (Aveo Pharmaceuticals) and humanized mAb 8B8. Typically, the ErbB3 inhibitor inhibits PI3K/AKT signaling.

In one embodiment, the ErbB3 inhibitor administered to the subject is a bispecific anti-ErbB3, anti-ErbB2 antibody such as MM-111, which comprises two scFvs in a Human Serum Albumin (HSA) conjugate as set forth in SEQ ID NO:44. MM-111 abrogates heregulin binding to ErbB2/ErbB3 and inhibits heregulin activation of ErbB2/ErbB3 without significantly affecting ErbB2 biological activity. A number of bispecific anti-ErbB2/antiErbB3 antibodies that are scFv HSA conjugates, including MM-111 (also referred to as B2B3-1), as well as A5-HSA-ML3.9, A5-HSA-B1D2, B12-HSA-B1D2, A5-HSA-F5B6H2, H3-HSA-F5B6H2, and F4-HSA-F5B6H2, are described in co-pending U.S. patent application Publication No. 20110059076, and PCT publication number WO2009/126920. Other suitable bispecific anti-ErbB2/antiErbB3 antibodies are disclosed and claimed in U.S. Pat. Nos. 7,332,580 and 7,332,585. MM-111 is currently undergoing clinical trials, including an open-label Phase 1-2 and pharmacologic study of MM-111 in patients with advanced, refractory HER2 positive cancers, and an open-label Phase 1-2 trial of MM-111 in combination with trastuzumab (Herceptin®) in patients with advanced HER2 positive breast cancer. In certain embodiments, the ErbB3 inhibitor inhibits PI3K/AKT signaling.

In one embodiment, the ErbB pathway inhibitor administered to the subject is an EGFR inhibitor. For example, the EGFR inhibitor can be an anti-EGFR antibody such as cetuximab. Other exemplary anti-EGFR antibodies are MM-151, Sym004, matuzumab, panitumumab, nimotuzumab and mAb 806.

In another embodiment, the EGFR inhibitor administered to the subject is a small molecule inhibitor of EGFR signaling such as gefitinib. Other exemplary small molecule inhibitors of EGFR signaling are afatinib, lapatinib, canertinib, erlotinib HCL, pelitinib, PKI-166, PD158780, and AG 1478.

In another embodiment, the ErbB pathway inhibitor administered to the subject is a HER2 inhibitor. For example, the HER2 inhibitor can be an anti-HER2 antibody such as trastuzumab. Alternatively, the HER2 inhibitor administered to the subject can be a small molecule inhibitor of HER2 signaling such as lapatinib.

In another aspect, a method of inhibiting growth of a tumor comprising cells comprising a T790M EGFR mutation cells is disclosed, the method comprising contacting the tumor with (i) an EGFR inhibitor; and (ii) an ErbB3 inhibitor. In a one embodiment, the ErbB3 inhibitor comprises ananti-ErbB3 antibody, such as one or more of the anti-ErbB3 antibodies set forth above. In another embodiment, the ErbB3 inhibitor comprises a bispecific anti-ErbB3, anti-ErbB2 antibody, such as one or more of the bispecific antibodies set forth above. In one embodiment, the ErbB3 inhibitor inhibits PI3K/AKT signaling. In another embodiment, the EGFR inhibitor comprises an anti-EGFR antibody, such as one or more of the antibodies set forth above. In yet another embodiment, the EGFR inhibitor comprises a small molecule inhibitor of EGFR signaling, such as one or more of the small molecule inhibitors set forth above.

In yet another aspect, a method of treating a tumor in a subject is provided, the method comprising

selecting a subject with a tumor that, by biopsy tests positive for a T790M EGFR mutation, and

administering to the subject (i) an EGFR inhibitor; and (ii) an ErbB3 inhibitor. In certain embodiments, the ErbB3 inhibitor administered to the subject comprises an anti-ErbB3 antibody, such as one or more of the anti-ErbB3 antibodies set forth above. In certain embodiments, the ErbB3 inhibitor administered to the subject comprises a bispecific anti-ErbB3, anti-ErbB2 antibody, such as one or more of the bispecific antibodies set forth above. Exemplary ErbB3 inhibitors inhibit PI3K/AKT signaling. In another embodiment, the EGFR inhibitor administered to the subject comprises an anti-EGFR antibody, such as one or more of the antibodies set forth above. In yet another embodiment, the EGFR inhibitor administered to the subject comprises a small molecule inhibitor of EGFR signaling, such as one or more of the small molecule inhibitors set forth above.

The compositions and methods provided herein can be used to inhibit growth, invasiveness or metastasis of a tumor, or treat a subject carrying a tumor that is resistant toErbB pathway inhibition. In one embodiment, the tumor is a lung cancer tumor, such as a non-small cell lung cancer (NSCLC) tumor, e.g., an adenocarcinoma NSCLC, a squamous cell carcinoma NSCLC, or a large cell carcinoma NSCLC. In other embodiments, the tumor can be a colorectal cancer, head and neck cancer, pancreatic cancer or breast cancer tumor.

In another aspect, pharmaceutical compositions for overcoming resistance to an ErbB pathway inhibitor are provided. The pharmaceutical compositions comprise one or more of an ErbB3 inhibitor as described above and an ErbB pathway inhibitor as described above. In one embodiment, the ErbB3 inhibitor and the ErbB pathway inhibitor are formulated with a pharmaceutical carrier into a single composition. In another embodiment, the ErbB3 inhibitor is formulated with a first pharmaceutical carrier to form a first composition, the ErbB pathway inhibitor is formulated with a second pharmaceutical carrier to form a second composition and the first and second composition are optionally packaged together.

### BRIEF DESCRIPTION OF THE DRAWINGS

FIG. 1 is a series of graphs of ELISA assay results, showing inhibition of heregulin-induced phosphorylation of ErbB3 (pErbB3), AKT (pAKT) and ERK (pERK) in the ACHN, DU145 and OvCAR8 cell lines by the anti-ErbB3 antibody MM-121. Data represent the mean±SD of two separate experiments.

FIG. 2 is a series of graphs showing spheroid assays using EGFR wild-type NSCLC cell lines. Spheroid cell cultures were treated with EGF (10 nM), heregulin1-β1 (HRG) (10 nM), both EGF and HRG (10 nM), or no exogenous ligands, as well as with erlotinib (1 μM), MM-121 (1 μM), a combination of the two (1 μM each), or in the absence of drugs. Cell lines tested include adenocarcinoma cell lines NCI-H322M, FIG. 2A; EKVX, FIG. 2B; A549, FIG. 2C; H358, FIG. 2D, and squamous cell line SW-900, FIG. 2E. The y-axes represent relative live cell density.

FIG. 3 is a series of graphs showing spheroid assays using EGFR wild-type NSCLC cell lines. Spheroid cell cultures were treated with doses of erlotinib ranging from 0 to 10 μM, in either the absence or presence of heregulin-1β1(HRG), and in either the absence or presence of MM 121. Cells tested include the adenocarcinoma cell lines NCI-H322M (FIG. 3A); EKVX (FIG. 3B); A549 (FIG. 3C); NCI-H358 (FIG. 3D); NCI-H441 (FIG. 3E), NCI-H2347 (FIG. 3F); the squamous cell carcinoma cell line NCI-H2170 (FIG. 3G), and the large cell carcinoma cell line NCI-H661 (FIG. 3H). The y-axes represent relative live cell density.

FIG. 4 is a graph showing cell viability of cisplatin sensitive (A2780) and resistant (A2780cis) cells after treatment with cisplatin. Cell viability (Y-axis) is given as % viability of media control and is plotted against drug concentration in log μM.

FIG. 5 shows images of three western blots probed with anti-pAKT. Shown are pAKT levels in lysate from A2780 cells treated with cisplatin (FIG. 5A), A2780cis cells treated with cisplatin (FIG. 5B), and A2780cis cells treated with MM-121 (FIG. 5C). S indicates untreated sensitive cell control and R indicates untreated resistant cell control. The X-axes indicate the time the lysates were harvested in hours post drug treatment and the Y-axes indicate drug concentration in μM.

FIG. 6 comprises a series of graphs showing cell viability (as % control) of BT474-M3 cells in vitro (Y-axis) after treatment with: FIG. 6A, lapatinib, FIG. 6B trastuzumab, or FIG. 6C, MM-111. For each the X-axis indicates drug concentration in nM.

FIG. 7 comprises two graphs showing inhibition of AKT activation in heregulin-stimulated BT474-M3 cells (Y-axes, normalized amounts of pAKT) after treatment with: FIG. 7A lapatinib, FIG. 7B, trastuzumab. For each the X-axis shows ErbB pathway inhibitor concentrations as indicated. For each indicated lapatinib concentration along the X-axis in FIG. 7A the grouped bars, reading from left to right, correspond to MM-111 concentrations of 0 nM, 1 nM, 4 nM, 16 nM, 63 nM, 250 nM, and 1000 nM. For indicated trastuzumab concentration along the X-axis in FIG. 7B the grouped bars, reading from left to right, correspond to trastuzumab concentrations of 0 nM, 1 nM, 10 nM, 100 nM, and 1000 nM. The lines marked “basal” indicate the pAKT level in cells that were not stimulated with heregulin and were not treated with MM-111, lapatinib, or trastuzumab.

FIG. 8 comprises two graphs showing reductions in tumor volumes in a BT474-M3 xenograft breast cancer xenograft model after treatment with: FIG. 8A, MM-111, lapatinib, or a combination of MM-111 and lapatinib; FIG. 8B MM-111, trastuzumab, or a combination of MM-111 and trastuzumab. The Y-axes represent mean tumor volume in mm³ and the x-axes represent time in days post tumor implant.

FIG. 9 comprises three plots showing FACS analysis of ErbB receptors in trastuzumab-resistant BT474-M3 cells. For each, cell count (Y-axis) is plotted against fluorochrome emission intensity (X-axis) for: FIG. 9A, ErbB2 receptor status—thick solid black line=trastuzumab-resistant cells stained for ErbB2, thinner solid gray line=trastuzumab-resistant cells, unstained, dotted line=non-resistant cells, stained for ErbB2, dashed line=non-resistant cells, unstained; FIG. 9B, EGFR status—thick solid black line=trastuzumab-resistant cells stained for EGFR, thinner solid gray line=trastuzumab-resistant cells, unstained, dotted line=non-resistant cells, stained for EGFR, dashed line=non-resistant cells, unstained; FIG. 9C, ErbB3 status ErbB3 receptor status, thick solid black line=trastuzumab-resistant cells stained for ErbB3, thinner solid gray line=trastuzumab-resistant cells, unstained, dotted line=non-resistant cells, stained for ErbB3, dashed line=non-resistant cells, unstained.

FIG. 10 comprises two graphs comparing the ability of: FIG. 10A, trastuzumab and FIG. 10B, MM-111, to inhibit cell growth in trastuzumab-resistant BT474-M3 cells. For each, the growth of parental (non-resistant) and trastuzumab-resistant BT474-M3 cells is shown as % of control (no trastuzumab or MM-111 added) cells (Y-axis) after treatment with a dose range of trastuzumab (A) or MM-111 (B) (X-axes, in nM trastuzumab or MM-111).

FIG. 11 comprises two graphs comparing the ability of: FIG. 11A, trastuzumab, and FIG. 11B, MM-111, to inhibit cell growth in trastuzumab-resistant BT474-M3 cell spheroids. The growth of parental (non-resistant) and trastuzumab-resistant BT474-M3 cell spheroids is shown as % of control (no trastuzumab or MM-111 added) cells (Y-axes) after treatment with a dose range of drug (X-axes, in nM trastuzumab or MM-111).

FIG. 12 is two graphs comparing the effect of MM-111 or trastuzumab on cell growth of trastuzumab-resistant BT474-M3 cell spheroids when in combination with 300 nM erlotinib (FIG. 12A) or 100 nM gefitinib (FIG. 12B). The growth of the cell spheroids is shown as % of control (dashed line, no drug added). The growth of cell spheroids treated with erlotinib alone or gefitinib alone is shown by dot-dash lines. The x-axes are a log scale of each of MM-111 and/or trastuzumab concentration in nM and the y axis is spheroid size as % of control spheroid size.

FIG. 13 is a series of graphs showing the effect of MM-111, lapatinib, and tamoxifen on tumor growth inhibition in a BT474-M3 xenograft model. In FIGS. 13A, 13B, and 13C, the left panel shows tumor volume of xenografts of BT474-M3 cells that have been engineered to express green fluorescent protein and the right panel shows tumor volume in xenografts of the BT474-M3 cells that have been engineered to express GFP and heregulin 1. FIG. 13A shows the tumor growth curves of BT474-M3-GFP and BT474-M3-GFP-HRG tumors wherein the mice were treated with MM-111 (48 mpk), lapatinib (150 mpk) and tamoxifen (5 mg). FIG. 13B shows the tumor growth curves of BT474-M3-GFP and BT474-M3-GFP-HRG tumors wherein the mice were treated with MM-111 (48 mpk)+lapatinib (150 mpk), MM-111+tamoxifen (5 mg), and lapatinib+tamoxifen combination therapies. FIG. 13C shows the tumor growth curves of BT474-M3-GFP and BT474-M3-GFP-HRG tumors wherein the mice were treated with lapatinib+tamoxifen and MM-111+lapatinib+tamoxifen combination therapies. Control mice received no treatment. The x-axes are time in days and the y-axes are tumor volume in relation to the tumor volume at the start of treatment on day 17 or day 20 after inoculation (“Ratio to D17” or “Ratio to D20”).

FIG. 14 is a series of graphs showing the effect of MM-111, lapatinib and tamoxifen mono- and combination therapies on HRG-induced signaling in BT474-M3-GFP (left panels) and BT474-M3-GFP-HRG (right panels) tumors. FIG. 14A shows pErbB3 levels in pg/ml; FIG. 14B shows total ErbB3 levels (tErbB3) in pg/ml; FIG. 14C shows the ratio of phospho-ErbB3 to total ErbB3; FIG. 14D shows phospho-Akt (pAkt) levels in pg/ml; FIG. 14E shows total Akt levels (tAkt) in pg/ml; FIG. 14F shows the ratio of pAkt to tAkt; FIG. 14G shows phospho-ERK1/2 levels (pERK) normalized to the levels of proliferating cell nuclear antigen (PCNA); and FIG. 14H shows total ERK1/2 levels (totERK) normalized to the levels of PCNA; FIG. 14I shows the ratio of pERK and totERK levels. The x-axes represent the therapy the tumor-bearing mice received. Control mice received no treatment.

FIG. 15 is a series of graphs showing tumor growth curves in an NCI-N87 xenograft model. FIG. 15A shows tumor bearing mice either untreated (control) or treated with trastuzumab (3.5 mpk)+5-fluorouracil (5-FU; 12 mpk 5 days/week), trastuzumab+5-FU+cisplatin (5 mpk), 1^{st} line MM-111 (96 mpk)+trastuzumab+5-FU, 2^{nd} line MM-111+trastuzumab+5-FU and 2^{nd} line MM-111+trastuzumab+5-FU+cisplatin. The switch to 1^{st} to 2^{nd} line treatments as well as discontinuation of the chemotherapeutics is indicated with arrows. FIG. 15B shows the tumor growth curves of NCI-N87—tumors treated with trastuzumab+5-FU and 2^{nd} line MM-111+trastuzumab+5-FU. FIG. 15C shows the tumor growth curves of NCI-N87—tumors treated with trastuzumab+5-FU+cisplatin and 2^{nd} line MM-111+trastuzumab+5-FU+cisplatin. The x-axes are time in days and the y-axes are tumor volume in mm³.

FIG. 16 is a series of graphs showing the effect of MM-111, lapatinib and tamoxifen mono- and combination therapies on HRG-induced signaling in BT474-M3-GFP (left panels) and BT474-M3-GFP-HRG (right panels) tumors. Tumor-bearing mice were treated with MM-111, lapatinib and trastuzumab monotherapies (FIG. 16A); MM-111+lapatinib, MM-111+trastuzumab, and lapatinib+trastuzumab combination therapies (FIG. 16B); or lapatinib+trastuzumab and MM-111+lapatinib+trastuzumab combination therapies (FIG. 16C). The x-axes are time in days and the y-axes are tumor volume in relation to the tumor volume at the start of treatment on day 17 after inoculation (“Ratio”).

FIG. 17 is a graph showing tumor growth curves in an NCI-N87 xenograft model. Tumor bearing mice were either untreated (control) or treated with paclitaxel (20 mpk), trastuzumab (3.5 mpk)+paclitaxel, or MM-111 (48 mpk)+trastuzumab+paclitaxel. The x-axis is time in days and the y-axis is tumor volume in mm³.

### DETAILED DESCRIPTION

Methods for overcoming resistance to ErbB pathway inhibitors are provided, as well as pharmaceutical compositions that can be used in such methods. As described further in the Examples, it has now been demonstrated that an ErbB3 inhibitor, e.g., an anti-ErbB3 antibody or a bispecific anti-ErbB3, anti-ErbB2 antibody, is able to overcome ligand-induced (e.g., heregulin-induced) resistance to an ErbB pathway-targeted therapy (e.g., an EGFR-targeted therapy) both in vitro and in vivo. Accordingly, provided herein are methods for overcoming resistance to ErbB pathway inhibitors by combining the use of an ErbB pathway inhibitor with an ErbB3 inhibitor.

Although not intended to be limited by mechanism or bound by any theory of operation, the ability of the ErbB3 inhibitors described herein to overcome resistance to ErbB pathway inhibitors is thought to be due, at least in part, to the ability of the ErbB3 inhibitor to block ligand-dependent reactivation of ErbB3 signaling that leads to reactivation of PI3K/AKT signaling.

So that this disclosure may be more readily understood, certain terms are first defined.

As used herein, the term “inhibitor” indicates therapeutic agents that inhibit, downmodulate, suppress or downregulate activity of a receptor or other signal transduction protein, including signaling mediated thereby. The term encompasses small molecule inhibitors (e.g., small molecule tyrosine kinase inhibitors) and antibodies, interfering RNA (shRNA, siRNA), soluble receptors and the like.

An “ErbB pathway inhibitor” is an inhibitor that acts on one or more proteins of one or more ErbB signaling pathways, such as the EGFR (ErbB1/HER1) signaling pathway or the HER2 (ErbB2, neu) signaling pathway.

An “EGFR inhibitor” acts on EGFR.

A “HER2 inhibitor” acts on HER2 (ErbB2).

An “ErbB3 inhibitor” acts on ErbB3.

An “antibody” is a whole antibody or any antigen binding fragment (i.e., “antigen-binding portion”) or single chain thereof. The term “antibody” encompasses: (i) monoclonal antibodies; (ii) recombinant antibodies (i.e., antibodies that are prepared, expressed, created or isolated by recombinant means); (iii) chimeric antibodies (i.e., antibodies in which the variable domain(s) are from one species and the constant domain(s) are from another); (iv) humanized antibodies (i.e., antibodies in which only the CDRs are from a donor species and the rest of the antibody structure is human, although additional FR substitutions, either donor substitutions or non-donor/non-acceptor substitutions, may be incorporated); (v) fully human antibodies (i.e., antibodies in which the variable region CDRs and FRs are derived from human germline immunoglobulin sequences); and (vi) bispecific and multispecific antibodies (i.e., antibodies having two or more binding sites with different antigen-binding specificities). The term “antigen-binding portion” of an antibody (or simply “antibody portion”), as used herein, refers to one or more fragments of an antibody that retain the ability to specifically bind to an antigen (e.g., ErbB3). Examples of binding fragments encompassed within the term “antigen-binding portion” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) an Fv fragment consisting of the V_{L} and V_{H} domains of a single arm of an antibody, (v) a dAb including V_{H} and V_{L} domains; (vi) a dAb fragment, which consists of a V_{H} domain; (vii) a dAb which consists of a V_{H} or a V_{L} domain; and (viii) a combination of two or more isolated CDRs which are joined, e.g., by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form a monovalent molecule (known as a single chain Fv (scFv). Such single chain antibodies are also intended to be encompassed within the term “antigen-binding portion” of an antibody. These antibody fragments may be obtained using conventional techniques known to those with skill in the art, and the fragments may be screened for utility in the same manner as are intact antibodies. Antigen-binding portions can be produced by recombinant DNA techniques, or by enzymatic or chemical cleavage of intact immunoglobulins.

The term “resistance to an ErbB pathway inhibitor” (such as in “exhibiting resistance to an ErbB pathway inhibitor”) refers to the property of a cell (e.g., a cancer cell) in which the cell displays reduced, diminished or a lack of responsiveness to an ErbB pathway inhibitor (e.g., as measured by the ability of the inhibitor to inhibit cell growth or proliferation), as compared to the same cell at an earlier time point or as compared to other cells of the same type that respond to the ErbB pathway inhibitor. The cell can be within tissue, e.g., tumor tissue. Furthermore, the tissue, e.g., tumor tissue, can be in a subject, in which case the subject is referred to as exhibiting resistance to the ErbB pathway inhibitor.

As used herein, the term “acquired resistance” refers to resistance to an ErbB pathway inhibitor that develops in a cell over time, typically during the course of treatment with an ErbB pathway inhibitor, such that the responsiveness of the cell to the ErbB pathway inhibitor diminishes over time as compared to the responsiveness of such a cell to the inhibitor at the start of treatment. This “acquired resistance”, which develops over time in previously responsive cells, is in contrast to cells that are “predisposed to resistance” to an ErbB pathway inhibitor, which refers to an inherent lack of, or significantly reduced, responsiveness of the cells to treatment with an ErbB pathway inhibitor as compared to cells that exhibit responsiveness to (i.e., cells whose growth and/or proliferation is significantly inhibited by) the ErbB pathway inhibitor.

As used herein, the terms “overcoming resistance” and “overcome resistance” to an inhibitor, e.g., an ErbB pathway inhibitor, refers to the phenomenon in which the level or amount or degree of resistance to an inhibitor in a previously-resistant cell is diminished, reduced or reversed such that the cell exhibits a measurable degree of responsiveness (or increased responsiveness) to the same inhibitor, or another inhibitor that inhibits the same signaling pathway, as compared to the cell in its resistant state. For example, “overcoming resistance to an EGFR pathway inhibitor” in a cell, wherein the inhibitor for which resistance has been demonstrated is, for example, gefitinib, results in a cell that exhibits a measurable degree of responsiveness to gefitinib or to another EGFR pathway inhibitor (such as the anti-EGFR antibody cetuximab) as compared to the cell in its resistant state.

As used herein, the term “subject” includes any human or nonhuman mammal having a disease or disorder for which resistance to an ErbB pathway inhibitor can be addressed using a method provided herein, such as a subject or patient with a tumor exhibiting such resistance

I. ErbB3 Inhibitors

As described in further detail herein, the methods and compositions provided herein involve the use of one or more ErbB3 inhibitors.

MM-121 is a fully human anti-ErbB3 antibody currently undergoing Phase II clinical trials. MM-121 (also referred to as “Ab #6”) and related human anti-ErbB3 antibodies are described in detail in U.S. Pat. No. 7,846,440, U.S. Patent Publication Nos. 20090291085, 20100056761, and 20100266584, and PCT Publication No. WO 2008/100624. Other anti-ErbB3 antibodies that may be used in a disclosed combination include any of the other anti-ErbB3 antibodies described in U.S. Pat. No. 7,846,440, such as Ab #3, Ab #14, Ab #17 or Ab #19 or an antibody that competes with Ab #3, Ab #14, Ab #17 or Ab #19 for binding to ErbB3. Additional examples of anti-ErbB3 antibodies that may be administered in accordance with the methods disclosed herein include antibodies disclosed in U.S. Pat. No. 7,285,649, Patent Publications Nos. 20100310557, and 20100255010, as well as antibodies 1B4C3 (cat # sc-23865, Santa Cruz Biotechnology) and 2D1D12 (U3 Pharma AG), both of which are described in, e.g., U.S. Patent Publication No. 20040197332 and are produced by hybridoma cell lines DSM ACC 2527 or DSM ACC 2517 (deposited at DSMZ) anti-ErbB3 antibodies disclosed in U.S. Pat. No. 7,705,130 including but not limited to the anti-ErbB3 antibody referred to as AMG888 (U3-1287—U3 Pharma AG and Amgen), described in, e.g., U.S. Pat. No. 7,705,130; the anti-ErbB3 antibody referred to as AV-203 (Aveo Pharmaceuticals) which is described in U.S. Patent Publication No. 20110256154, the monoclonal antibodies (including humanized versions thereof), such as 8B8 (ATCC® HB-12070™), described in U.S. Pat. No. 5,968,511, and the anti-ErbB3 antibodies mAb 205.10 (e.g., mAb 205.10.1, mAb 205.10.2, or mAb 205.10.3) (Roche-Glycart), described in, e.g., U.S. Patent Publication No. 20110171222. Other such examples include anti-ErbB3 antibodies that are multi-specific antibodies and comprise at least one anti-ErbB3 antibody (e.g., one of the aforementioned anti-ErbB3 antibodies) linked to at least a second therapeutic antibody or to an additional therapeutic agent. Yet other suitable anti-ErbB3 antibodies comprise either: 1) variable heavy (VH) and/or variable light (VL) regions encoded by the nucleic acid sequences set forth in SEQ ID NOs:45 and 46, respectively, or 2) VH and/or VL regions comprising the amino acid sequences set forth in SEQ ID NOs: 1 and 2, respectively, or 3) CDRH1, CDRH2, and CDRH3 sequences comprising the amino acid sequences set forth in SEQ ID NO:3(CDRH1) SEQ ID NO:4(CDRH2) and SEQ ID NO: 5(CDRH3), and/or CDRL1, CDRL2, and CDRL3 sequences comprising the amino acid sequences set forth in SEQ ID NO: 6 (CDRL1) SEQ ID NO: 7 (CDRL2) and SEQ ID NO: 8 (CDRL3) as well as an antibody that binds to human ErbB3 and has at least 90% variable region sequence identity with the above-mentioned antibodies 1), 2), or 3). In another embodiment, the antibody competes for binding with and/or binds to the same epitope on human ErbB3 as any one of the above-mentioned antibodies. When the antibody is MM-121, the epitope typically comprises residues 92-104 of human ErbB3 (SEQ ID NO: 41). In other embodiments, the antibody is a fully human monoclonal antibody that binds to ErbB3 and, in living cells and either a) inhibits ErbB2/ErbB3 complex formation or b) prevents intracellular phosphorylation of ErbB3 induced by any of the forms of each of the following: heregulin, EGF, TGFα, betacellulin, heparin-binding epidermal growth factor, biregulin, epigen, epiregulin, and amphiregulin, or does both a) and b).

Anti-ErbB3 antibodies described above, can be generated, e.g., in prokaryotic or eukaryotic cells, using methods well known in the art, e.g., in a cell line capable of glycosylating proteins, such as CHO cells.

MM-111 (also referred to as B2B3-1), is described in co-pending U.S. patent application Ser. No. 12/757,801, and PCT Publication No. WO2009/126920. Also disclosed therein are other bispecific anti-ErbB2/antiErbB3 antibodies that are scFv HSA conjugates and that are suitable for use in the methods and compositions provided herein, including A5-HSA-ML3.9, A5-HSA-B1D2, B12-HSA-B1D2, A5-HSA-F5B6H2, H3-HSA-F5B6H2, and F4-HSA-F5B6H2. Other bispecific anti-ErbB2/antiErbB3 antibodies that are suitable for use in the methods and compositions provided herein include ALM and H3×B1D2, each comprising an anti-ErbB3 antibody linked to an anti-ErbB2 antibody, which are described further in U.S. Pat. Nos. 7,332,585 and 7,332,580, as well as in PCT Application Serial No. PCT/US2007/024287.

In yet another embodiment, the bispecific anti-ErbB3, anti-ErbB2 antibody can comprise a mixture, or cocktail, of two or more bispecific anti-ErbB3, anti-ErbB2 antibodies, each of which binds to a different epitope on ErbB3. In one embodiment, the mixture, or cocktail, comprises three bispecific anti-ErbB3, anti-ErbB2 antibodies, each of which binds to a different epitope on ErbB3.

In another embodiment, the ErbB3 inhibitor comprises a nucleic acid molecule, such as an RNA molecule, that inhibits the expression or activity of ErbB3. RNA antagonists of ErbB3 have been described (see e.g., U.S. Patent Publication No. 20080318894). Moreover, interfering RNAs specific for ErbB3, such as shRNAs or siRNAs that specifically inhibit the expression and/or activity of ErbB3, have been described (see e.g., Sergina, N. V. et al. (2007) Nature 445:437-441; Liu, B. et al. (2007) Int. J. Cancer 120:1874-1882; Frolov, A. et al. (2007) Cancer Biol. Ther. 6:548-554; Sithanandam, G. and Anderson, L. M. (2008) Cancer Gene Ther. 15:413-418; Lee-Hoeflich, S. J. et al. (2008) Cancer Res. 68:5878-5887).

In yet another embodiment, the ErbB3 inhibitor comprises a soluble form of the ErbB3 receptor that inhibits signaling through the ErbB3 pathway. Such soluble ErbB3 molecules have been described in the art (see e.g., U.S. Pat. No. 7,390,632, U.S. Patent Publication No. 20080274504 and U.S. Patent Publication No. 20080261270, each by Maihle et al., and U.S. Patent Publication No. 20080057064 by Zhou).

In another embodiment, the ErbB3 inhibitor comprises a nucleic acid molecule, such as an RNA molecule, that inhibits the expression or activity of ErbB3. RNA antagonists of ErbB3 have been described (see e.g., U.S. Patent Publication No. 20080318894). Moreover, interfering RNAs specific for ErbB3, such as shRNAs or siRNAs that specifically inhibit the expression and/or activity of ErbB3, have been described (see e.g., Sergina, N. V. et al. (2007) Nature 445:437-441; Liu, B. et al. (2007) Int. J. Cancer 120:1874-1882; Frolov, A. et al. (2007) Cancer Biol. Ther. 6:548-554; Sithanandam, G. and Anderson, L. M. (2008) Cancer Gene Ther. 15:413-418; Lee-Hoeflich, S. J. et al. (2008) Cancer Res. 68:5878-5887).

In yet another embodiment, the ErbB3 inhibitor comprises a soluble form of the ErbB3 receptor that inhibits signaling through the ErbB3 pathway. Such soluble ErbB3 molecules have been described in the art (see e.g., U.S. Pat. No. 7,390,632, U.S. Patent Publication No. 20080274504 and U.S. Patent Publication No. 20080261270, each by Maihle et al., and U.S. Patent Publication No. 20080057064 by Zhou).

II. ErbB Pathway Inhibitors

As described in further detail herein, the methods and compositions provided herein involve the use of one or more ErbB pathway inhibitors.

In one embodiment, the ErbB pathway inhibitor is an EGFR inhibitor (i.e., an inhibitor that inhibits EGFR and thereby inhibits EGFR pathway signaling).

In one embodiment, the EGFR inhibitor comprises an anti-EGFR antibody. One A useful anti-EGFR antibody is cetuximab (Erbitux®, ImClone). Other examples of anti-EGFR antibodies include MM-151 (further described in Bukhalid et al., co-pending commonly assigned U.S. Patent Application Ser. No. 61/504,633, filed on Jul. 5, 2011), Sym004 (Symphogen, Pederson et al., Cancer Research Jan. 15, 2010 70; 588, also see U.S. Pat. No. 7,887,805), matuzumab (EMD72000), panitumumab (Vectibix®; Amgen); nimotuzumab (TheraCIM) and mAb 806 (Mishima, K. et al. (2001) Cancer Res. 61:5349-5354).

In another embodiment, the EGFR inhibitor comprises a small molecule inhibitor of the EGFR signaling pathway, such as a tyrosine kinase inhibitor (TKI) that inhibits the EGFR signaling pathway. An example of a small molecule inhibitor of the EGFR signaling pathway is gefitinib (Iressa®, AstraZeneca and Teva). Other examples of small molecule inhibitors of EGFR include erlotinib HCL (OSI-774; Tarceva®; OSI Pharma), lapatinib (Tykerb®, GlaxoSmithKline), canertinib (PD183805; Pfizer), PKI-166 (Novartis); PD158780; pelitinib; and AG 1478 (4-(3-Chloroanillino)-6,7-dimethoxyquinazoline).

In another embodiment, the ErbB pathway inhibitor is a HER2 inhibitor (i.e., an inhibitor that inhibits HER2 pathway signaling).

In one embodiment, the HER2 inhibitor comprises an anti-HER2 antibody. An example of an anti-HER2 antibody is trastuzumab (Herceptin®). Herceptin is commercially available from Genentech, Inc. Another example of an anti-HER2 antibody is pertuzumab (Omnitarg®; Genentech).

In another embodiment, the HER2 inhibitor comprises a small molecule inhibitor of the HER2, such as a tyrosine kinase inhibitor (TKI) that inhibits HER2 signaling. Non-limiting examples of small molecule inhibitors of HER2 signaling include lapatinib (Tykerb®, GlaxoSmithKline), CI-1033 (PD 183805; Pfizer), PKI-166 (Novartis) and pelitinib EKB-569.

III. Methods

In one aspect, a method is provided for overcoming resistance to an ErbB pathway inhibitor in a subject, the method comprising:

selecting a subject exhibiting resistance to an ErbB pathway inhibitor; and

administering to the subject (i) an ErbB3 inhibitor and (ii) an ErbB pathway inhibitor.

The ErbB pathway inhibitor administered to the subject does not need to be the same ErbB pathway inhibitor to which the subject has been demonstrated to be resistant, although typically, the ErbB pathway inhibitor administered to the subject will be directed against the same ErbB pathway as the ErbB pathway inhibitor to which the subject has been demonstrated to be resistant. For example, a subject who exhibits resistance to the EGFR pathway inhibitor gefitinib may be co-administered an ErbB3 inhibitor and an EGFR inhibitor, which EGFR inhibitor can be, for example, gefitinib or the anti-EGFR antibody cetuximab.

The ErbB3 inhibitor and the ErbB pathway inhibitor can be co-administered to the subject or, alternatively, the ErbB3 inhibitor can be administered prior to administration of the ErbB pathway inhibitor. The ErbB3 inhibitor and the ErbB pathway inhibitor can be administered to the subject by any route suitable for the effective delivery of the inhibitor to the subject. For example, many small molecule inhibitors are suitable for oral administration. Antibodies and other biologic agents typically are administered intravenously, intraperitoneally or intramuscularly.

Identification of a subject exhibiting resistance to an ErbB pathway inhibitor can be achieved through standard methods well known in the art. For example, the inability (or reduced ability) of the ErbB pathway inhibitor to inhibit the growth and/or proliferation of tumor cells in the subject (or a sample of tumor cells from the subject cultured in vitro) can be indicative of resistance.

In some embodiments, the resistance to the ErbB pathway inhibitor is acquired resistance, e.g., acquired resistance to an EGFR inhibitor, e.g., acquired resistance to gefitinib. In another embodiment, the acquired resistance to an EGFR inhibitor is associated with a T790M EGFR mutation in the subject, e.g., in a tumor in the subject. In another embodiment, the acquired resistance is to a HER2 inhibitor.

The resistance exhibited by the subject may be resistance associated with reactivation of PI3K/AKT signaling and the ErbB3 inhibitors inhibit PI3K/AKT signaling. Methods for assessing the ability of an ErbB3 inhibitor to inhibit PI3K/AKT signaling are known in the art, such as the assays described in detail in the Examples. For example, the ability of the ErbB3 inhibitor to inhibit phosphorylation of AKT can be assessed using standard techniques known in the art.

In another embodiment, the ErbB3 inhibitor administered to the subject is an anti-ErbB3 antibody. A useful anti-ErbB3 antibody comprises MM-121, comprising V_{H} and V_{L} sequences as shown in SEQ ID NOs: 1 and 2, respectively, or an antibody comprising VH CDR1, 2 and 3 sequences as shown in SEQ ID NOs: 3-5, respectively, and V_{L} CDR1, 2 and 3 sequences as shown in SEQ ID NOs: 6-8, respectively (i.e., the V_{H} and V_{L} CDRs of MM-121). In another embodiment, the anti-ErbB3 antibody is an antibody having heavy and light chains comprising the amino acid sequences set forth in SEQ ID NOs 42 and 43, respectively. Additional non-limiting exemplary anti-ErbB3 antibodies and other forms of ErbB3 inhibitors are described in detail in Subsection I above.

In another embodiment, the ErbB pathway inhibitor administered to the subject is an EGFR inhibitor. A useful EGFR inhibitor is an anti-EGFR antibody, e.g., cetuximab. Additional non-limiting exemplary anti-EGFR antibodies are described in detail in Subsection II above.

In another embodiment, the ErbB pathway inhibitor administered to the subject is a small molecule inhibitor of EGFR signaling as described in Subsection II above. A useful small molecule inhibitor of EGFR signaling is gefitinib. Additional non-limiting exemplary small molecule inhibitors of EGFR signaling are described in detail in Subsection II above.

In yet another embodiment, the ErbB pathway inhibitor administered to the subject is a HER2 inhibitor. Useful HER2 inhibitors include lapatinib and anti-HER2 antibodies, e.g., trastuzumab. Additional non-limiting exemplary anti-HER2 inhibitors are described in detail in Subsection II above.

In another aspect, a method of inhibiting growth, invasiveness or metastasis of a tumor is provided, wherein the tumor comprises cells comprising a T790M EGFR mutation, the method comprising contacting the tumor (or cells thereof) with (i) an EGFR inhibitor; and (ii) an ErbB3 inhibitor.

In certain embodiments, the ErbB3 inhibitor inhibits PI3K/AKT signaling. Methods for assessing the ability of an ErbB3 inhibitor to inhibit PI3K/AKT signaling are known in the art, such as the assays described in detail in the Examples. For example, the ability of the ErbB3 inhibitor to inhibit phosphorylation of AKT can be assessed using standard techniques known in the art.

In one embodiment, the tumor (or cells thereof) is contacted with an ErbB3 inhibitor comprising an anti-ErbB3 antibody as described in detail in Subsection I above.

In another embodiment, the tumor (or tumor cells) is contacted with an EGFR inhibitor comprising an anti-EGFR antibody as described in detail in Subsection II above. In another embodiment, the tumor (or tumor cells) is contacted with an EGFR inhibitor comprising a small molecule inhibitor of EGFR signaling, as described in detail in Subsection II above.

In yet another aspect, a method of treating a tumor in a subject is provided, the method comprising

selecting a subject with a tumor comprising a T790M EGFR mutation, and

administering to the subject (i) an EGFR inhibitor; and (ii) an ErbB3 inhibitor.

The ErbB3 inhibitor and the EGFR inhibitor can be co-administered to the subject or, alternatively, the ErbB3 inhibitor can be administered prior to administration of the EGFR inhibitor. The ErbB3 inhibitor and the EGFR inhibitor can be administered to the subject by any route suitable for the effective delivery of the inhibitor to the subject. For example, many small molecule inhibitors are suitable for oral administration. Antibodies and other biologic agents typically are administered intravenously, intraperitoneally or intramuscularly.

Identification of a subject with a tumor comprising a T790M EGFR mutation can be achieved using methods well known in the art, such as by analysis (e.g., by PCR and sequencing) of genomic DNA in, or cDNA encoding EGFR prepared from, a sample of tumor cells (e.g., a biopsy) from the subject.

A useful ErbB3 inhibitor is one that inhibits PI3K/AKT signaling. Methods for assessing the ability of an ErbB3 inhibitor to inhibit PI3K/AKT signaling are well known in the art, such as the assays described in detail in the Examples.

The ErbB3 inhibitor administered to the subject may comprise an anti-ErbB3 antibody, e.g., MM-121, which comprises V_{H} and V_{L} sequences as shown in SEQ ID NOs: 1 and 2, respectively, or the ErbB3 inhibitor may be an antibody comprising V_{H} CDR1, 2 and 3 sequences as shown in SEQ ID NOs: 3-5, respectively, and V_{L} CDR1, 2 and 3 sequences as shown in SEQ ID NOs: 6-8, respectively (i.e., the V_{H} and V_{L} CDRs of MM-121). Additional non-limiting exemplary anti-ErbB3 antibodies and other forms of ErbB3 inhibitors are described in detail in Subsection I above.

In another embodiment, the EGFR inhibitor administered to the subject comprises an anti-EGFR antibody as described in detail in Subsection II above. In another embodiment, the EGFR inhibitor administered to the subject comprises a small molecule inhibitor of EGFR signaling as described in detail in Subsection II above.

The methods disclosed herein are suitable for use in overcoming resistance to ErbB pathway inhibitors in essentially any diseases or disorders in which such resistance is observed, although the disease for which the methods are used is typically a cancer. In situations in which the resistance is to an EGFR inhibitor, the cancer typically is selected from the group consisting of lung cancer (e.g., non-small cell lung cancer), colorectal cancer, head and neck cancer and pancreatic cancer. In situations in which the resistance is to a HER2 inhibitor, the cancer typically is breast cancer.

IV. Pharmaceutical Compositions

In another aspect, pharmaceutical compositions are provided that can be used in the methods provided herein, i.e., pharmaceutical compositions for overcoming resistance to an ErbB pathway inhibitor. The pharmaceutical compositions typically comprise an ErbB3 inhibitor (as described in detail in Section I above), an ErbB pathway inhibitor (as described in detail in section II above) and a pharmaceutical carrier. The ErbB3 inhibitor and the ErbB pathway inhibitor can be formulated with the pharmaceutical carrier into a single composition. Alternatively, the ErbB3 inhibitor can be formulated with a pharmaceutical carrier to form a first composition, the ErbB pathway inhibitor can be formulated with a pharmaceutical carrier to form a second composition, and the first and second composition can be packaged together. Additionally, the pharmaceutical composition can include, for example, instructions for use of the composition to overcome resistance to an ErbB pathway inhibitor.

As used herein, “pharmaceutically acceptable carrier” includes any and all solvents, dispersion media, coatings, antibacterial and antifungal agents, isotonic and absorption delaying agents, buffers, and other excipients that are physiologically compatible. Preferably, the carrier is suitable for parenteral, oral, or topical administration. Depending on the route of administration, the active compound, e.g., small molecule or biologic agent, may be coated in a material to protect the compound from the action of acids and other natural conditions that may inactivate the compound.

Pharmaceutically acceptable carriers include sterile aqueous solutions or dispersions and sterile powders for the extemporaneous preparation of sterile injectable solutions or dispersion, as well as conventional excipients for the preparation of tablets, pills, capsules and the like. The use of such media and agents for the formulation of pharmaceutically active substances is known in the art. Except insofar as any conventional media or agents are incompatible with the active compound, use thereof in the pharmaceutical compositions disclosed herein is contemplated. Supplementary active compounds can also be incorporated into the compositions.

A pharmaceutically acceptable carrier can include a pharmaceutically acceptable antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers which may be employed in the pharmaceutical compositions disclosed herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be useful to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

These compositions may also contain functional excipients such as preservatives, wetting agents, emulsifying agents and dispersing agents.

Therapeutic compositions typically must be sterile, non-pyrogenic, and stable under the conditions of manufacture and storage. The composition can be formulated as a solution, microemulsion, liposome, or other ordered structure suitable to high drug concentration.

Sterile injectable solutions can be prepared by incorporating the active compound in the required amount in an appropriate solvent with one or a combination of ingredients enumerated above, as required, followed by sterilization, e.g., by microfiltration. Generally, dispersions are prepared by incorporating the active compound into a sterile vehicle that contains a basic dispersion medium and the required other ingredients from those enumerated above. In the case of sterile powders for the preparation of sterile injectable solutions, exemplary methods of preparation are vacuum drying and freeze-drying (lyophilization) that yield a powder of the active ingredient plus any additional desired ingredient from a previously sterile-filtered solution thereof.

Prevention of presence of microorganisms may be ensured both by sterilization procedures, supra, and by the inclusion of various antibacterial and antifungal agents, for example, paraben, chlorobutanol, phenol sorbic acid, and the like.

Pharmaceutical compositions disclosed herein can also be administered combination with other agents besides the ErbB3 inhibitors and ErbB pathway inhibitors described herein. For example, the combination therapy can include a combination of an ErbB3 inhibitor and an ErbB pathway inhibitor and at least one or more additional therapeutic agents, such as one or more chemotherapeutic agents known in the art. The pharmaceutical compositions and combinations thereof disclosed herein can also be administered in conjunction with radiation therapy and/or surgery.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation.

It is especially advantageous to formulate parenteral compositions disclosed herein in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions provided herein may be varied so as to obtain an amount of the active ingredient which is effective to achieve the desired therapeutic response for a particular patient, composition, and mode of administration, without being toxic to the patient.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The active compound may be mixed under sterile conditions with a pharmaceutically acceptable carrier, and with any preservatives, buffers, or propellants which may be required.

When compounds provided herein are administered as pharmaceuticals, to humans or animals, they can be given alone or as a pharmaceutical composition containing, for example, 0.001 to 90% (e.g., 0.005 to 70% or 0.01 to 30%) of active ingredient in combination with a pharmaceutically acceptable carrier.

The following examples should not be construed as further limiting the above disclosure. Each and every patent application and publication and issued patent cited herein is incorporated herein by reference in its entirety.

### EXAMPLES

### Part I: Use of Anti-ErbB3 Antibodies for Overcoming Resistance to ErbB Pathway Inhibitors

### Example 1

### MM-121 Blocks Ligand Induced Activation of ErbB3

In this example, the ability of the anti-ErbB3 monoclonal antibody MM-121 (Ab #6, as disclosed in U.S. Pat. No. 7,846,440) to inhibit ligand-induced activation of ErbB3 phosphorylation and signaling was examined in a series of in vitro experiments. In Vitro Signaling Studies Measured by ELISA

In the first set of experiments, three cancer cell lines (ACHN (renal cell adenocarcinoma, ATCC® #CRL-1611™), Du145 (prostate carcinoma, ATCC® #HTB-81™) and OvCAR 8 (ovarian adenocarcinoma, ATCC® #HTB-161™) cell lines; obtained from the National Cancer Institute's Developmental Therapeutics Program) were seeded at 35,000 cells per well in 96-well plates and grown overnight (maintenance culture medium was RPMI-1640 media supplemented with 10% fetal calf serum, 2 mM L-glutamine, and Pen-Strep; cells were grown in a humidified atmosphere at 5% CO₂, 95% air at 37° C.). Cells were synchronized by 20-24 hour serum starvation. The cells were then pre-incubated with 4-fold serial dilutions, ranging from 2 mM to 7.6 pM, of MM-121 for 30 minutes. The cells then were stimulated with 25 nM heregulin (HRG)-1beta for 10 minutes, washed once with cold PBS and lysed in MPER lysis buffer (Pierce Chemical Co.).

For ELISA analysis of cell lysates, capture antibodies against ErbB1 (R&D Systems AF231), ErbB2 (R&D Systems, MAB1129), ErbB3 (R&D Systems, MAB 3481) and AKT (Upstate, 05-591 MG) were incubated in 384 well black flat-bottom polystyrene high-binding plates (Corning, 3708) overnight at room temperature. ELISAs were blocked with 2% bovine serum albumin (BSA) in phosphate buffered saline (PBS) for one hour and then incubated with lysates diluted in 2% BSA, 0.1% Tween-20 and PBS for two hours at room temperature. Between incubation steps, plates were washed three times with 0.5% Tween-20 in PBS. ELISAs measuring phospho-ErbB1, -ErbB2 and -ErbB3 were incubated with phosphor-tyrosine horseradish peroxidase (HRP) linked monoclonal antibody (R&D Systems, HAM1676) for two hours. ELISAs measuring phosphor-AKT were incubated with primary serine 473 specific anti-phospho AKT mouse monoclonal antibody (Cell Signaling Technologies, 5102) for two hours, then incubated with streptavidin-HRP (DY998) for 30 minutes. All ELISAs were visualized with SuperSignal® ELISA Pico Chemiluminescent Substrate (Pierce, Cat. #37069) and luminescent signal was measured using a luminometer.

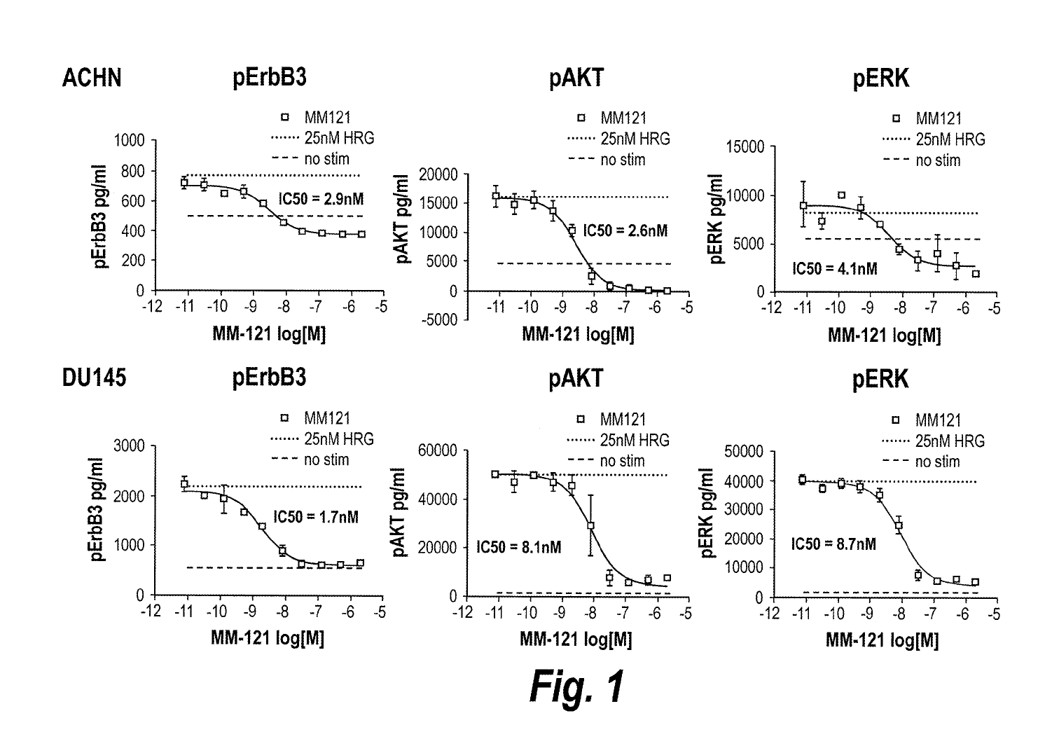

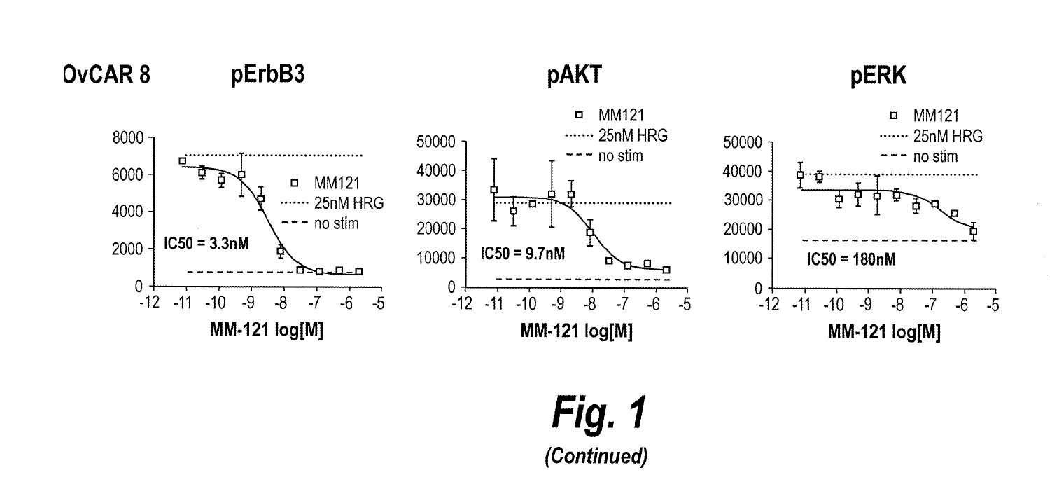

ELISA analysis of the cell lysates is summarized in the graphs shown in FIG. 1. The data demonstrate that MM-121 inhibits HRG-induced ErbB3, AKT and ERK phosphorylation in comparison to the 25 nM HRG control without MM-121. Inhibitor IC₅₀ values were calculated by least-squares fitting the dose response data with a sigmoidal curve (GraphPad Prism®, GraphPad Software, Inc., La Jolla, Calif.). In most cases, maximal inhibition of pErbB3, pAKT and pERK with MM-121 occurred near or below basal signaling levels as measured by the unstimulated control cells. Thus, the results of FIG. 1 demonstrate that in ACHN, Du145 and OvCAR8 cells, MM-121 blocked the capacity of heregulin to stimulate ErbB3 and downstream AKT and ERK phosphorylation and reduced signaling levels to equal or below the unstimulated cells.

### Example 2

### MM-121 Overcomes Resistance to Erlotinib in In-Vitro Models of EGFR-Wild-Type Non-Small Cell Lung Cancer Methods

Nine non-small cell lung cancer (NSCLC) cell lines were obtained from the American Type Culture collection: A549 (ATCC® #CCL-185™), EKVX (NCI vial 0502454), NCI-H2170 (ATCC® #CRL-5928™), NCI-H2347 (ATCC® #CRL-5942®), NCI-H322M (ATCC® # CRL-5806™), NCI-H358 (ATCC® # CRL-5806™), NCI-H441 (ATCC® #HTB-174™), NCI-H661 (ATCC® #HTB-183™) and SW-900 (ATCC® #HTB-59™). The cell lines bear no mutations within their EGFR genes and represent three distinct histological subtypes (see Table X below). Cells harboring Ras mutations are indicated.

Cells were seeded at 5,000 cells per well in a 96-well 3D-culture system (low-binding NanoCulture® plates, Scivax Corporation) and grown in RPMI-1640 medium supplemented with 10% fetal bovine serum and Pen-Strep at 37° C. 48 h later, after which time spheroids had formed, serum was reduced to 2% and cells were incubated with ligands and drugs for 6 days at 37° C. Relative live cell densities were then determined using the CellTiter Glo® reagent (Promega).

Two sets of experiments were performed: in one set, cells were treated with EGF (10 nM), heregulin1-β1 (HRG, 10 nM), a pool of the two ligands (10 nM each ligand), or in the absence of exogenous ligands, as well as with erlotinib (1 μM), MM-121 (1 μM), a combination of the two drugs (1 μM each drug), or in the absence of drugs. In a second set of experiments, cells were treated with doses of erlotinib ranging from 0 to 10 μM, in either the absence or presence of 10 nM heregulin −1β1, and in either the absence or presence of 1 μM MM 121.

TABLE 1
EGFR wild-type NSCLC cell lines

| | EGFR | K-Ras/H-Ras/N-Ras | |
|---|---|---|---|
| Cell Line | mutation status | mutation status | Histological subtype |
| A549 | wild-type | K-Ras G12S | adenocarcinoma |
| EKVX | wild-type | WT | adenocarcinoma |
| H2170 | wild-type | WT | squamous cell |
| | | | carcinoma |
| H2347 | wild-type | N-Ras Q61R | adenocarcinoma |
| H322M | wild-type | WT | adenocarcinoma |
| H358 | wild-type | K-Ras G12C | adenocarcinoma |
| H441 | wild-type | K-Ras G12V | adenocarcinoma |
| H661 | wild-type | WT | large cell carcinoma |
| SW-900 | wild-type | K-Ras G12V | squamous cell |
| | | | carcinoma |

### Results

Erlotinib (Tarceva®) is indicated for treatment of patients with non-small cell lung cancer and has been shown to be effective in patients with tumors harboring EGFR mutations; however, patients with tumors having wild-type EGFR show poor response rates to erlotinib in the clinic, and some patients on EGFR therapy develop resistance due to a new T790M mutation appearing (Hammerman et al., (2009) Clinical Cancer Research 15(24), 7502-7509. In vitro, EGFR wild-type NSCLC cell lines are substantially less sensitive to erlotinib, having GI₅₀'s several orders of magnitude higher than cell lines bearing EGFR-activating mutations. Activating mutations in Ras oncogenes (H-, N-, and K-Ras) have also been shown to be associated with lung cancer, particularly K-Ras mutations in lung adenocarcinomas. NSCLC subtypes other than adenocarcinoma are also found to correlate with poor response to erlotinib in clinical trials.

In order to demonstrate the effectiveness of MM-121 in overcoming resistance to erlotinib in EGFR wild-type NSCLC cells having multiple histological subtypes, MM-121 was tested in nine EGFR wild-type NSCLC cell lines having adenocarcinoma, squamous cell carcinoma, or large cell carcinoma histological subtypes. The cell lines are either wild-type for the Ras oncogene or harbor K-Ras or N-Ras mutations as indicated in Table 1.

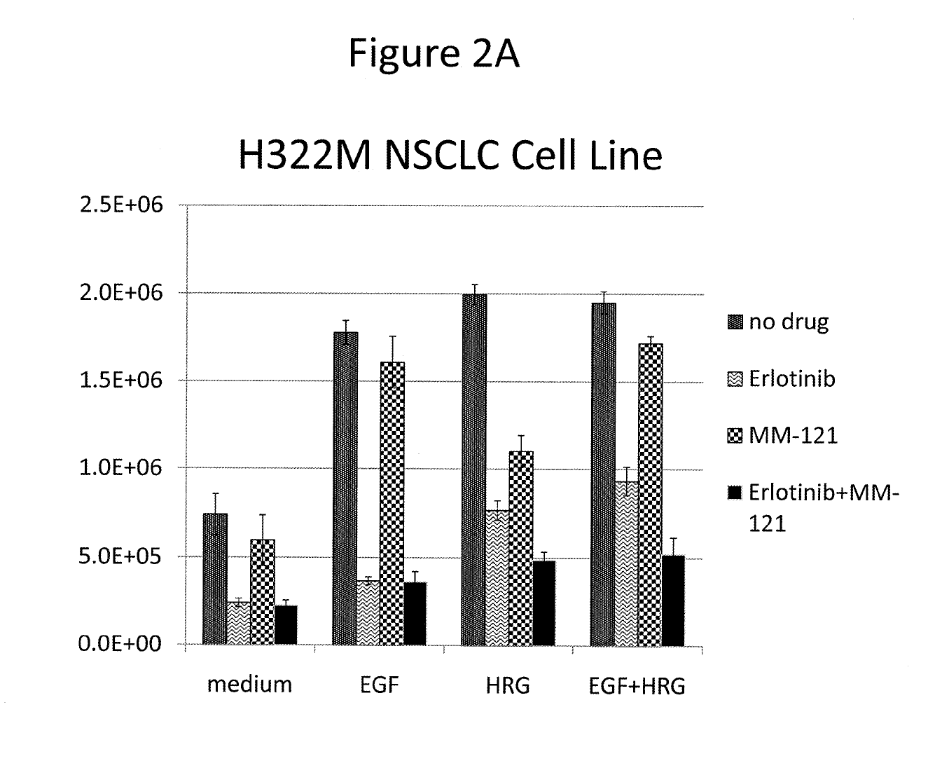

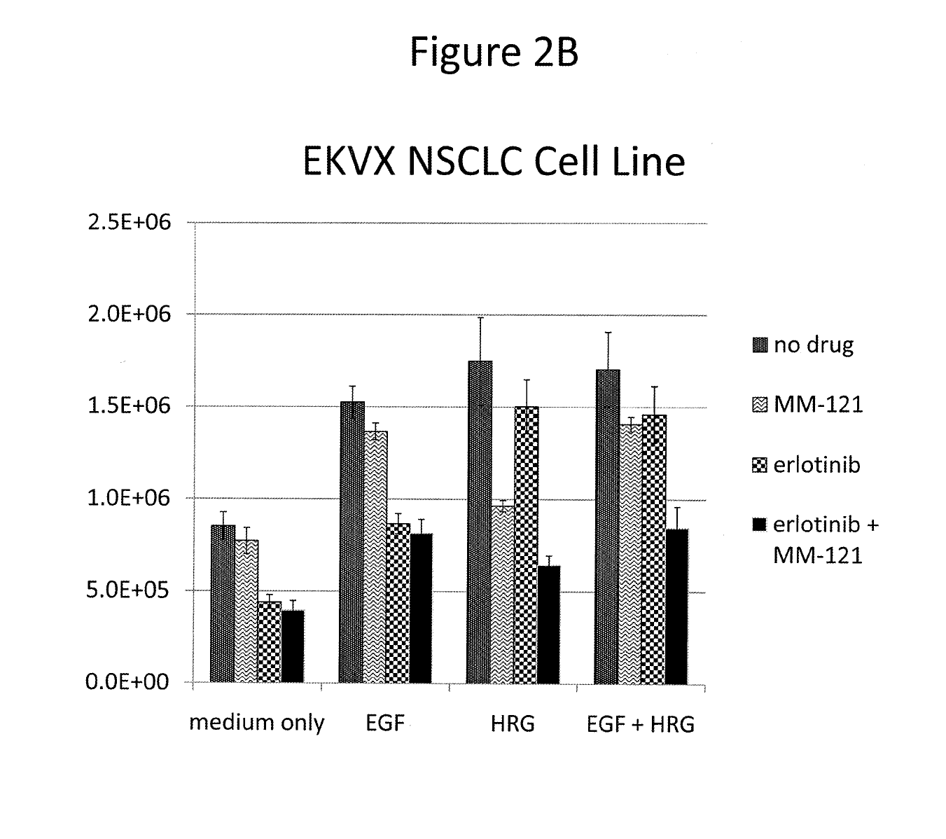

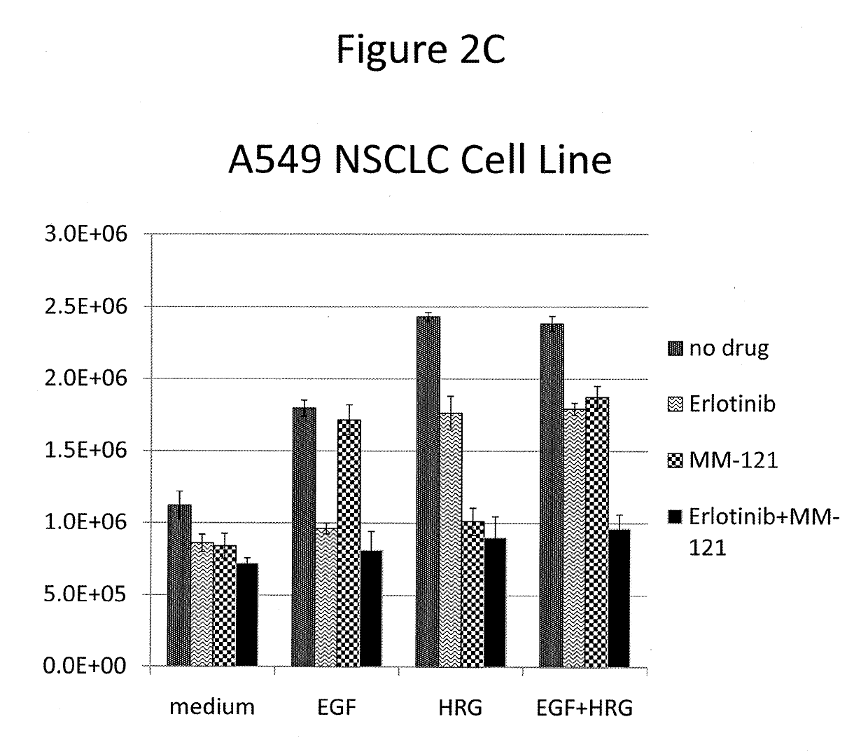

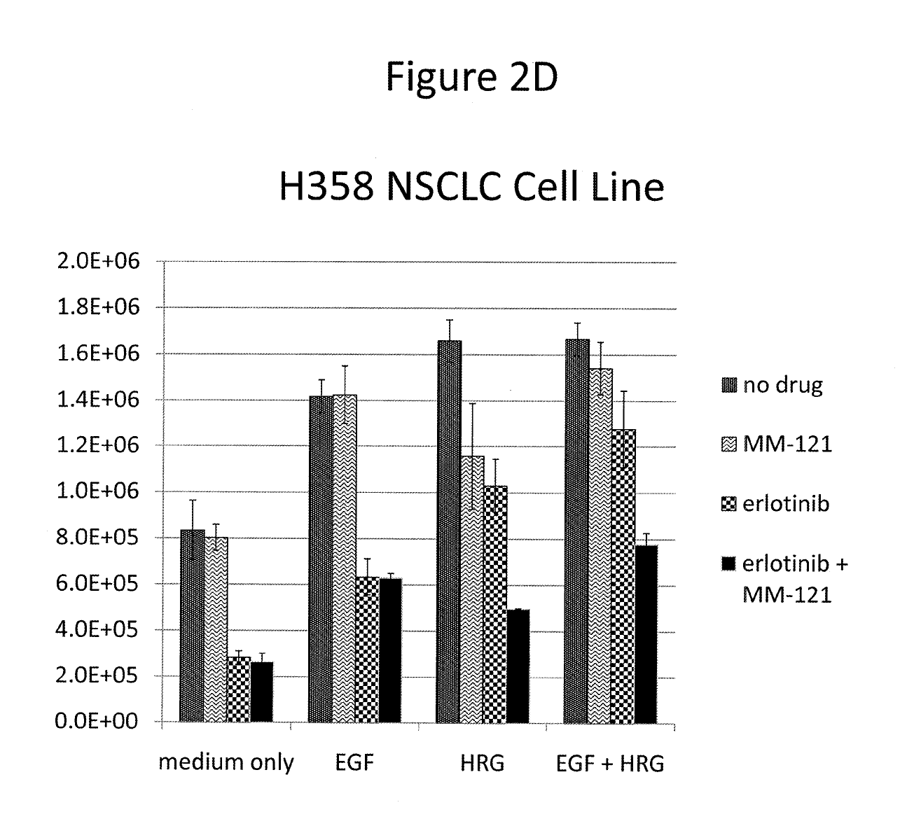

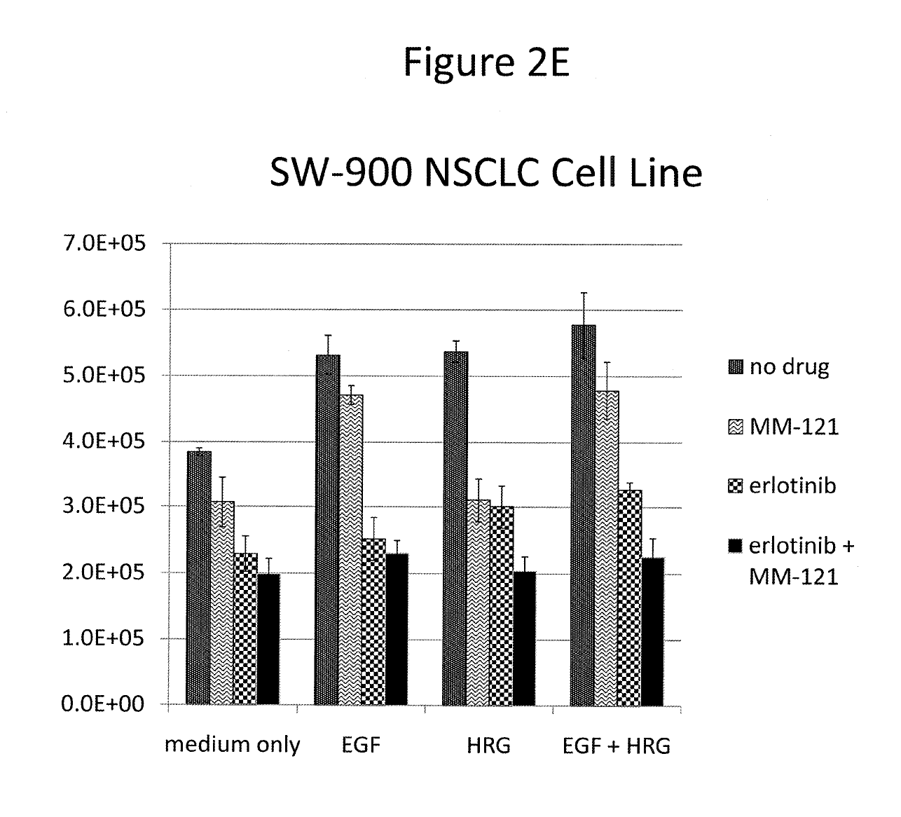

Five cell lines were each grown as spheroids and treated as described above with epidermal growth factor (EGF), heregulin1-β1 (HRG), a pool of ligands (EGF+HRG), or no exogenous ligands (medium), as well as with erlotinib, MM-121, a combination of erlotinib and MM-121, or no drug treatment. Tumor size was then determined by measuring live cell density. As shown in FIG. 2, erlotinib inhibited cell growth in the absence of exogenous ligands and in the presence of EGF, but was less effective at inhibiting growth of cells grown in the presence of HRG. While the combination of MM-121 and erlotinib was in some cell lines more effective at inhibiting cell growth in the EGF-stimulated cells, MM-121 was able to greatly improve sensitivity of the cells to erlotinib in HRG-stimulated cells. This was true for all cell spheroids tested, including adenocarcinoma cell lines NCI-H322M, FIG. 2A; EKVX, FIG. 2B; A549, FIG. 2C; H358, FIG. 2D, and squamous cell line SW-900, FIG. 2E. The mutation state of the K-Ras gene did not impact the effectiveness of MM-121 in increasing sensitivity of the cells to erlotinib.

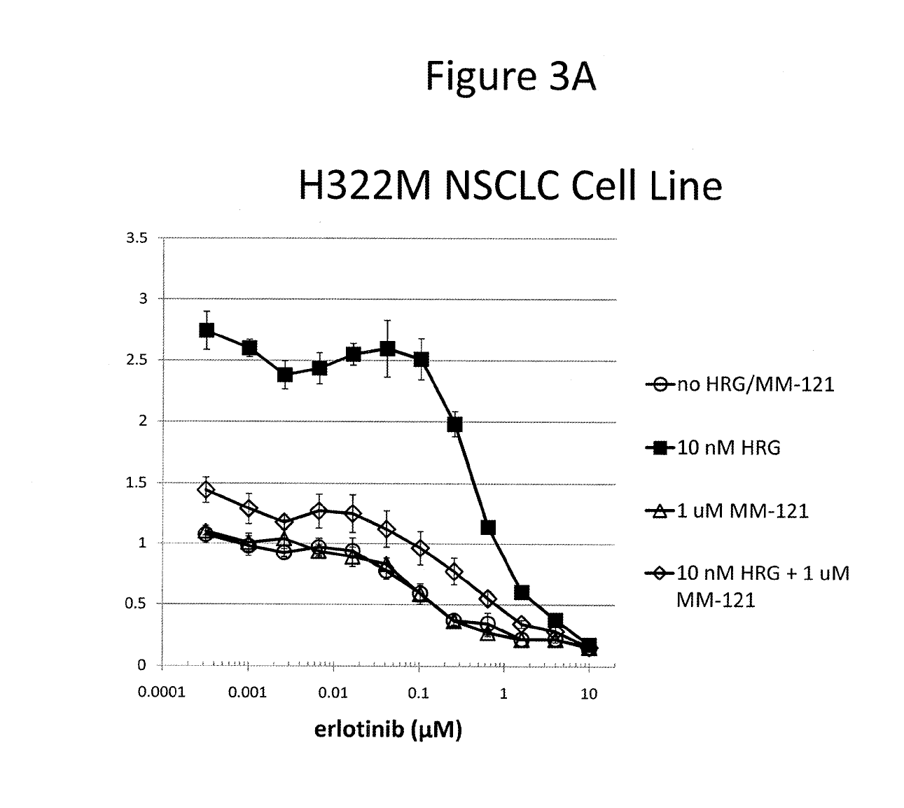

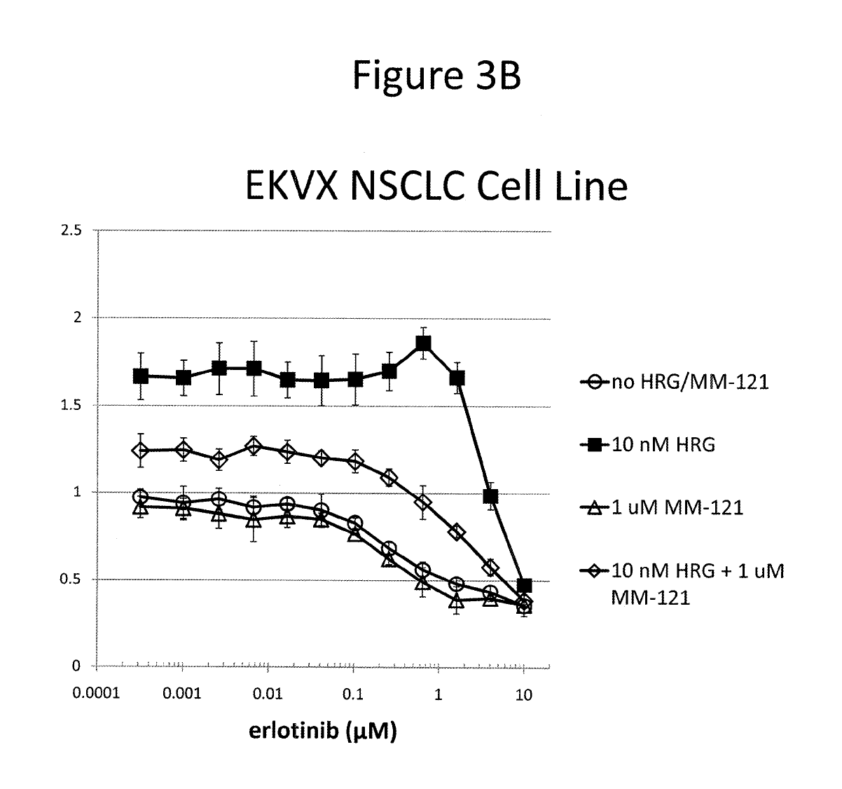

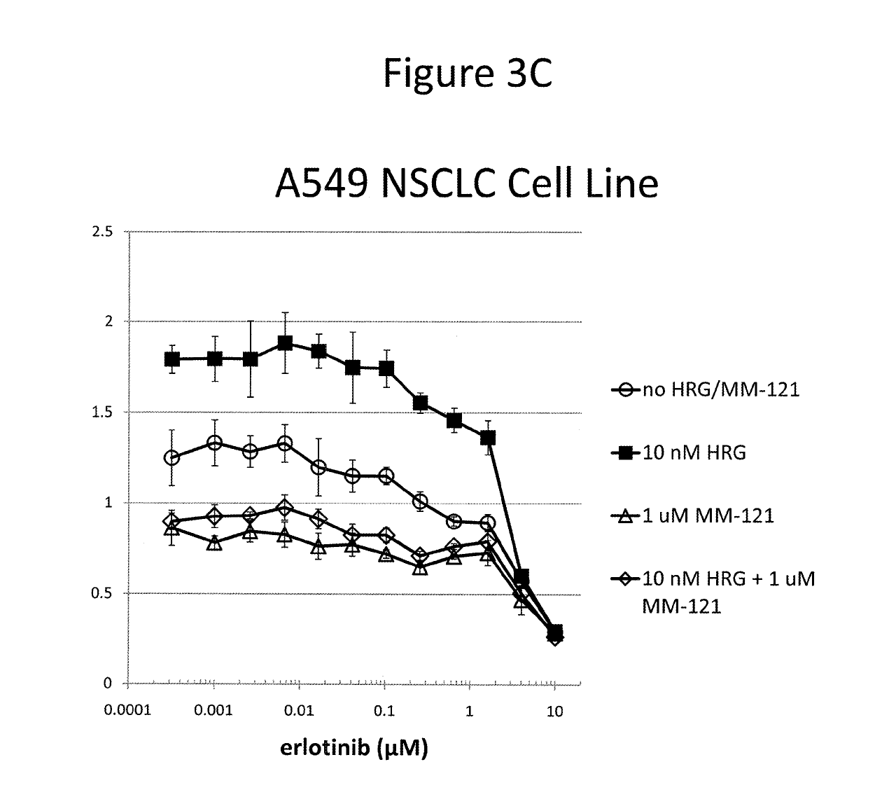

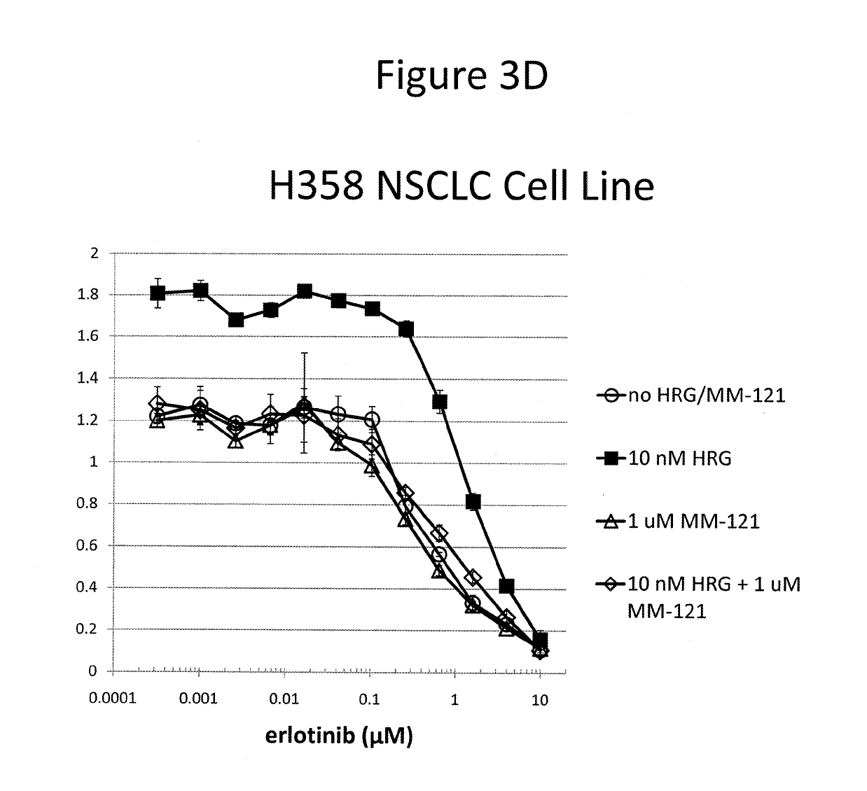

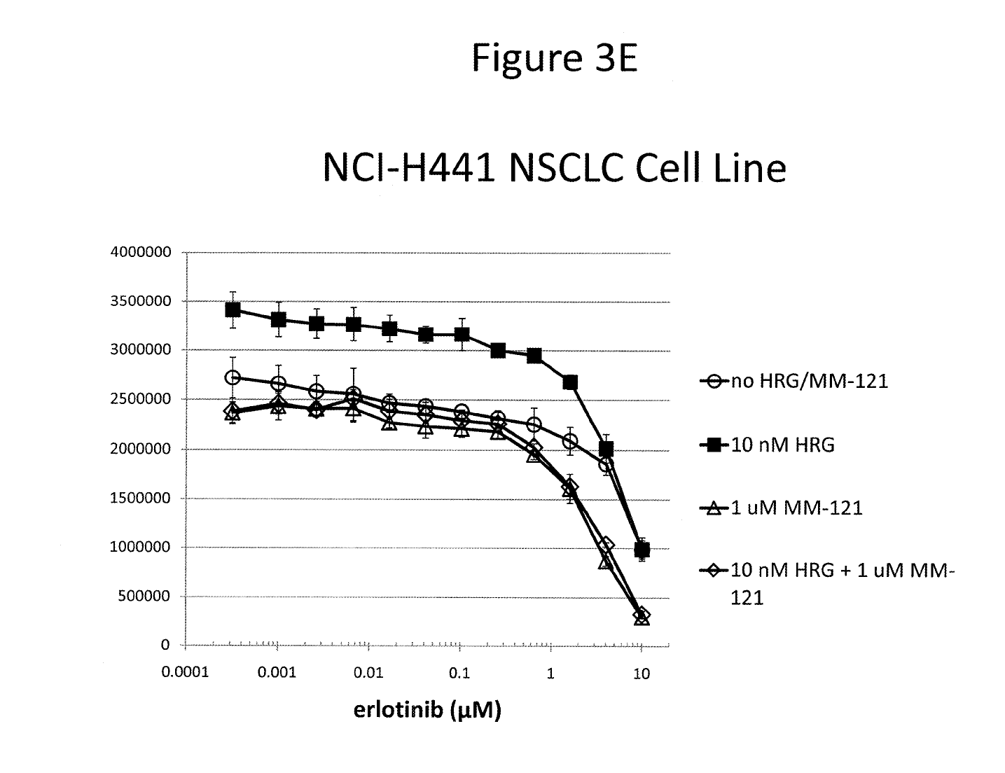

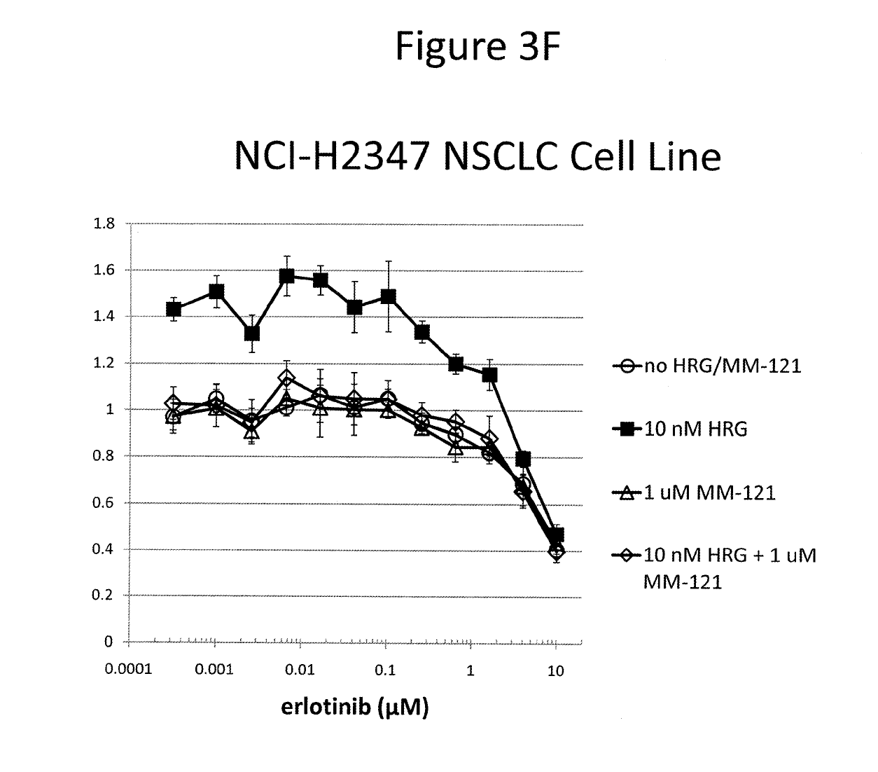

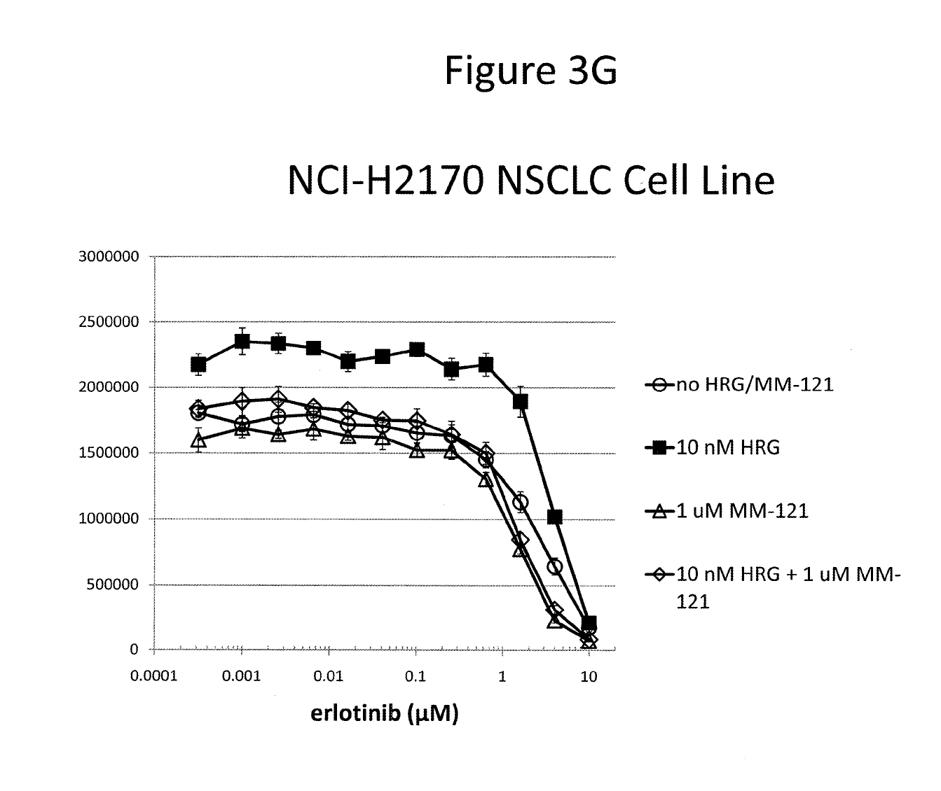

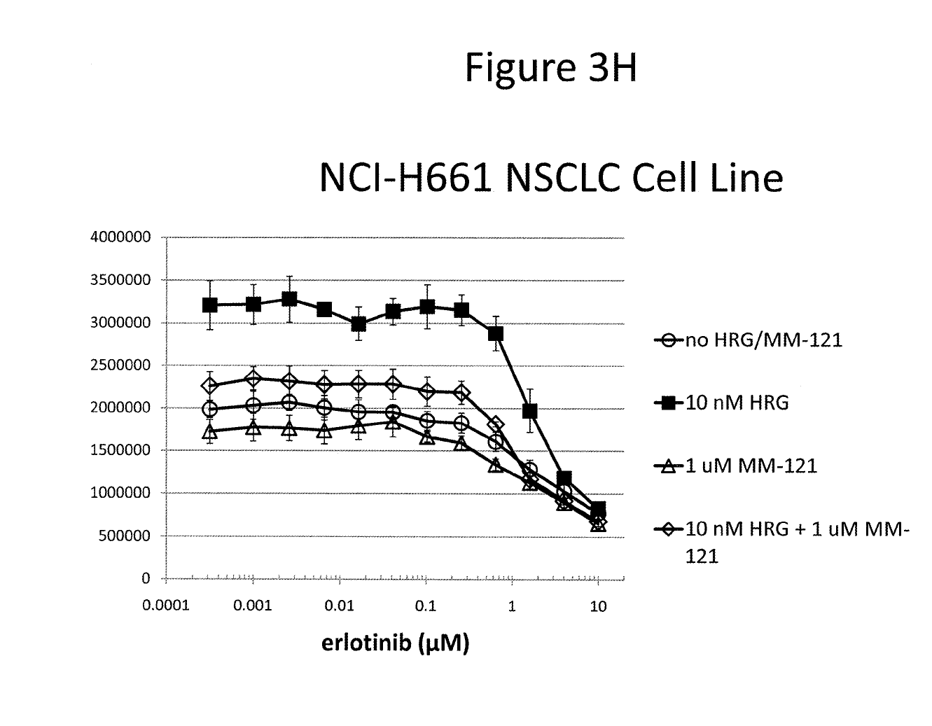

Eight cell lines were then each grown as spheroids as described above and treated with doses of erlotinib ranging from 0 to 10 μM, in either the absence or presence of heregulin-1β1 (HRG), and in either the absence or presence of MM 121. As shown in FIG. 3, while erlotinib was not able to inhibit cell growth in HRG-stimulated cells except at high concentrations; however, addition of MM-121 was able to restore sensitivity of the spheroids to erlotinib in the presence of HRG. This was true for all cell spheroids tested, including adenocarcinoma cell lines NCI-H322M (FIG. 3A); EKVX (FIG. 3B); A549 (FIG. 3C); NCI-H358 (FIG. 3D); NCI-H441 (FIG. 3E), NCI-H2347 (FIG. 3F); the squamous cell carcinoma cell line NCI-H2170 (FIG. 3G), and the large cell carcinoma cell line NCI-H661 (FIG. 3H). The mutation state of the K-Ras gene did not affect the effectiveness of MM-121 in increasing sensitivity of the cells to erlotinib.

These results demonstrate that HRG-mediated signaling may play a part in resistance to erlotinib in EGFR wild-type cells. The data further demonstrate that MM-121 is effective in restoring sensitivity of tumor cells to erlotinib in combination therapy.

### Example 3

### Inhibition of pAKT Production in Human Ovarian Cancer Cells In Vitro

### Materials and Methods:

The A2780 human ovarian cancer cell line was originally established from tumor tissue from an untreated patient. The A2780cis cell line is cisplatin-resistant (Cat. #93112517, Sigma). It was developed by chronic exposure of the parent cisplatin-sensitive A2780 cell line (Cat. #93112519, Sigma) to increasing concentration of cisplatin. A2780cis is cross-resistant to melphalan, adriamycin and irradiation. In order to maintain resistance, cisplatin is added to the culture media every 2-3 passages, post-attachment.

Resistance to cisplatin is confirmed by treating A2780 and A2780cis cells for 72 hours with a serial dilution of cisplatin (0.01 to 10 μM). Cell viability is measured using the Cell Titer Glo assay (Cat. # G7570, Promega) according to the manufacturer's instructions.

The effect of cisplatin on the AKT pathway is determined by treating sensitive (A2780) and resistant (A2780cis) cells with a dose range of cisplatin (0.1 to 10 μM) in vitro for 1, 4, 24 or 72 hours. After incubation cell lysates are prepared and analyzed for pAKT (ser473) (Cat #9271 Cell Signaling Technologies) by western blot.

The effect of MM-121 on the AKT pathway is determined by treating sensitive (A2780) and resistant (A2780cis) cells with a dose range of MM-121 ((0.01 to 1 μM) in vitro for 1, 4, 24 or 72 hours. After incubation cell lysates are prepared and analyzed for pAKT (ser473) (Cat. #9271 Cell Signaling Technologies) by western blot.

### Results:

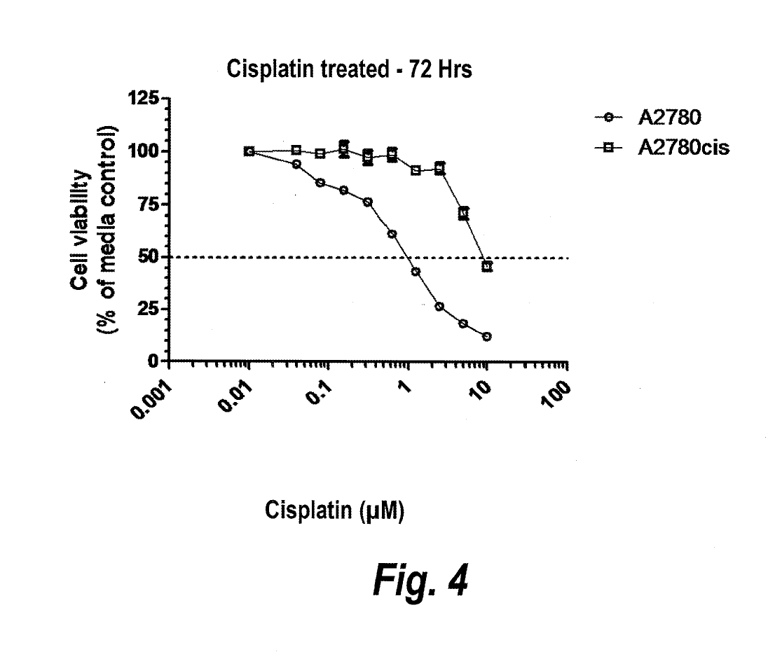

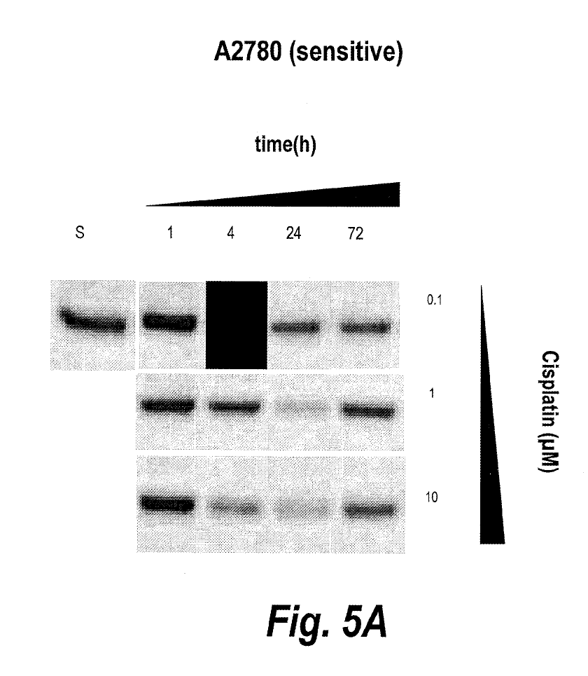

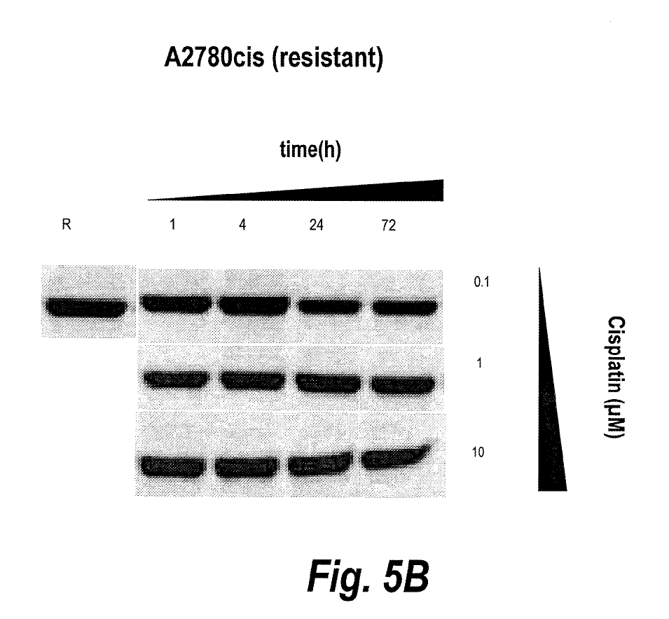

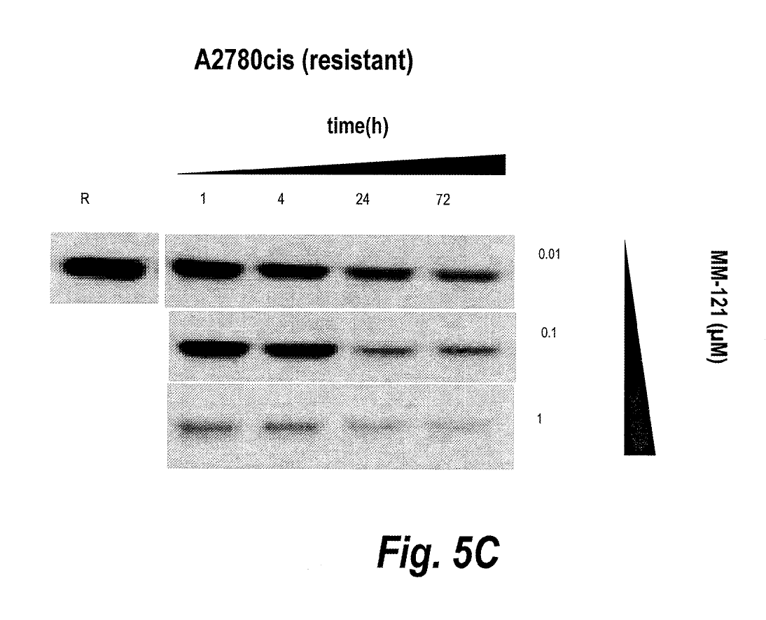

Resistance of the cell line A2780cis to treatment to cisplatin was evaluated by the methods described above or minor variations thereof. Resistant A2780cis and sensitive A2780 cells were plated and treated for 72 hours in vitro with a serial dilution of cisplatin (0.01 to 10 μM). Cell viability was measured by the Cell Titer Glo assay which measures metabolically active cells by quantitating ATP. As shown in FIG. 4, the A2780cis cells had a much greater viability (shown as percentage of media control) than did the sensitive line, A2780.

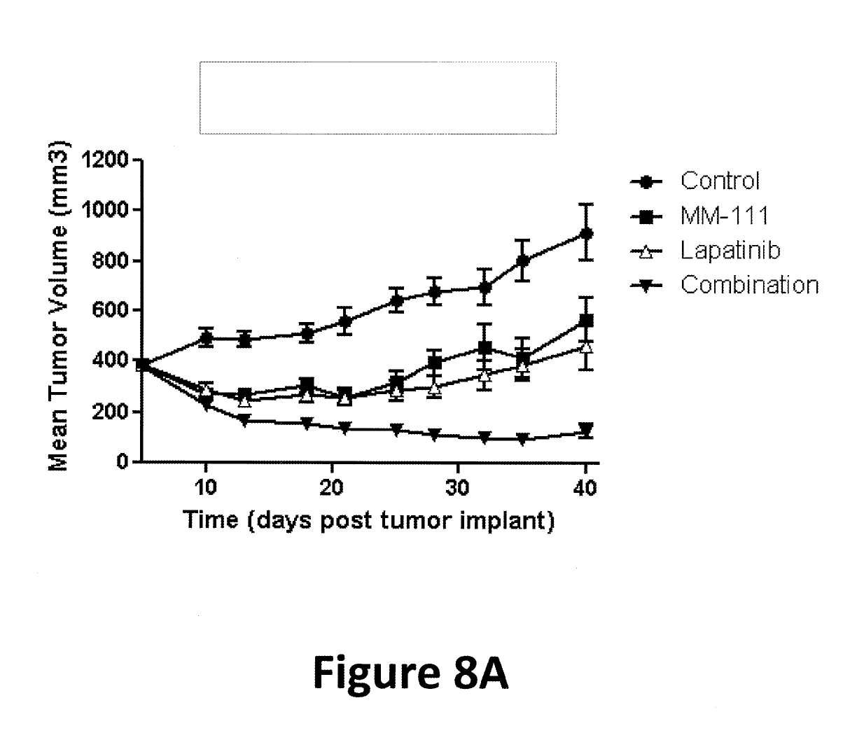

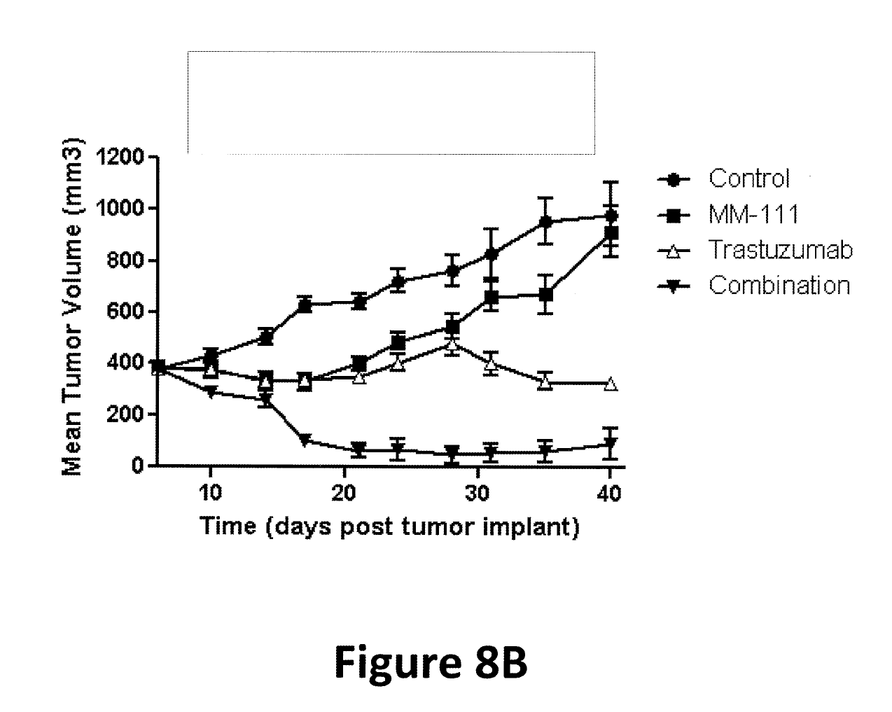

In order to characterize the effect of cisplatin on the AKT pathway in vitro, A2780 and A2780cis cells were treated with a dose range of cisplatin as described above for 1, 4 (A2780cis only), 24, or 72 hours. Cells sensitive to cisplatin showed a decrease in pAKT production (FIG. 8A) especially after 24 hours of treatment and/or at high cisplatin concentration (10 μM), whereas the cisplatin resistant cells showed no effect on pAKT production (FIG. 8B). As shown in FIG. 8C, these cells did show a reduction in AKT phosphorylation after treatment with MM-121. A2780cis cells were treated with a dose range of MM-121 as described above for 1, 4, 24, or 72 hours. Cells showed a gradual decrease in pAKT production at all dose levels and increasing over time.

### Example 4

### MM-121 Rescues Cisplatin Resistance in a Human Ovarian Cancer Cell Line in Vitro

In order to determine whether MM-121 can rescue the cisplatin-resistant phenotype of A2780cis cells in vitro, resistant A2780cis and sensitive A2780 cells will be plated as described above and treated with a dose range of cisplatin, MM-121, or a combination thereof for 1, 4, 24, or 72 hours. Cell lysates are analyzed by western blot for pAKT. Cells sensitive to cisplatin will show a reduction in pAKT levels after treatment with cisplatin and MM-121, and an even greater decrease in pAKT for the cells treated with both cisplatin and MM-121. Cells resistant to cisplatin will show no reduction in pAKT after treatment with cisplatin, and a moderate amount of reduction of pAKT after treatment with MM-121. A2780cis cells treated with a combination of MM-121 and cisplatin will show a greater decrease in the amount of pAKT after treatment than cells treated with MM-121 alone, suggesting an additive effect on the inhibition PI3K/AKT signaling and a restoration of sensitivity to cisplatin.

### Part II: Use of Bispecific Anti-ErbB3, Anti-ErbB2 Antibodies for Overcoming Resistance to ErbB Pathway Inhibitors

### Methods

### In Vitro Breast Cancer Model

BT474-M3 cells (see Noble, Cancer Chemother. Pharmacol. 2009 64:741-51) are treated with dose ranges of lapatinib, trastuzumab or MM-111 in the presence or absence of 5 nM heregulin. Viable cells are counted following 6 days of treatment. The effect of MM-111 combined with lapatinib or trastuzumab on inhibition of AKT phosphorylation is assessed in heregulin-stimulated BT474-M3 cells across a dose range.

### In Vivo Breast Cancer Xenograft Model

BT474-M3 cells (2×10⁷ cells per mice) are inoculated subcutaneously into Nu/Nu immunodeficient mice, which are implanted with an estrogen pellet (0.72 mg; 60-day release) one day before the experiment. Tumors are measured after seven days and mice are randomized into four groups: those treated with placebo, MM-111 (66 mg/kg, q7d), lapatinib (150 mg/kg q1d), or a combination of MM-111 and lapatinib. Tumors are measured twice a week and the experiment is ended at day 40. BT474-M3 breast tumor xenograft models are also treated with MM-111 (3 mg/kg q3d), trastuzumab (1 mg/kg q7d) or a combination of both drugs at these doses.

### Development of Trastuzumab-Resistance Cell Line

To establish trastuzumab resistant cells, BT474-M3 cells are cultured in RPMI1640 medium with 100 nM trastuzumab for six months, and 200 nM trastuzumab for two months, and then the dose level is increased to 500 nM. Cells are assayed in cell proliferation periodically to check the resistance level to trastuzumab.

### Cell Proliferation Assay

BT474-M3 parental (wild-type) and trastuzumab-resistant cells are plated in 96-well plates (3000 cells/well). After overnight incubation, cells are treated with a series dilution of trastuzumab or MM-111. After five days of treatment, cell viability is measured by WST-1 (Roche, Cat. #5015944001) according to manufacturer's instructions. Cells treated with control (RPMI1640 with 10% FBS) are set as 100%, other treatments are expressed as percentage of the control.

### Flow Cytometry Analysis

To determine the receptor status on BT474-M3 parental and trastuzumab-resistant cells, cells are trypsinized and washed with FACS buffer. Cells are then incubated with Alexa Fluor® 647-labeled mouse anti-ErbB2 antibody (BioLegend® Cat. #324412), cetuximab antibody (anti-EGFR), and B12 antibody (anti-ErbB3) for 1 hour at 4° C. Following washing with FACS buffer, cells were analyzed by FACSCalibur™ (BD bioscience).

### Spheroid Assay

BT474-M3 wild type and trastuzumab-resistant cells (2000 cells/well) are plated in Ultra Low Cluster 96-well plates (Costar®, Corning, N.Y.). After overnight incubation, a series dilution of trastuzumab or MM-111 is introduced to the plate. Cells are cultured for six days. Spheroids are then examined by Nikon microscope and analyzed by MetaMorph® Image Analysis Software (Molecular Devices). The spheroid size from cells cultured in medium containing 10% FBS is set as a control.

### Example 5

### The Activities of Her2-Directed Agents Trastuzumab and Lapatinib are Attenuated by Heregulin Stimulated ErbB3 Signaling, while MM-111 Remains Active

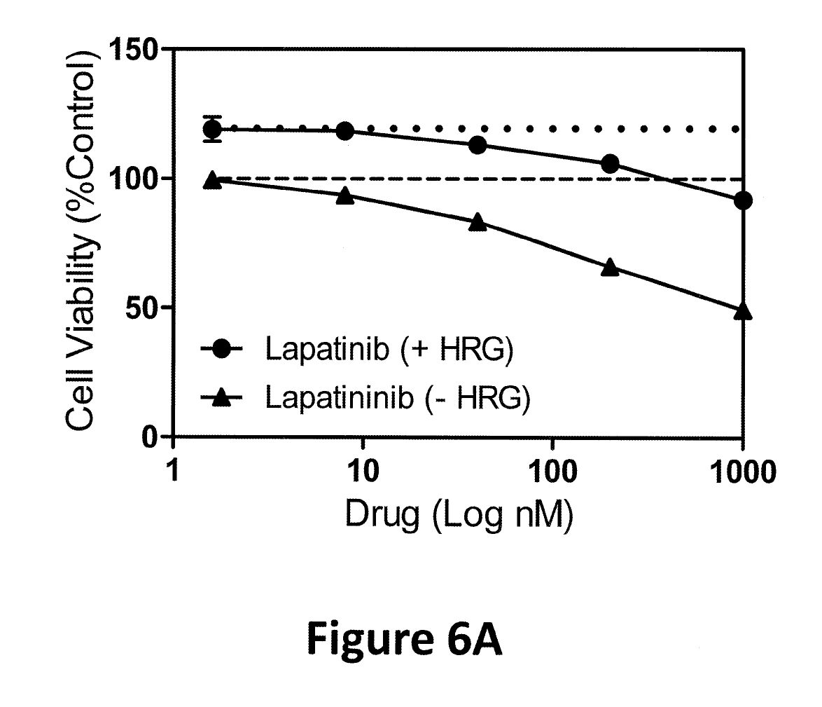

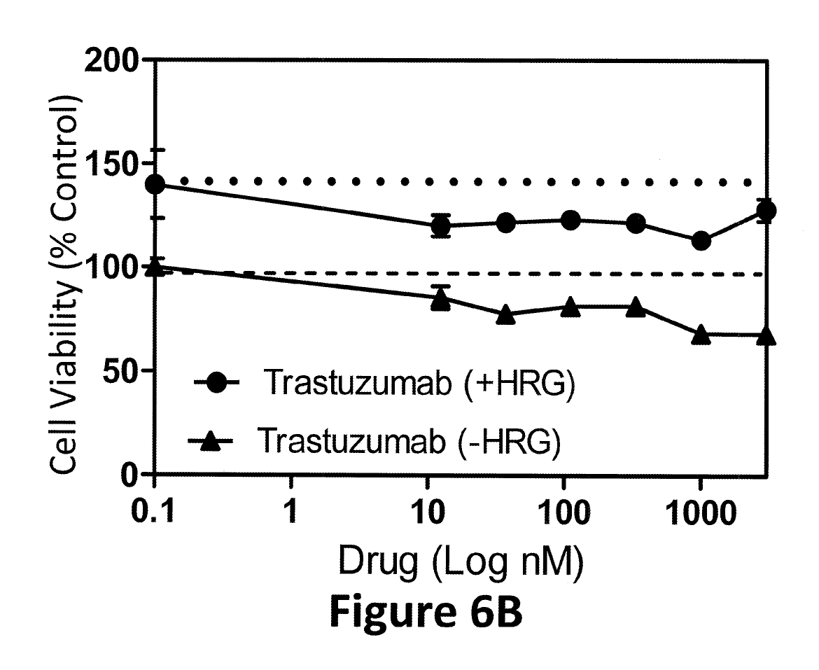

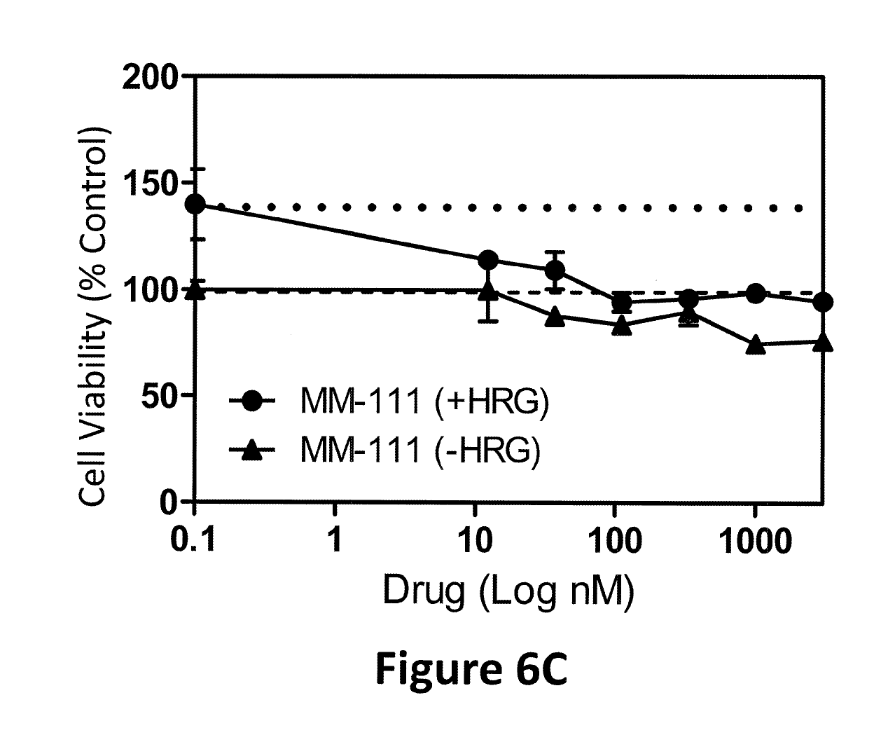

The ability of MM-111, lapatinib and trastuzumab to inhibit cell proliferation in the presence of heregulin was tested. Under basal conditions it was found that lapatinib (FIG. 6A), trastuzumab (FIG. 6B) and MM-111(FIG. 6C) inhibited BT474-M3 cell proliferation by 50%, 32% and 24%, respectively. When cells were cultured in the presence of 5 nM heregulin the effect of both lapatinib and trastuzumab was decreased so that inhibition of cell proliferation was reduced to 23% and 9%, respectively. Conversely, the inhibition of tumor cell growth by MM-111 was retained when heregulin was present, with 33% growth inhibition observed.

### Example 6

### The Addition of MM-111 to Lapatinib or Trastuzumab Increases pAKT Inhibition

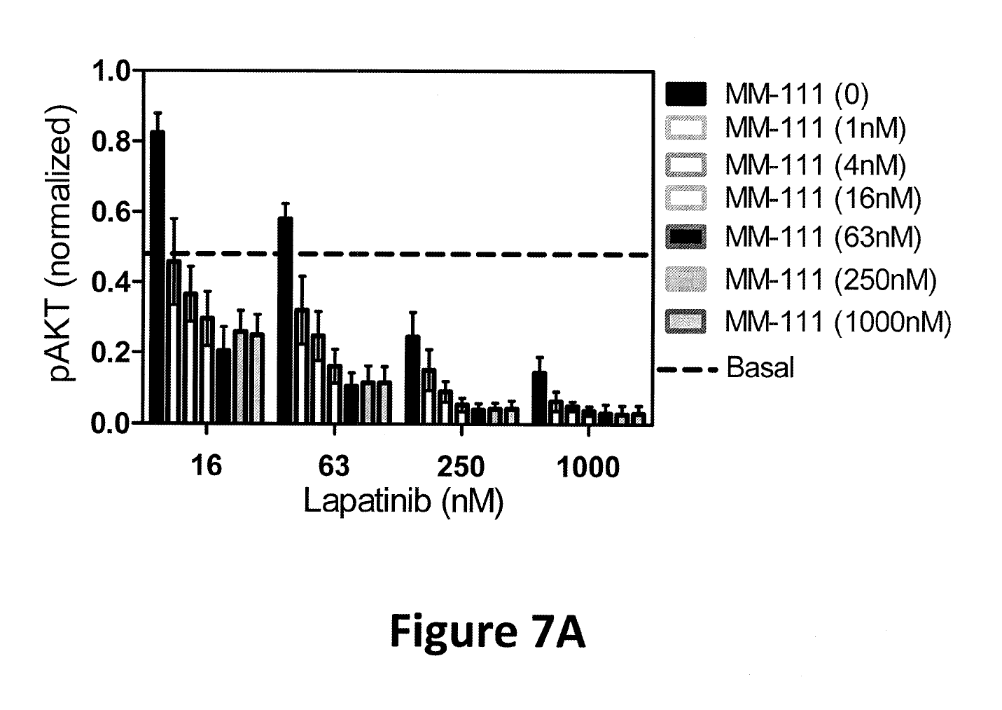

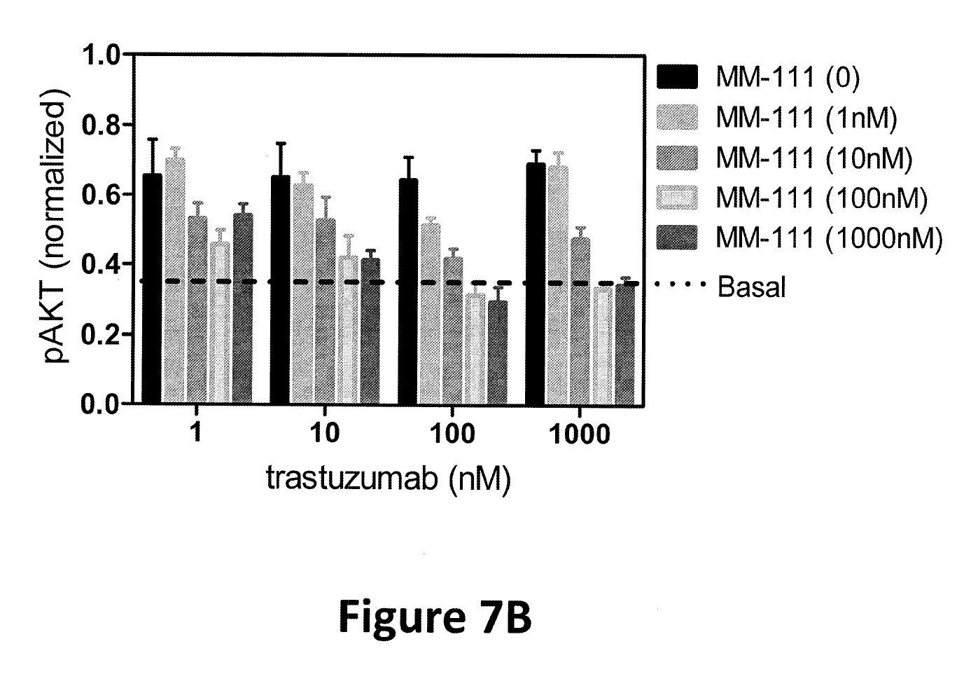

The ability of the combination of MM-111 and lapatinib or MM-111 and trastuzumab to inhibit AKT phosphorylation (activation) in vivo was investigated. While it was found that lapatinib alone inhibited phosphorylation of AKT in the presence of heregulin, the combination of MM-111 and lapatinib was extremely effective, inhibiting phosphorylation of AKT well below basal levels at therapeutically relevant concentrations (FIG. 7A). Trastuzumab did not inhibit AKT phosphorylation following heregulin stimulation (FIG. 7B). However, the addition of MM-111 to trastuzumab improved the inhibition of heregulin-stimulated AKT phosphorylation that was observed for trastuzumab alone, with inhibition of pAKT almost to basal levels, suggesting an additive effect of the combination (FIG. 7B).

### Example 7

### The Addition of MM-111 to Lapatinib or Trastuzumab Potentiates In Vivo Activity

The combination of MM-111 with trastuzumab or lapatinib was investigated in vivo using the BT474-M3 breast cancer xenograft model. Sub-optimal monotherapy doses of MM-111 (3 mg/kg dosed every 3 days) and trastuzumab (1 mg/kg dosed weekly), were selected for combination experiments to allow observation of any differences in activity between monotherapy and combination groups. MM-111 administered at 3 mg/kg every 3 days provided similar exposure to a weekly dose of 1 mg/kg trastuzumab due to the different pharmacokinetic properties of each agent in mice. Tumor growth inhibition in groups dosed with the combination of 3 mg/kg MM-111 and 1 mg/kg trastuzumab was more potent, and reached statistical significance, compared to MM-111 alone and trastuzumab alone. Additionally, an increase in the number of completely regressed tumors was observed with combination treatment compared to the monotherapy treatment groups, in which no complete regressions were observed (FIG. 8A). MM-111 and lapatinib were each dosed at an optimal efficacious dose weekly and every day, respectively. The combination of MM-111 and lapatinib provided more potency compared to either drug alone (FIG. 8B).

### Example 8

### MM-111 is Active in a Trastuzumab-Resistant Cell Line

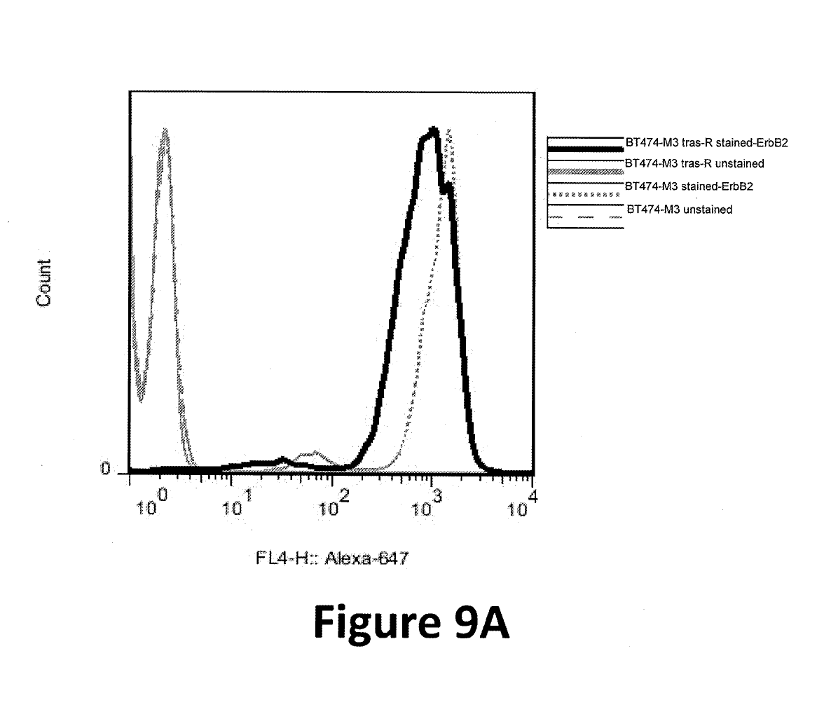

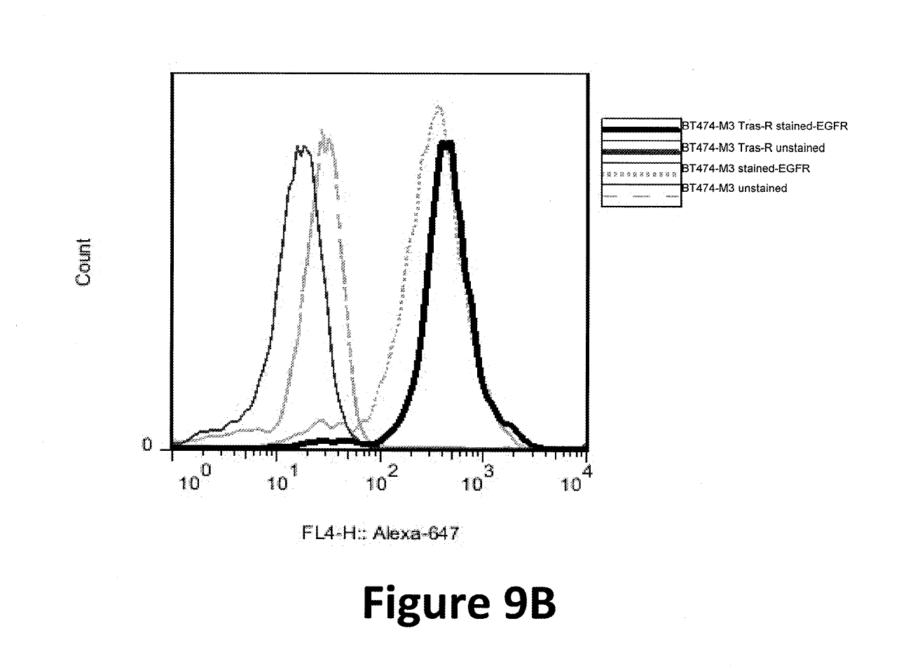

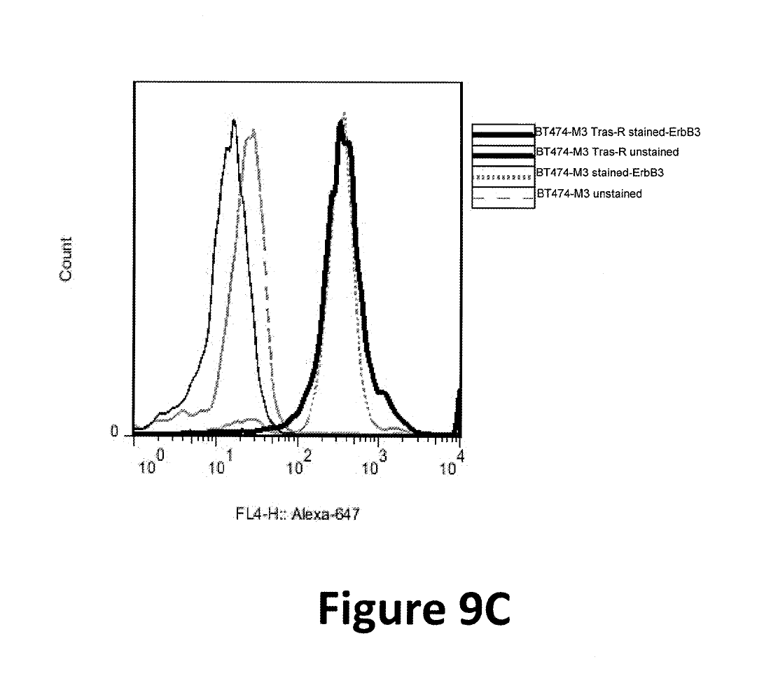

Flow cytometry analysis was performed to determine receptor status in wild-type and trastuzumab-resistant BT474-M3 cell lines. Wild-type or trastuzumab-resistant cells were stained with Alexa Fluor® 647-labeled mouse anti-ErbB2 antibody, cetuximab antibody (anti-EGFR), or B12 antibody (anti-ErbB3). Trastuzumab-resistant cells had a slightly decreased ErbB2 level (FIG. 9A) while EGFR (FIG. 9B) and ErbB3 (FIG. 9C) were unchanged.

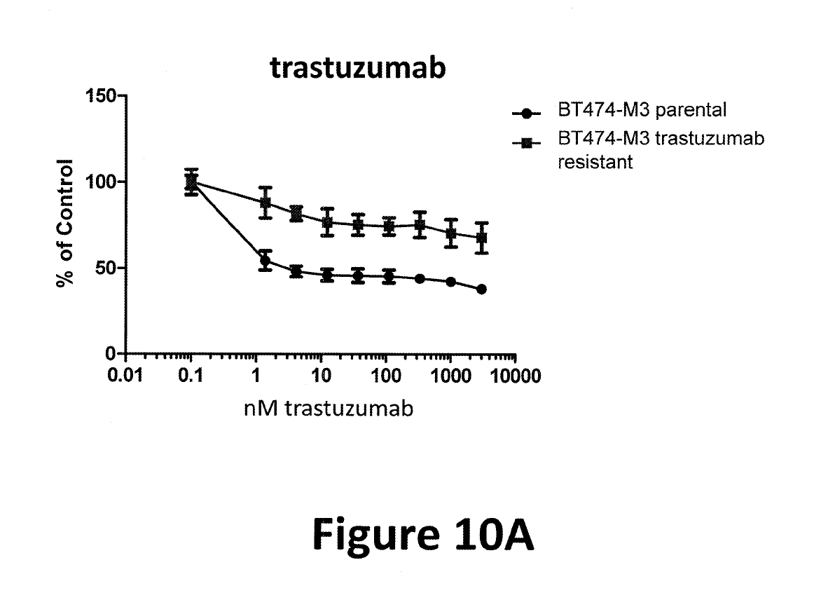

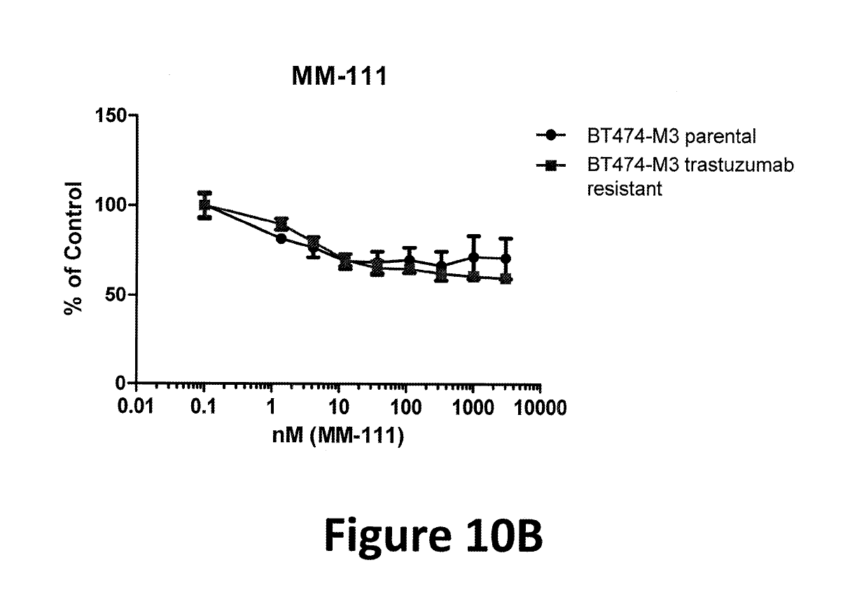

To determine the efficacy of MM-111 in inhibiting trastuzumab-resistant BT474-M3 cell proliferation, parental wild-type and trastuzumab-resistant BT474-M3 cells were treated as described above with a series dilution of either MM-111 or trastuzumab. While trastuzumab significantly inhibited cell proliferation in the parental cells, its inhibitory effect was significantly reduced in trastuzumab-resistant cells (FIG. 10A). In contrast, MM-111 maintained similar inhibitory activity in both parental and trastuzumab-resistant cells (FIG. 10B), thus demonstrating that MM-111 is able to circumvent the resistance mechanisms developed by cells after repeated exposure to trastuzumab.

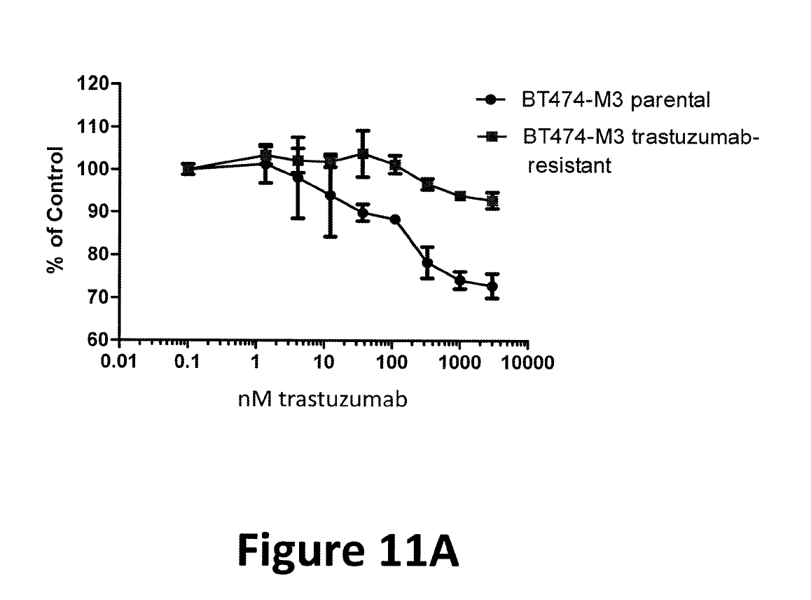

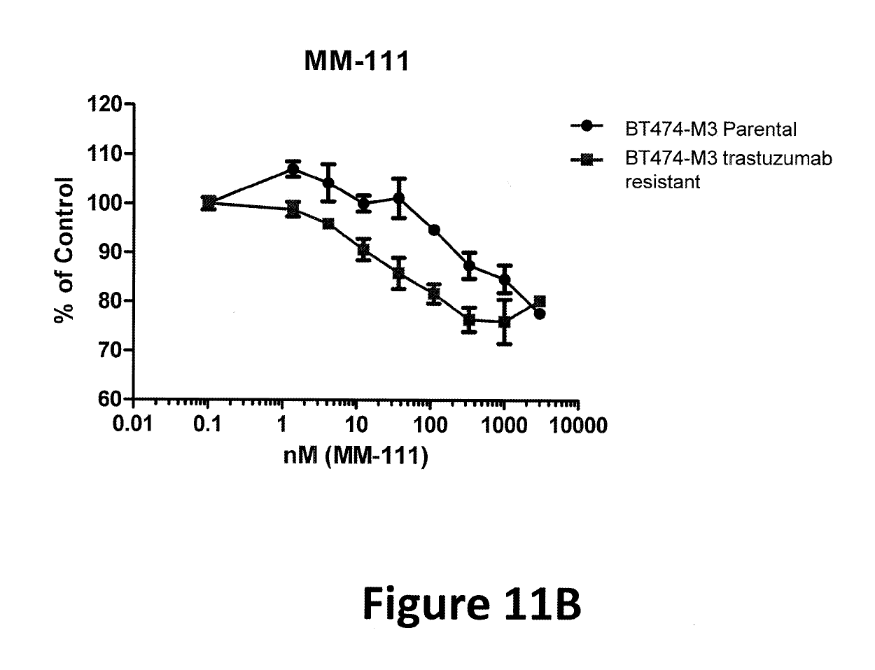

To further investigate the ability of MM-111 to inhibit cell growth in trastuzumab-resistant cells, multicellular spheroids of parental wild-type and trastuzumab-resistant BT474-M3 cells were prepared using the methods described above or minor variations thereof and treated with a series dilution of either MM-111 or trastuzumab. The inhibitory effect of trastuzumab was diminished in trastuzumab-resistant BT474-M3 cells, although it significantly inhibited spheroid growth of BT474-M3 parental cells (FIG. 11A). In contrast, MM-111 significantly reduced spheroid growth of both trastuzumab-resistant and wild-type BT474-M3 cells (FIG. 11B). Its inhibitory activity in trastuzumab-resistant cells was slightly improved when compared to its inhibitory activity in wild-type cells.

The data in the preceding Examples demonstrate that MM-111 is effective at inhibiting cell growth in cells that have developed resistance to trastuzumab.

### Example 9

### MM-111 but not Trastuzumab Combines with Anti-EGFR Therapeutics in Trastuzumab-Resistant BT474-M3 Cells

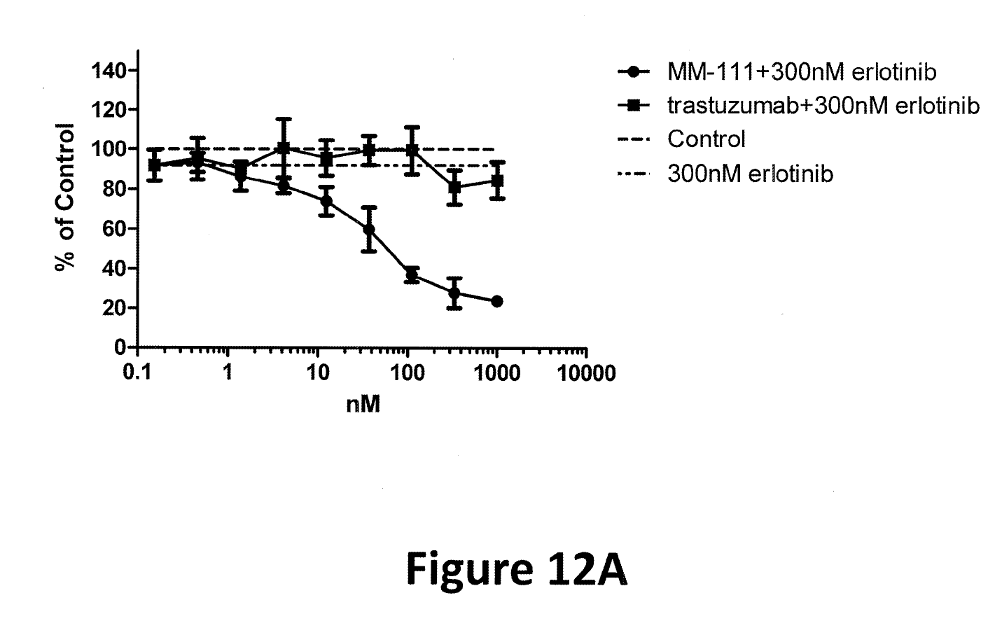

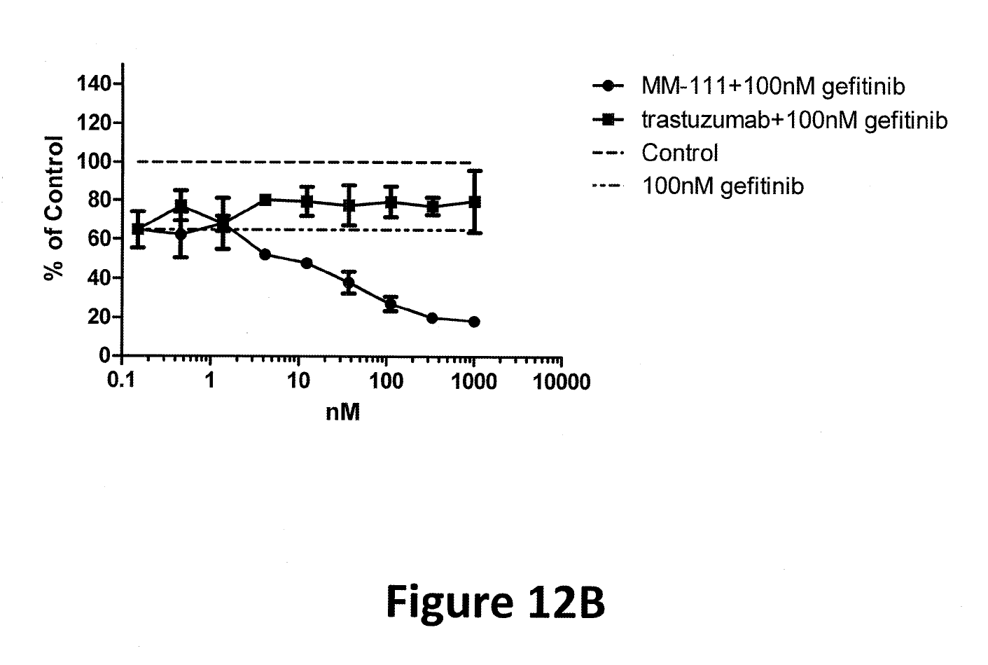

To compare the ability of MM-111 and trastuzumab to inhibit cell growth when in combination with EGFR inhibitors, spheroids of trastuzumab-resistant BT474-M3 cells were prepared using the methods described above and treated with a series of dilution of MM-111 and trastuzumab in the presence of either 300 nM erlotinib (FIG. 12A) or 100 nM gefitinib (FIG. 12B).”. As shown in FIGS. 12A and 12B, MM-111 but not trastuzumab was able to combine with erlotinib or gefitinib to reduce cell growth in trastuzumab-resistant cell spheroids. These data demonstrate that the combination MM-111 and an EGFR inhibitor is effective at inhibiting cell growth in cells that have developed resistance to trastuzumab. Furthermore, the combination of trastuzumab with an EGFR inhibitor was not sufficient to overcome the resistance of the cells.

### Example 10

### Combination Therapy with MM-111, Lapatinib and Tamoxifen in BT474-M3 Tumor Xenografts with and without Heregulin

### Methods

15×10⁶ BT474-M3 cells engineered to express GFP (BT474-M3-GFP) or BT474-M3 cells engineered to express GFP and heregulin 1 (BT474-M3-GFP-HRG) were implanted in to the mammary fat pads of estrogen supplemented (0.72 mg 17β-estradiol in a 60-day slow release biodegradable carrier) female NCr/NU-mice (Taconic Farms, Inc). When tumor volumes reached on average 516 mm³ (BT474-M3-GFP-HRG on day 17 after tumor implantation) or 422 mm³ (BT474-M3-GFP on day 20 after tumor implantation), mice were segregated into 8 groups of 10-15 mice. Groups received either no treatment (Control), MM-111 (48 mpk q3d i.p. (dose every three days, intraperitoneally)), lapatinib (150 mpk qd p.o. (oral dose daily)), tamoxifen (5 mg free base tamoxifen in a 60-day slow release biodegradable carrier), MM-111 and lapatinib, MM-111 and tamoxifen, lapatinib and tamoxifen or MM-111, lapatinib and tamoxifen. Tumors were measured twice a week with a digital caliber.

At the end of study (21 or 25 days after initiation of treatment for BT474-M3-GFP-HRG and BT474-M3-GFP, respectively) tumor samples were collected (24 h after the last MM-111 and 6 h after the last lapatinib dose) and analyzed for target and downstream signaling inhibition. Total and phosphorylated ErbB3 and Akt protein levels were analyzed from tumor lysates by suspension array technology (Luminex) and total and phosphorylated Erk1/2 levels by western blot using PCNA to normalize the results.

### Results

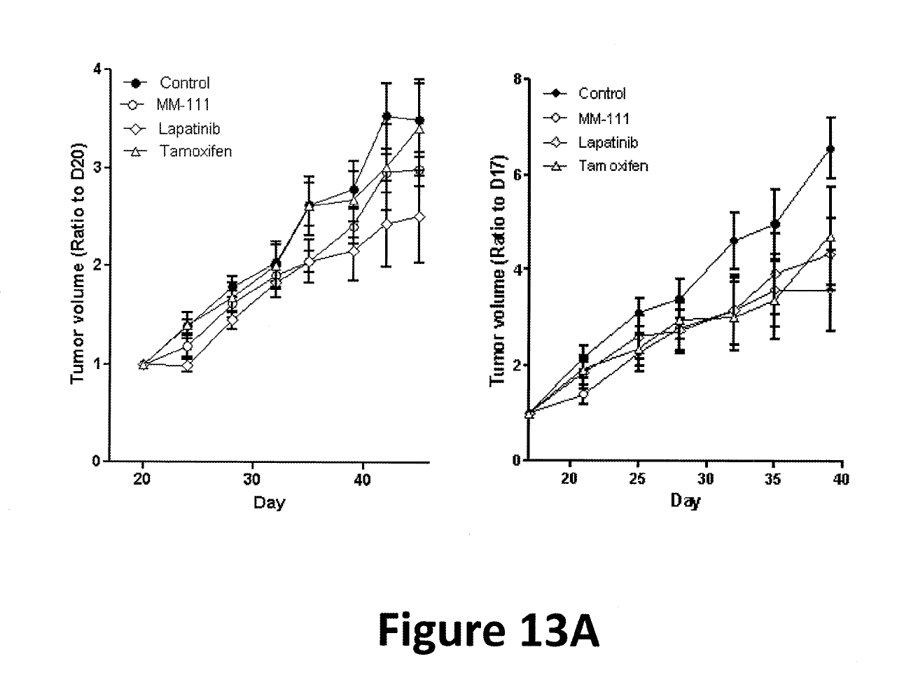

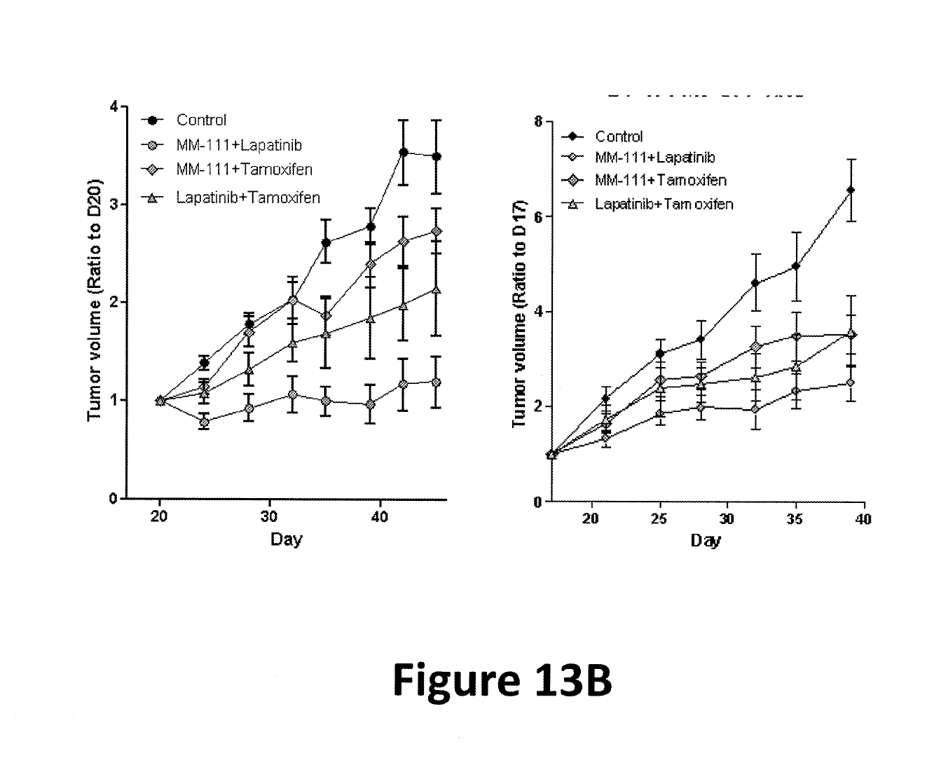

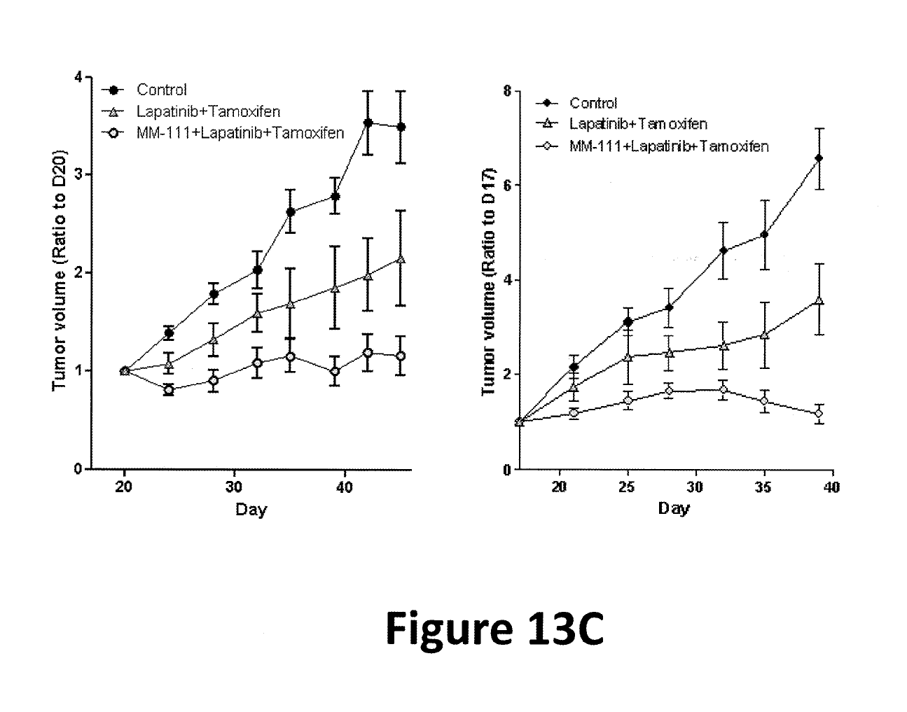

In order to demonstrate the effectiveness of MM-111 combination therapies to reduce tumor growth of heregulin (HRG) stimulated cells in vivo, combination therapies were tested in the BT474-M3-GFP and BT474-M3-GFP-HRG xenograft model according to the methods above. As shown in FIG. 13A, BT474-M3-GFP and BT474-M3-GFP-HRG tumor-bearing mice were treated with MM-111 (48 mpk), lapatinib (150 mpk) and tamoxifen (5 mg) monotherapies. Tumoral HRG overexpression improved tamoxifen efficacy and modestly improved MM-111 efficacy. BT474-M3-GFP and BT474-M3-GFP-HRG tumor bearing mice were then treated with MM-111 (48 mpk)+lapatinib (150 mpk), MM-111+tamoxifen (5 mg), and lapatinib+tamoxifen combination therapies. As shown in FIG. 13B, tumoral HRG overexpression modestly improved efficacy of the MM-111+tamoxifen combination. BT474-M3-GFP and BT474-M3-GFP-HRG tumor bearing mice were then treated with the combination of lapatinib+tamoxifen and MM-111+lapatinib+tamoxifen combination therapies. As shown in FIG. 13C, MM-111 greatly enhanced the efficacy of the lapatinib+tamoxifen combination therapy in both tumor models, demonstrating that MM-111 is required for the maximum anti-tumor efficacy regardless of the tumoral HRG expression.

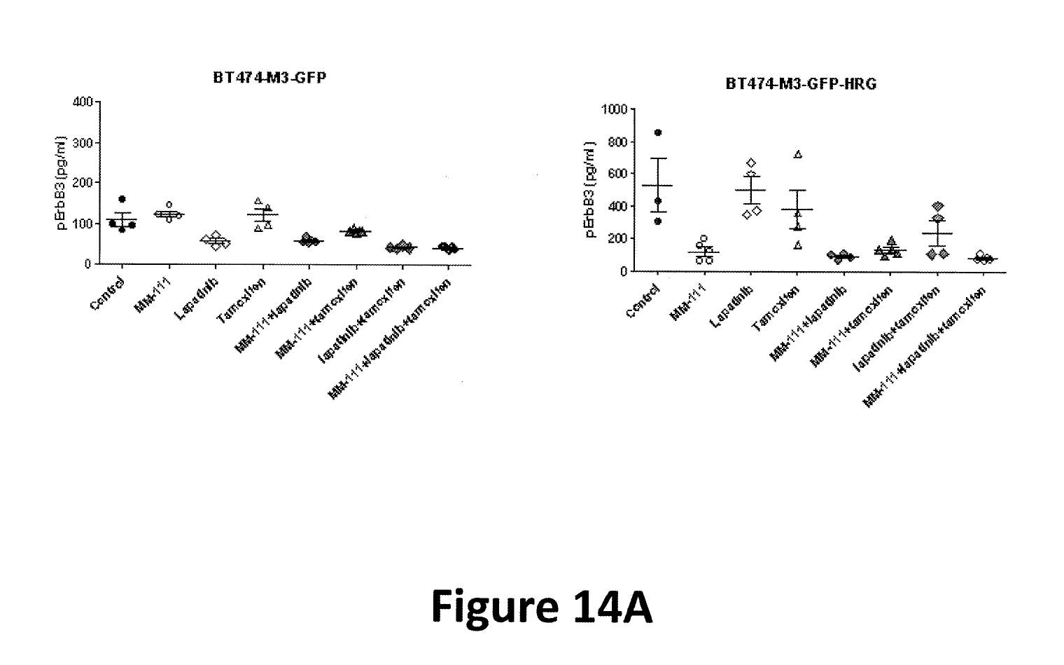

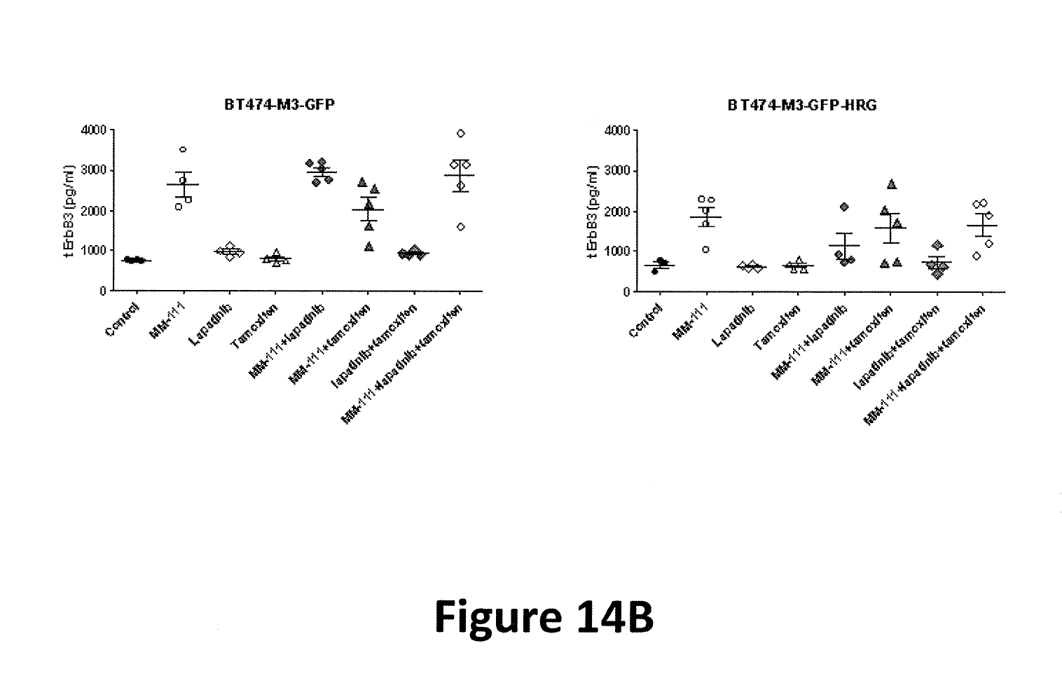

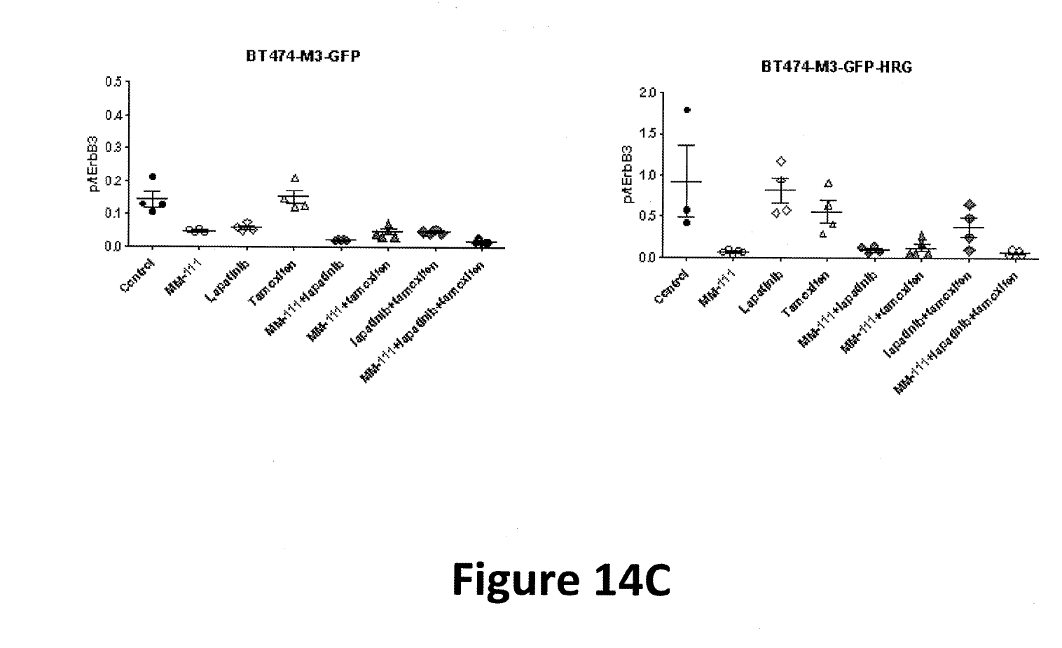

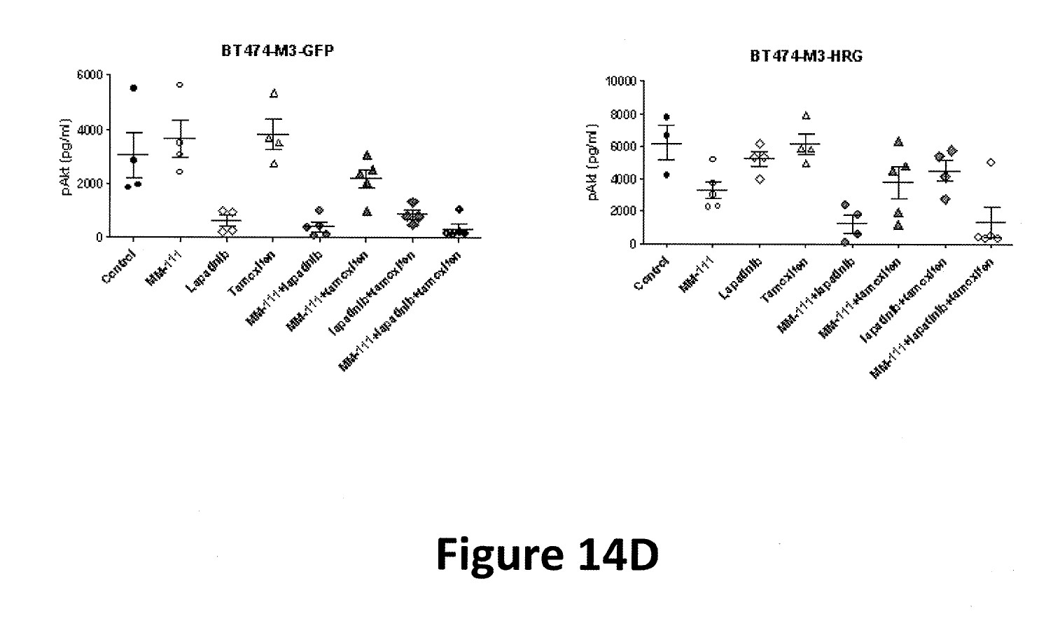

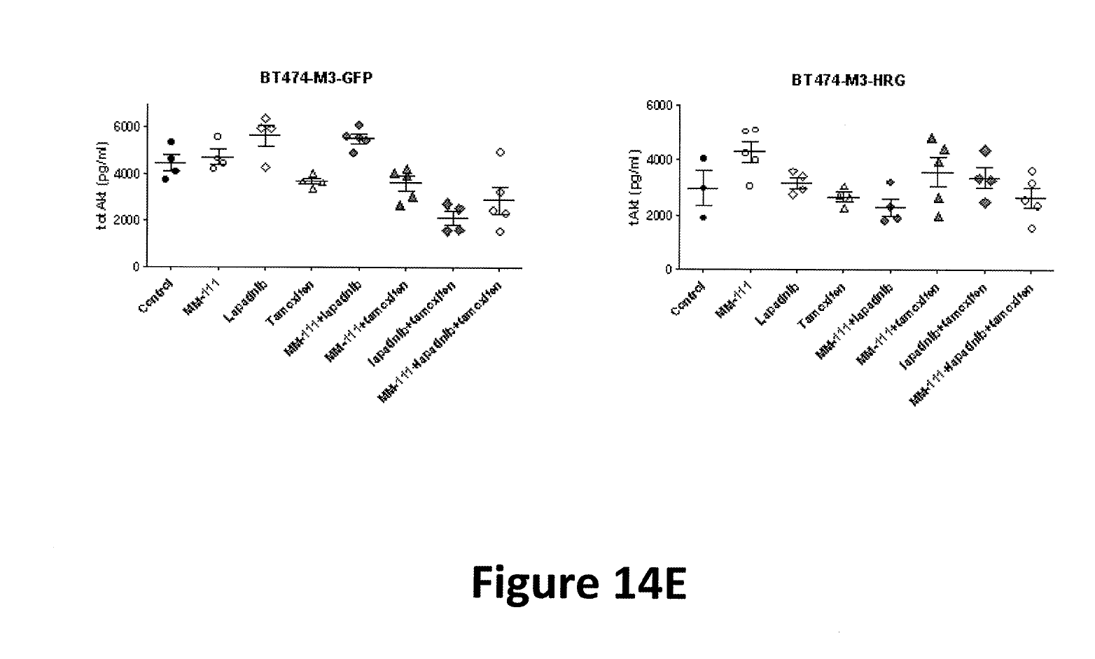

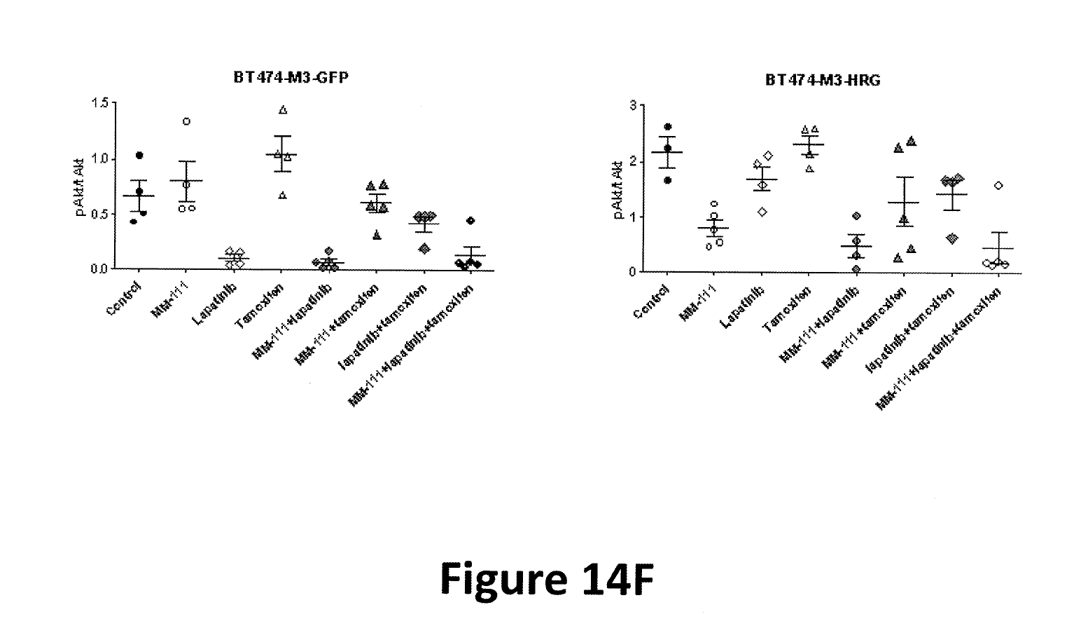

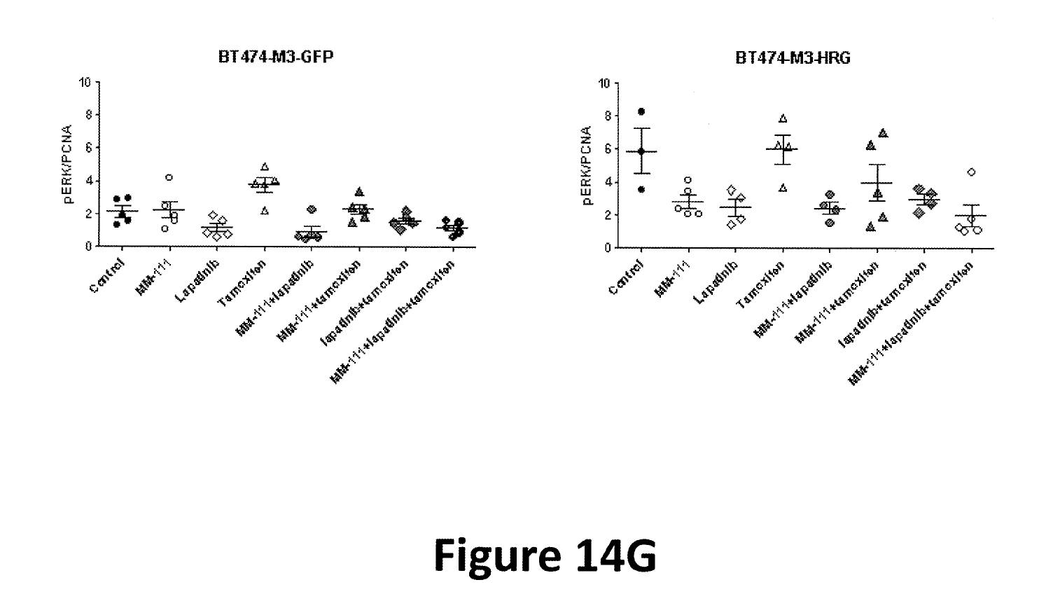

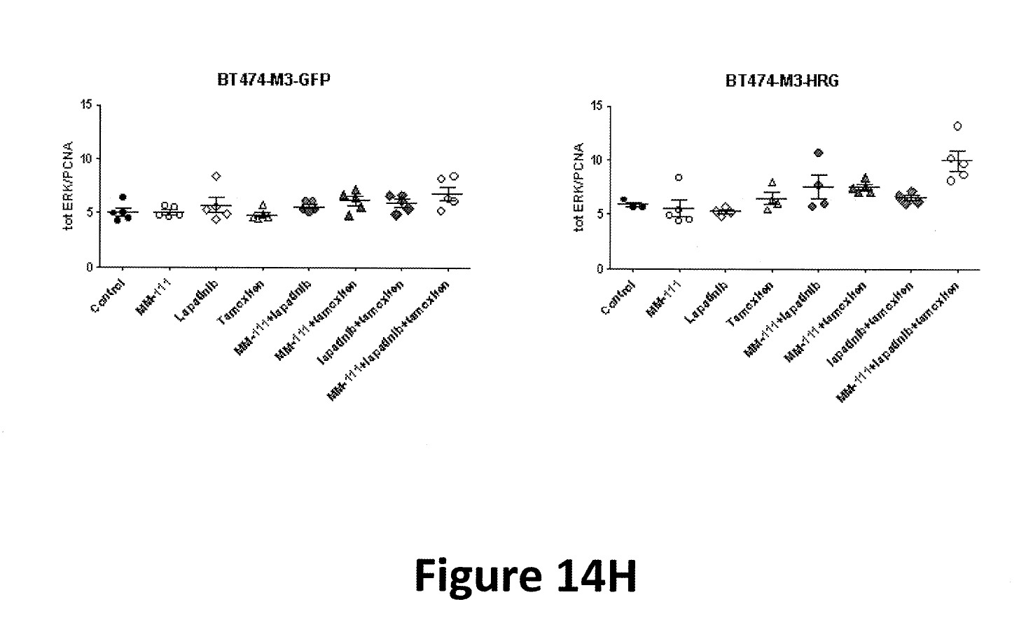

BT474-M3-GFP and BT474-M3-GFP-HRG tumor bearing mice (FIG. 14 left and right panels, respectively) were then treated with (from left to right) control (no treatment), MM-111, lapatinib, and tamoxifen monotherapies, the dual combinations of MM-111+lapatinib, MM-111+tamoxifen, and lapatinib+tamoxifen, and the triple combination. As shown in FIG. 14A which shows phospho-ErbB3 (pErbB3), tumoral HRG expression increased the ErbB3 phosphoprotein levels and lead to an increased effectiveness of MM-111 monotherapy and MM-111 combination therapy in reducing levels of pErbB3. Tumoral HRG expression lead to a decreased lapatinib and lapatinib+tamoxifen activity in reducing pErbB3 levels. As shown in FIG. 14B, MM-111 monotherapy and MM-111 combination therapies increased total ErbB3 expression in both tumor models. As shown in FIG. 14C, which shows the ratio of pErbB3 to total ErbB3 (tErbB3), both the MM-111 monotherapy and MM-111 combination therapy decreased ErbB3 activity even in the presence of HRG, whereas lapatinib and lapatinib+tamoxifen effectiveness was reduced in the presence of HRG.

As shown in FIG. 14F, which shows the ratio of phospho-Akt (pAkt, FIG. 14D) to total Akt (tAkt or totAkt, FIG. 14E), tumoral expression of HRG increased the Akt phosphoprotein/total protein levels and resulted in decreased effectiveness of the lapatinib and lapatinib+tamoxifen therapies, whereas the MM-111 monotherapy and combination therapies were effective at reducing pAkt production in the presence of HRG.

As shown in FIG. 14I, which shows the ratio of phospho-ERK (ERKt, FIG. 14G) to total Akt (totERK, FIG. 14H), tumoral expression of HRG lead to an increase in ERK1/2 phosphoprotein/total protein levels and resulted in decreased effectiveness of the lapatinib and lapatinib+tamoxifen therapies, whereas the MM-111 monotherapy and combination therapies were effective at reducing pERK production in the presence of HRG.

### Example 11

### MM-111 in Combination with the ToGA—Regimen in NCI-N87 Tumor Xenografts

The ToGA Study (Hoffmann-La Roche, ClinicalTrials.gov Identifier NCT01041404) was a study of trastuzumab in combination with chemotherapy compared with chemotherapy alone in patients with HER2-positive advanced gastric cancer. In that study, trastuzumab was administered as intravenous infusion of 6 mg/kg (loading dose 8 mg/kg) every 3 weeks. The chemotherapy consisted of a combination of 6 cycles of fluorouracil (800 mg/m²/day intravenous infusion every 3 weeks) and cisplatin (80 mg/m² intravenous infusion every 3 weeks), or capecitabine (1000 mg/m² p.o. twice daily for 14 days every 3 weeks) and cisplatin (80 mg/m² intravenous infusion every 3 weeks). Treatment with trastuzumab continued until disease progression. This treatment regimen was repeated in an NCI-N87 gastric cancer xenograft model according to the methods below.

### Methods

7.5×10⁶ NCI-N87 cells (ATCC® # CRL-5822™) were implanted subcutaneously in the flanks of female Nu/Nu (Charles River Laboratories, Inc.) mice. When tumor volumes reached on average 325 mm³ (on day 18 after tumor implantation) mice were segregated into 4 groups of 8-35 mice.

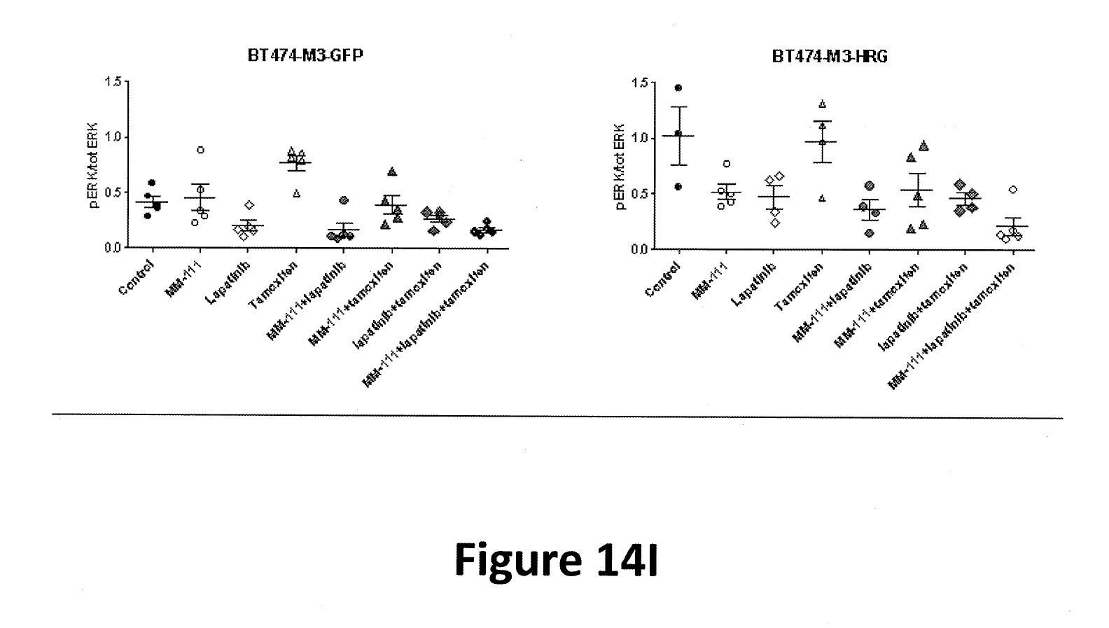

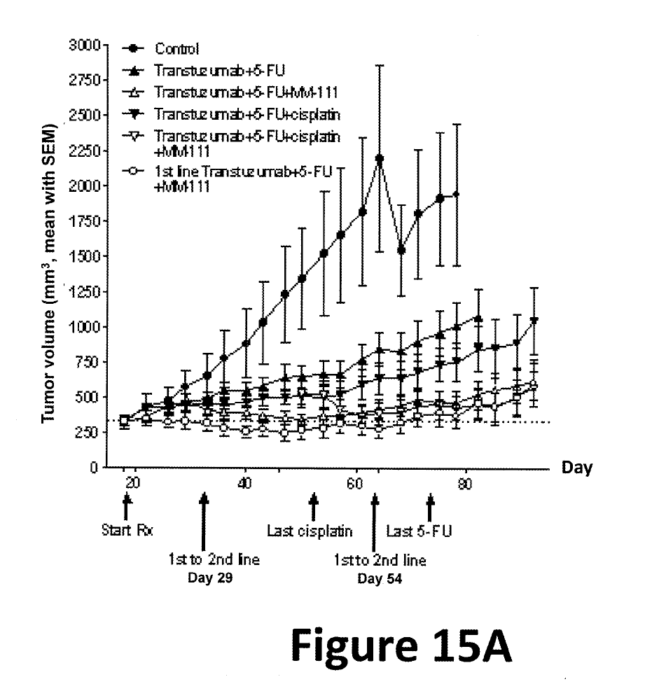

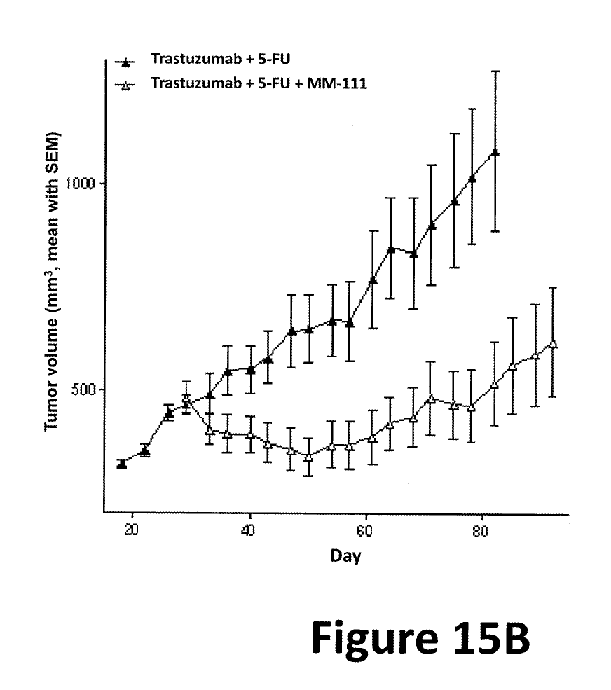

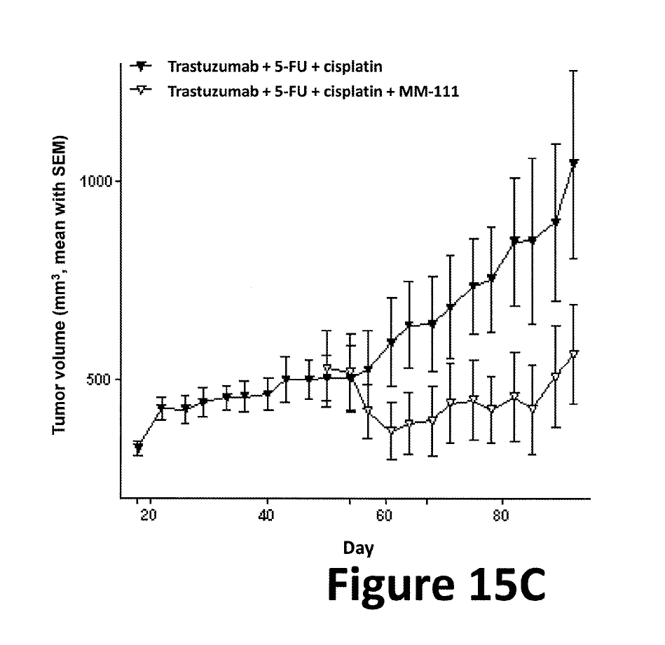

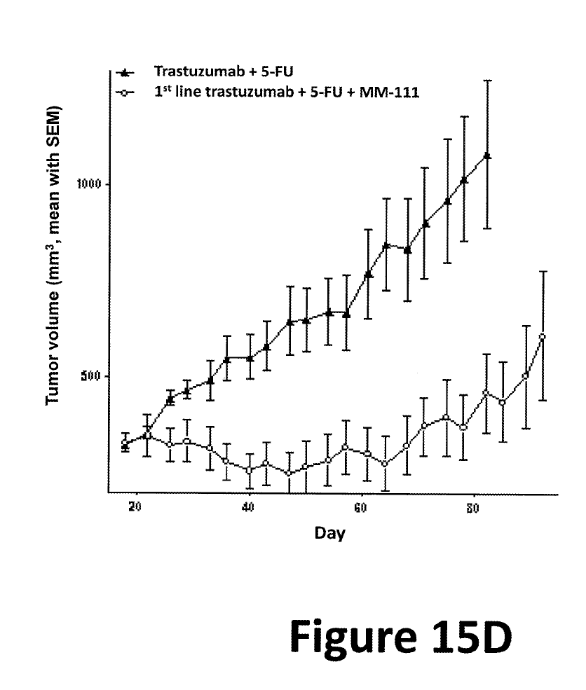

MM-111 was dosed either as a first line therapy at the initial treatment of the mice or as a second line therapy, wherein MM-111 was added to the treatment regimen (see arrows, FIG. 15A). Groups received either no treatment (Control), trastuzumab (3.5 mpk q3d i.p.)+5-FU (12 mpk qd, 5 times per week, i.p.), trastuzumab (3.5 mpk q3d i.p.)+5-FU (12 mpk qd, 5 times per week, i.p.)+cisplatin (5 mpk q7d i.p.) or 1^{st} line MM-111 (96 mpk q3d i.p.)+trastuzumab (3.5 mpk q3d i.p.)+5-FU (12 mpk qd, 5 times per week, i.p.). Tumors were measured twice a week with a digital caliber.

At the time of continued tumor growth on day 29, the trastuzumab+5-FU treatment group was divided into 2 treatment groups receiving either a) trastuzumab+5-FU or b) 2^{nd} line MM-111+trastuzumab+5-FU (see arrow, Day 29). Similarly, at the time of continued tumor growth on day 54, the trastuzumab+5-FU+cisplatin treatment group was divided into two treatment groups receiving either a) trastuzumab+5-FU+cisplatin or b) 2^{nd} line MM-111+trastuzumab+5-FU+cisplatin (see arrow, Day 54). Cisplatin administration had to be discontinued on day 52 and 5-FU administration had to be discontinued on day 64 due to animals showing signs of toxicities due to the chemotherapeutics. The discontinuation of the chemotherapies is indicated with arrows FIG. 15A.

### Results

FIG. 15A shows the tumor growth curves of NCI-N87 tumors treated as described above. FIGS. 15B-D each highlight a subset of the data shown in FIG. 15A. As shown in FIG. 15B, the addition of MM-111 as a second line therapy given to mice being treated with trastuzumab+5-FU resulted in an increased efficacy on tumor cell growth inhibition in tumors that had progressed on treatment with trastuzumab+5-FU alone. Similarly, as shown in FIG. 15C, the addition of MM-111 as a second line therapy given to mice being treated with trastuzumab+5-FU+cisplatin resulted in an increased efficacy on tumor cell growth inhibition in tumors that had progressed (resisted treatment) on treatment with trastuzumab+5-FU+cisplatin alone. Finally, as shown in FIG. 15D, treatment with MM-111 as a first line therapy in combination with trastuzumab and 5-FU prevented tumor growth for the first 60 days of the treatment, in contrast to treatment with the combination of trastuzumab+5-FU wherein the tumor volume increased from the beginning of the treatment.

### Example 12

### MM-111 in Combination with Trastuzumab and Paclitaxel in NCI-N87 Tumor Xenografts

### Methods

7.5×10⁶ NCI-N87 cells were implanted subcutaneously in the flanks of female Nu/Nu mice. When tumor volumes reached on average 341 mm³ (on day 24 after tumor implantation) mice were segregated into 4 groups of 10 mice. Groups received either no treatment (Control), paclitaxel (20 mpk q7d i.p.), trastuzumab (3.5 mpk q3d i.p.)+paclitaxel or MM-111 (48 mpk q3d i.p.)+trastuzumab+paclitaxel. Tumors were measured twice a week with a digital caliper.

### Results

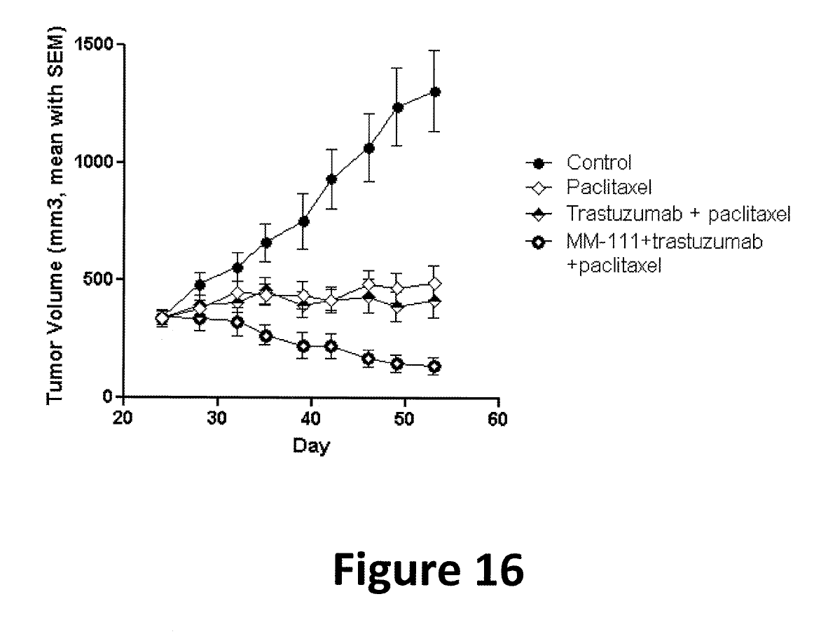

As shown in FIG. 16, the combination of MM-111 with trastuzumab+paclitaxel resulted in an increased efficacy on tumor cell growth inhibition in NCI-N87 tumors and resulted in continued tumor regression in contrast to paclitaxel alone and trastuzumab+paclitaxel, which caused only tumor stasis at best.

### Example 13

### MM-111/Lapatinib/Trastuzumab in BT474-M3 (±HRG) Tumor Xenografts

### Methods

15×10⁶ BT474-M3 cells engineered to express GFP (BT474-M3-GFP) or BT474-M3 cells engineered to express GFP and heregulin 1 (BT474-M3-GFP-HRG) were implanted in to the mammary fat pads of estrogen supplemented (0.72 mg 17β-estradiol in a 60-day slow release biodegradable carrier) female NCR/NU mice. When tumor volumes reached on average 286 mm³ (BT474-M3-GFP-HRG on day 14 after tumor implantation) or 305 mm³ (BT474-M3-GFP on day 16 after tumor implantation), mice were segregated into 8 groups of 8 mice. Groups received either no treatment (Control), MM-111 (48 mpk q3d i.p.), lapatinib (150 mpk qd p.o.), trastuzumab (3.5 mpk q3d i.p.), MM-111+lapatinib, MM-111+trastuzumab, lapatinib and trastuzumab or MM-111, lapatinib and trastuzumab. Tumors were measured twice a week with a digital caliber.

### Results

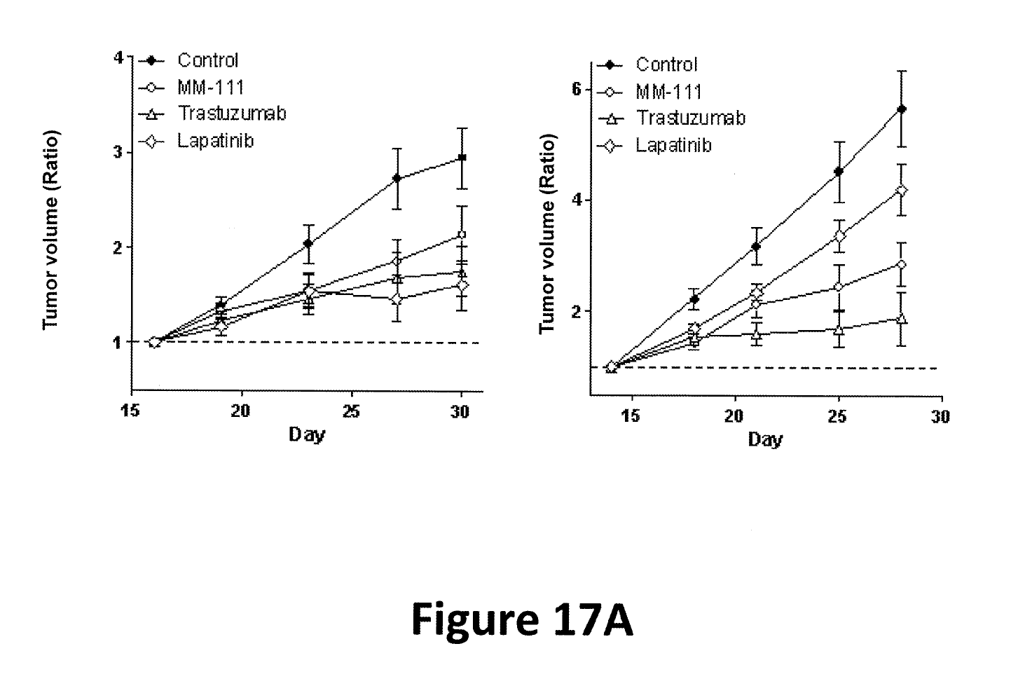

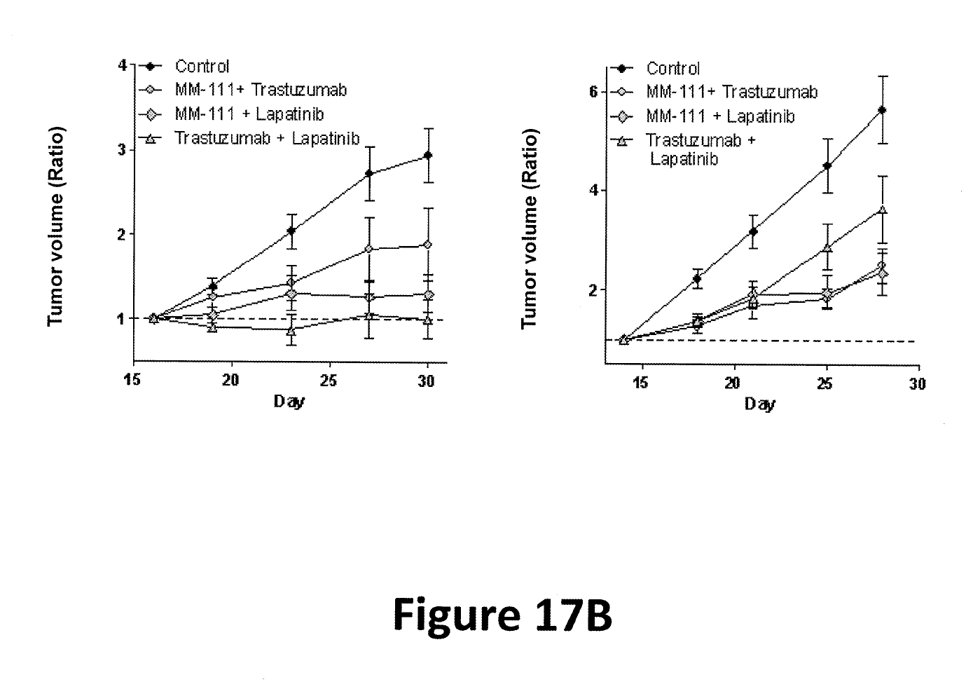

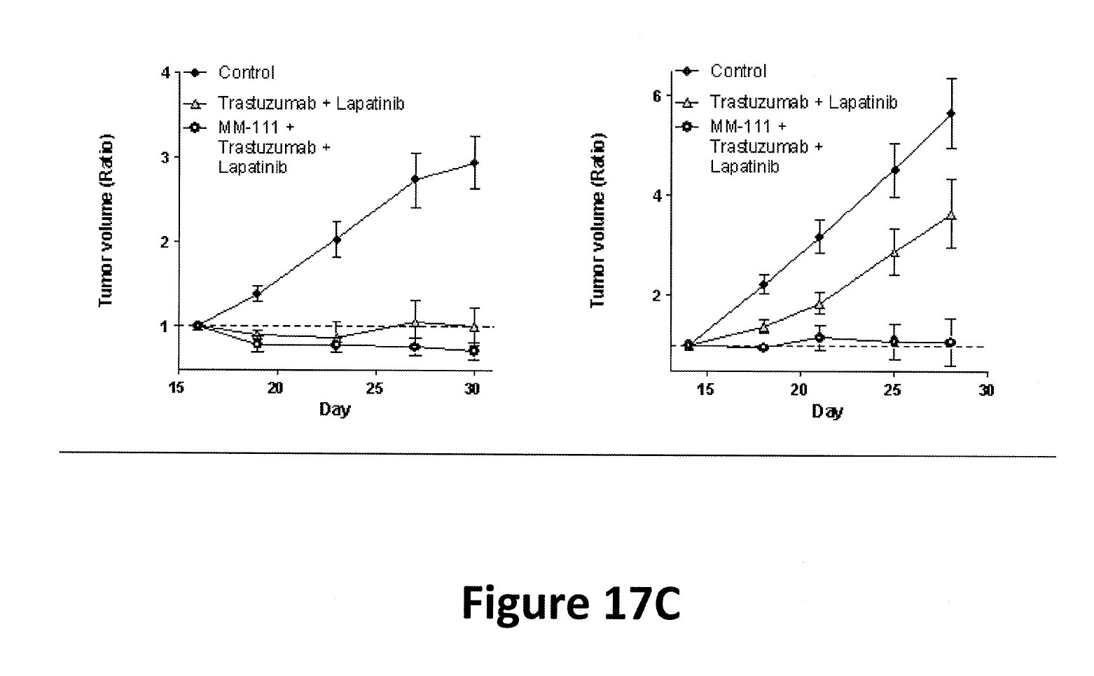

BT474-M3-GFP and BT474-M3-GFP-HRG tumor bearing mice were treated with MM-111, lapatinib and trastuzumab monotherapies (FIG. 17A), MM-111+lapatinib, MM-111+trastuzumab, and lapatinib+trastuzumab combination therapies (FIG. 17B), and lapatinib+trastuzumab and MM-111+lapatinib+trastuzumab combination therapies (FIG. 17C). As shown in FIG. 17B, tumoral HRG overexpression increased the efficacy of the MM-111+trastuzumab combination and decreased the efficacy of trastuzumab+lapatinib combination.

In addition, as shown in FIG. 17C, MM-111 greatly enhanced the efficacy of the lapatinib+trastuzumab combination therapy in the BT474-M3-GFP-HRG tumor model, demonstrating that MM-111 is required for the maximum anti-tumor efficacy when BT474-M3 tumors overexpress heregulin 1.

The results in the preceding Examples demonstrate the effectiveness of MM-111 in combination treatments, both as a 1^{st} line therapy to prevent development of resistance to other therapeutics and a 2^{nd} line therapy to re-sensitize tumor cells to treatment with other therapeutics.

### EQUIVALENTS

Those skilled in the art will recognize, or be able to ascertain using no more than routine experimentation, many equivalents of the specific embodiments of the invention(s) described herein. Such equivalents are intended to be encompassed by the following claims. Any combination of one or more of the embodiments disclosed in any independent claim and any of the dependent claims is also contemplated to be within the scope of the invention.

### INCORPORATION BY REFERENCE

Each and every patent, pending patent application, and publication referred to herein is hereby incorporated herein by reference in its entirety.

SUMMARY OF SEQUENCE LISTING

```
MM-121 (Ab # 6) V_{H} amino acid sequence (SEQ ID NO: 1)
EVQLLESGGGLVQPGGSLRLSCAASGFTFSHYVMAWVRQAPGKGLEWVSSISSSGGWT
LYADSVKGRFTISRDNSKNTLYLQMNSLRAEDTAVYYCTRGLKMATIFDYWGQGTLVT
VSS
MM-121 (Ab # 6) V_{L} amino acid sequence (SEQ ID NO: 2)
QSALTQPASVSGSPGQSITISCTGTSSDVGSYNVVSWYQQHPGKAPKLIIYEVSQRPSGVS
NRFSGSKSGNTASLTISGLQTEDEADYYCCSYAGSSIFVIFGGGTKVTVL
MM-121 (Ab # 6) V_{H} CDR1 (SEQ ID NO: 3)
HYVMA
MM-121 (Ab # 6) V_{H} CDR2 (SEQ ID NO: 4)
SISSSGGWTLYADSVKG
MM-121 (Ab # 6) V_{H} CDR3 (SEQ ID NO: 5)
GLKMATIFDY
MM-121 (Ab # 6) V_{L} CDR1 (SEQ ID NO: 6)
TGTSSDVGSYNVVS
MM-121 (Ab # 6) V_{L} CDR2 (SEQ ID NO: 7)
EVSQRPS
MM-121 (Ab # 6) V_{L} CDR3 (SEQ ID NO: 8)
CSYAGSSIFVI
MM-121 Heavy Chain Amino Acid Sequence (SEQ ID NO: 42)
    1 EVQLLESGGG LVQPGGSLRL SCAASGFTFS HYVMAWVRQA PGKGLEWVSS
  51 ISSSGGWTLY ADSVKGRFTI SRDNSKNTLY LQMNSLRAED TAVYYCTRGL
101 KMATIFDYWG QGTLVTVSSA STKGPSVFPL APCSRSTSES TAALGCLVKD
151 YFPEPVTVSW NSGALTSGVH TFPAVLQSSG LYSLSSVVTV PSSNFGTQTY
201 TCNVDHKPSN TKVDKTVERK CCVECPPCPA PPVAGPSVFL FPPKPKDTLM
251 ISRTPEVTCV VVDVSHEDPE VQFNWYVDGV EVHNAKTKPR EEQFNSTFRV
301 VSVLTVVHQD WLNGKEYKCK VSNKGLPAPI EKTISKTKGQ PREPQVYTLP
351 PSREEMTKNQ VSLTCLVKGF YPSDIAVEWE SNGQPENNYK TTPPMLDSDG
401 SFFLYSKLTV DKSRWQQGNV FSCSVMHEAL HNHYTQKSLS LSPGK
MM-121 Light Chain Amino Acid Sequence (SEQ ID NO: 43)
    1 QSALTQPASV SGSPGQSITI SCTGTSSDVG SYNVVSWYQQ HPGKAPKLII
  51 YEVSQRPSGV SNRFSGSKSG NTASLTISGL QTEDEADYYC CSYAGSSIFV
101 IFGGGTKVTV LGQPKAAPSV TLFPPSSEEL QANKATLVCL VSDFYPGAVT
151 VAWKADGSPV KVGVETTKPS KQSNNKYAAS SYLSLTPEQW KSHRSYSCRV
201 THEGSTVEKT VAPAECS
MM-121 (Ab # 6) Heavy Chain Nucleotide Sequence (SEQ ID NO: 45)
gaggtgcagc tgctggagag cggcggaggg ctggtccagc caggcggcag cctgaggctg
tcctgcgccg ccagcggctt caccttcagc cactacgtga tggcctgggt gcggcaggcc
ccaggcaagg gcctggaatg ggtgtccagc atcagcagca gcggcggctg gaccctgtac
gccgacagcg tgaagggcag gttcaccatc agcagggaca acagcaagaa caccctgtac
ctgcagatga acagcctgag ggccgaggac accgccgtgt actactgcac caggggcctg
aagatggcca ccatcttcga ctactggggc cagggcaccc tggtgaccgt gagcagc
MM-121 (Ab # 6) Light Chain Nucleotide Sequence (SEQ ID NO: 46)
cagtccgccc tgacccagcc cgccagcgtg agcggcagcc caggccagag catcaccatc
agctgcaccg gcaccagcag cgacgtgggc agctacaacg tggtgtcctg gtatcagcag
caccccggca aggcccccaa gctgatcatc tacgaggtgt cccagaggcc cagcggcgtg
agcaacaggt tcagcggcag caagagcggc aacaccgcca gcctgaccat cagcggcctg
cagaccgagg acgaggccga ctactactgc tgcagctacg ccggcagcag catcttcgtg
atcttcggcg gagggaccaa ggtgaccgtc cta
Ab # 3 V_{H} amino acid sequence (SEQ ID NO: 9)
EVQLLESGGGLVQPGGSLRLSCAASGFTFSAYNMRWVRQAPGKGLEWVSVIYPSGGAT
RYADSVKGRFTISRDNSKNTLYLQMNSLRAEDTAVYYCARGYYYYGMDVWGQGTLV
TVSS
Ab # 3 V_{L} amino acid sequence (SEQ ID NO: 10)
QSVLTQPPSASGTPGQRVTISCSGSDSNIGRNYIYWYQQFPGTAPKLLIYRNNQRPSGVP
DRISGSKSGTSASLAISGLRSEDEAEYHCGTWDDSLSGPVFGGGTKLTVL
Ab # 3 V_{H} CDR1 (SEQ ID NO: 11)
AYNMR
Ab # 3 V_{H} CDR2 (SEQ ID NO: 12)
VIYPSGGATRYADSVKG
Ab # 3 V_{H} CDR3 (SEQ ID NO: 13)
GYYYYGMDV
Ab # 3 V_{L} CDR1 (SEQ ID NO: 14)
SGSDSNIGRNYIY
Ab # 3 V_{L} CDR2 (SEQ ID NO: 15)
RNNQRPS
Ab # 3 V_{L} CDR3 (SEQ ID NO: 16)
GTWDDSLSGPV
Ab # 14 V_{H} amino acid sequence (SEQ ID NO: 17)
EVQLLESGGGLVQPGGSLRLSCAASGFTFSAYGMGWVRQAPGKGLEWVSYISPSGGHT
KYADSVKGRFTISRDNSKNTLYLQMNSLRAEDTAVYYCAKVLETGLLVDAFDIWGQGT
MVTVSS
Ab # 14 V_{L} amino acid sequence (SEQ ID NO: 18)
QYELTQPPSVSVYPGQTASITCSGDQLGSKFVSWYQQRPGQSPVLVMYKDKRRPSEIPE
RFSGSNSGNTATLTISGTQAIDEADYYCQAWDSSTYVFGTGTKVTVL
Ab # 14 V_{H} CDR1 (SEQ ID NO: 19)
AYGMG
Ab # 14 V_{H} CDR2 (SEQ ID NO: 20)
YISPSGGHTKYADSVKG
Ab # 14 V_{H} CDR3 (SEQ ID NO: 21)
VLETGLLVDAFDI
Ab # 14 V_{L} CDR1 (SEQ ID NO: 22)
SGDQLGSKFVS
Ab # 14 V_{L} CDR2 (SEQ ID NO: 23)
YKDKRRPS
Ab # 14 V_{L} CDR3 (SEQ ID NO: 24)
QAWDSSTYV
Ab # 17 V_{H} amino acid sequence (SEQ ID NO: 25)
EVQLLESGGGLVQPGGSLRLSCAASGFTFSWYGMGWVRQAPGKGLEWVSYISPSGGIT
VYADSVKGRFTISRDNSKNTLYLQMNSLRAEDTAVYYCARLNYYYGLDVWGQGTTVT
VSS
Ab # 17 V_{L} amino acid sequence (SEQ ID NO: 26)
QDIQMTQSPSSLSASVGDRITITCQASQDIGDSLNWYQQKPGKAPRLLIYDASNLETGVP
PRFSGSGSGTDFTFTFRSLQPEDIATYFCQQSANAPFTFGPGTKVDIK
Ab # 17 V_{H} CDR1 (SEQ ID NO: 27)
WYGMG
Ab # 17 V_{H} CDR2 (SEQ ID NO: 28)
YISPSGGITVYADSVKG
Ab # 17 V_{H} CDR3 (SEQ ID NO: 29)
LNYYYGLDV
Ab # 17 V_{L} CDR1 (SEQ ID NO: 30)
QASQDIGDSLN
Ab # 17 V_{L} CDR2 (SEQ ID NO: 31)
DASNLET
Ab # 17 V_{L} CDR3 (SEQ ID NO: 32)
QQSANAPFT
Ab # 19 V_{H} amino acid sequence (SEQ ID NO: 33)
EVQLLESGGGLVQPGGSLRLSCAASGFTFSRYGMWWVRQAPGKGLEWVSYIGSSGGPT
YYVDSVKGRFTISRDNSKNTLYLQMNSLRAEDTAVYYCAGGRGTPYYFDSWGQGTLV
TVSS
Ab # 19 V_{L} amino acid sequence (SEQ ID NO: 34)
QYELTQPASVSGSPGQSITISCTGTSSDIGRWNIVSWYQQHPGKAPKLMIYDVSNRPSGV
SNRF
SGSKSGNTASLTISGLQAEDEADYYCSSYTSSSTWVFGGGTKLTVL
Ab # 19 V_{H} CDR1 (SEQ ID NO: 35)
RYGMW
Ab # 19 V_{H} CDR2 (SEQ ID NO: 36)
YIGSSGGPTYYVDSVKG
Ab # 19 V_{H} CDR3 (SEQ ID NO: 37)
GRGTPYYFDS
Ab # 19 V_{L} CDR1 (SEQ ID NO: 38
TGTSSDIGRWNIVS
Ab # 19 V_{L} CDR2 (SEQ ID NO: 39)
DVSNRPS
Ab # 19 V_{L} CDR3 (SEQ ID NO: 40)
SSYTSSSTWV
ErbB3 (SEQ ID NO: 41)
SEVGNSQAVCPGTLNGLSVTGDAENQYQTLYKLYERCEVVMGNLEIVLTGHNADLSFL
QWIREVTGYVLVAMNEFSTLPLPNLRVVRGTQVYDGKFAIFVMLNYNTNSSHALRQLR
LTQLTEILSGGVYIEKNDKLCHMDTIDWRDIVRDRDAEIVVKDNGRSCPPCHEVCKGRC
WGPGSEDCQTLTKTICAPQCNGHCFGPNPNQCCHDECAGGCSGPQDTDCFACRHFNDS
GACVPRCPQPLVYNKLTFQLEPNPHTKYQYGGVCVASCPHNFVVDQTSCVRACPPDKM
EVDKNGLKMCEPCGGLCPKACEGTGSGSRFQTVDSSNIDGFVNCTKILGNLDFLITGLNG
DPWHKIPALDPEKLNVFRTVREITGYLNIQSWPPHMHNFSVFSNLTTIGGRSLYNRGFSLL
IMKNLNVTSLGFRSLKEISAGRIYISANRQLCYHHSLNWTKVLRGPTEERLDIKHNRPRRD
CVAEGKVCDPLCSSGGCWGPGPGQCLSCRNYSRGGVCVTHCNFLNGEPREFAHEAECFS
CHPECQPMEGTATCNGSGSDTCAQCAHFRDGPHCVSSCPHGVLGAKGPIYKYPDVQNEC
RPCHENCTQGCKGPELQDCLGQTLVLIGKTHLTMALTVIAGLVVIFMMLGGTFLYWRGR
RIQNKRAMRRYLERGESIEPLDPSEKANKVLARIFKETELRKLKVLGSGVFGTVHKGVWI
PEGESIKIPVCIKVIEDKSGRQSFQAVTDHMLAIGSLDHAHIVRLLGLCPGSSLQLVTQYLP
LGSLLDHVRQHRGALGPQLLLNWGVQIAKGMYYLEEHGMVHRNLAARNVLLKSPSQV
QVADFGVADLLPPDDKQLLYSEAKTPIKWMALESIHFGKYTHQSDVWSYGVTVWELMT
FGAEPYAGLRLAEVPDLLEKGERLAQPQICTIDVYMVMVKCWMIDENIRPTFKELANEFT
RMARDPPRYLVIKRESGPGIAPGPEPHGLTNKKLEEVELEPELDLDLDLEAEEDNLATTTL
GSALSLPVGTLNRPRGSQSLLSPSSGYMPMNQGNLGESCQESAVSGSSERCPRPVSLHPMP
RGCLASESSEGHVTGSEAELQEKVSMCRSRSRSRSPRPRGDSAYHSQRHSLLTPVTPLSPPG
LEEEDVNGYVMPDTHLKGTPSSREGTLSSVGLSSVLGTEEEDEDEEYEYMNRRRRHSPPHP
PRPSSLEELGYEYMDVGSDLSASLGSTQSCPLHPVPIMPTAGTTPDEDYEYMNRQRDGGGP
GGDYAAMGACPASEQGYEEMRAFQGPGHQAPHVHYARLKTLRSLEATDSAFDNPDYWH
SRLFPKANAQRT
MM-111 amino acid sequence (SEQ ID NO: 44)
QVQLQESGGGLVKPGGSLRLSCAASGFTFSSYWMSWVRQAPGKGLEWVANINRDGSA
SYYVDSVKGRFTISRDDAKNSLYLQMNSLRAEDTAVYYCARDRGVGYFDLWGRGTLV
TVSSASTGGGGSGGGGSGGGGSQSALTQPASVSGSPGQSITISCTGTSSDVGGYNFVSW
YQQHPGKAPKLMIYDVSDRPSGVSDRFSGSKSGNTASLIISGLQADDEADYYCSSYGSSS
THVIFGGGTKVTVLGAASDAHKSEVAHRFKDLGEENFKALVLIAFAQYLQQSPFEDHV
KLVNEVTEFAKTCVADESAENCDKSLHTLFGDKLCTVATLRETYGEMADCCAKQEPER
NECFLQHKDDNPNLPRLVRPEVDVMCTAFHDNEETFLKKYLYEIARRHPYFYAPELLFF
AKRYKAAFTECCQAADKAACLLPKLDELRDEGKASSAKQRLKCASLQKFGERAFKAW
AVARLSQRFPKAEFAEVSKLVTDLTKVHTECCHGDLLECADDRADLAKYICENQDSISS
KLKECCEKPLLEKSHCIAEVENDEMPADLPSLAADFVESKDVCKNYAEAKDVFLGMFL
YEYARRHPDYSVVLLLRLAKTYETTLEKCCAAADPHECYAKVFDEFKPLVEEPQNLIKQ
NCELFEQLGEYKFQNALLVRYTKKVPQVSTPTLVEVSRNLGKVGSKCCKHPEAKRMPC
AEDYLSVVLNQLCVLHEKTPVSDRVTKCCTESLVNRRPCFSALEVDETYVPKEFQAETF
TFHADICTLSEKERQIKKQTALVELVKHKPKATKEQLKAVMDDFAAFVEKCCKADDKE
TCFAEEGKKLVAASQAALGLAAALQVQLVQSGAEVKKPGESLKISCKGSGYSFTSYWI
AWVRQMPGKGLEYMGLIYPGDSDTKYSPSFQGQVTISVDKSVSTAYLQWSSLKPSDSA
VYFCARHDVGYCTDRTCAKWPEWLGVWGQGTLVTVSSGGGGSSGGGSGGGGSQSVL
TQPPSVSAAPGQKVTISCSGSSSNIGNNYVSWYQQLPGTAPKLLIYDHTNRPAGVPDRFS
GSKSGTSASLAISGFRSEDEADYYCASWDYTLSGWVFGGGTKLTVLG
```

1-98. (canceled)

99. A method of treatment to overcome resistance to trastuzumab in a patient, wherein the patient has a tumor that has acquired resistance to trastuzumab, the method comprising administering to the patient an effective amount of each of 1) an anti-ErbB3 antibody and 2) trastuzumab.

100. The method of claim 99 wherein the anti-ErbB3 antibody has a) a V_{H} CDR 1 sequence of SEQ ID NO:3, a V_{H} CDR 2 sequence of SEQ ID NO:4, a V_{H} CDR 3 sequence of SEQ ID NO:5, a V_{L} CDR4 sequence of SEQ ID NO:6, a V_{L} CDR5 sequence of SEQ ID NO:7, and a V_{L} CDR6 sequence of SEQ ID NO:8; or b) a V_{H} sequence of SEQ ID NO:1 and a V_{L} sequence of SEQ ID NO:2.

101. The method of claim 99 wherein the anti-ErbB3 antibody is selected from the group consisting of mAb 1B4C3, mAb 2D1D12, AMG-888 (U3-1287), and humanized mAb 8B8.

102. The method of claim 99 wherein an effective amount of at least one additional therapeutic agent is administered to the patient.

103. The method of claim 102 wherein the at least one additional therapeutic agent comprises a chemotherapeutic agent.

104. The method of claim 103 wherein the chemotherapeutic agent is paclitaxel.

105. A method of treatment to overcome resistance to pertuzumab in a patient, wherein the patient has a tumor that has acquired resistance to pertuzumab, the method comprising administering to the patient an effective amount of each of 1) an anti-ErbB3 antibody and 2) pertuzumab.

106. The method of claim 105 wherein the anti-ErbB3 antibody has a) a V_{H} CDR 1 sequence of SEQ ID NO:3, a V_{H} CDR 2 sequence of SEQ ID NO:4, a V_{H} CDR 3 sequence of SEQ ID NO:5, a V_{L} CDR4 sequence of SEQ ID NO:6, a V_{L} CDR5 sequence of SEQ ID NO:7, and a V_{L} CDR6 sequence of SEQ ID NO:8; or b) a V_{H} sequence of SEQ ID NO:1 and a V_{L} sequence of SEQ ID NO:2.

107. The method of claim 105 wherein the anti-ErbB3 antibody is selected from the group consisting of mAb 1B4C3, mAb 2D1D12, AMG-888 (U3-1287), and humanized mAb 8B8.

108. The method of claim 106 wherein an effective amount of at least one additional therapeutic agent is administered to the patient.

109. The method of claim 108 wherein the at least one additional therapeutic agent comprises a chemotherapeutic agent.

110. A method of overcoming resistance to trastuzumab in a patient, wherein the patient has a tumor that has acquired resistance to trastuzumab, the method comprising administering to the patient an effective amount of each of 1) a bispecific anti-ErbB3, anti-ErbB2 antibody, and 2) trastuzumab.

111. The method of claim 110 wherein the bispecific anti-ErbB3, anti-ErbB2 antibody is MM-111, which comprises the amino acid sequence of SEQ ID NO:44.

112. The method of claim 111 wherein an effective amount of at least one chemotherapeutic agent is also administered to the patient.

113. A method of overcoming resistance to an anti-EGFR antibody in a patient, wherein the patient has a tumor that has acquired resistance to the anti-EGFR antibody, the method comprising administering to the patient an effective amount of each of 1) a bispecific anti-ErbB3, anti-ErbB2 antibody, and 2) an anti-EGFR antibody.

114. The method of claim 113 wherein the bispecific anti-ErbB3, anti-ErbB2 antibody is MM-111, which comprises the amino acid sequence of SEQ ID NO:44.

115. The method of claim 114 wherein the anti-EGFR antibody of 2) is selected from the group consisting of cetuximab, MM-151, Sym004, matuzumab, panitumumab, nimotuzumab and mAb 806.

116. The method of claim 114 wherein an effective amount of a small molecule EGFR inhibitor selected from the group consisting of afatinib, lapatinib, gefitinib, canertinib, CI-1033 (PD 183805), erlotinib, PKI-166, PD-158780, pelitinib, EKB-569, and tyrphostin AG 1478 is also administered to the patient.

117. The method of claim 116 wherein the small molecule EGFR inhibitor is erlotinib, gefitinib, or lapatinib.

118. The method of claim 114 wherein an effective amount of tamoxifen is also administered to the patient.

119. The method of claim 114 wherein an effective amount of each of trastuzumab and paclitaxel are also administered to the patient.
